(19) United States
(12) Patent Application Publication
(10) Pub. No.: US 2010/0062416 A1
(43) Pub. Date: Mar. 11, 2010

(54) Immobilisation and application of antigenic carbohydrates to detect infective micro-organisms

(76) Inventors: Aldert Anthonie Bergwerff, Amersfoort (NL); Ellen van Eerden, Wageningen (NL); Peterus Johannes Nacken, Wassenaar (NL); Bertha Gerarda Maria Gortemaker, Leusden (NL); Ronaldus Bernardus Gerhardus Wolbert, Didam (NL)

Correspondence Address: TRASKBRITT, P.C., P.O. BOX 2550, SALT LAKE CITY, UT, 84110, US

(21) Appl. No.: 12/312,021

(22) PCT Filed: Oct. 24, 2007
(86) PCT No.: PCT/NL2007/050508

§ 371 Date: Jul. 29, 2009

(30) Foreign Application Priority Data
Oct. 24, 2006 (EP) 06076921.3

(51) Int. Cl. C12Q 1/70; C07K 1/04; B05D 3/00; B32B 23/04; B32B 15/02; C12M 1/34; G01N 33/566; G01N 33/53; G01N 33/48

(52) U.S. Cl. 435/5; 530/385; 427/327; 428/532; 428/403; 435/287.1; 436/501; 435/7.1; 422/82.05; 422/68.1

(57) ABSTRACT

The invention relates to the field of chemistry and diagnosis, more in particular to diagnosis of current and/or past and/or symptomless infections or of a history of exposure to a gram-negative-bacterium (such as an enterobacteriaceae or a legionella). Even more in particular, the invention relates to the screening of animals or animal products for the presence of un-wanted/undesired microorganisms. The invention further relates to a method for screening samples for the presence of antibodies directed against unwanted/undesired microorganisms and preferably such a method is performed with help of a biosensor. The invention also relates to a method for immobilising polysaccharides to solid surfaces. The invention furthermore provides solid surfaces with immobilised polysaccharides as well as applications of such surfaces.

The invention relates to the field of chemistry and diagnosis, more in particular to diagnosis of current and/or past and/or symptomless infections or of a history of exposure to a gram-negative-bacterium (such as an enterobacteriaceae or a legionella). Even more in particular, the invention relates to the screening of animals or animal products for the presence of unwanted/undesired microorganisms. The invention further relates to a method for screening samples for the presence of antibodies directed against unwanted/undesired microorganisms and preferably such a method is performed with help of a biosensor. The invention also relates to a method for immobilising polysaccharides to solid surfaces. The invention furthermore provides solid surfaces with immobilised polysaccharides as well as applications of such surfaces.

The world is full of gram-negative bacteria, many of which are members of the family Enterobacteriaceae. Members of this family are found in the gastrointestinal tract of animals, but many are also free living in soil and water. Members of the family Enterobacteriaceae have very complex antigenic structures. Moreover, they comprise multiple antigens that are identified as K antigens, H antigens and O antigens. The K antigen is the acidic polysaccharide capsule. The capsule has many functions including evasion from the immune system of the infected host and adhesion to the epithelium of the host. The H antigen is located on the flagella.

The outer portion of the cell wall in gram-negative bacteria is chiefly composed of lipopolysaccharides (LPS). LPS is composed of lipid A which is buried in the outer membrane, a short carbohydrate core and optionally a chain of polysaccharides that is made up of repeating units. The O-antigens are located on the polysaccharide. Lipid A is the toxic constituent of the LPS. As cells lyse, LPS is released, leading to fever and complement consumption. It also interferes with coagulation and at high concentrations eventually leads to a state of shock.

As a non-limiting example one member of the enterobacteriaceae, salmonella, will be discussed in more detail. A large number of the subspecies of the genera of Salmonella enterica are important pathogenenic bacteria for humans and animals. Besides that animals go into a pathological episode, animals can be symptomless carriers of the bacteria. Contaminated animals can be a source of these pathogens threatening public health for example through the food that these animals produce. As many stakeholders consider the number of food-borne salmonella infections unacceptable, measures have to be taken to contain this pathogen in the food chain.

Salmonella is of major significance as a pathogenic microorganism in food-borne infections in humans, causing mild to severe clinical effects. In The Netherlands, 5% of all identified cases of gastroenteritis is salmonellosis (Edel et al., 1993; Hoogenboom Verdegaal et al., 1994). The average incidence of this infection is 450 cases per 100,000 person years at risk, which is similar to that in other industrialized countries (Berends et al., 1998). Despite the 2480 serotypes identified in the group of S. enterica up to 2001 (Popoff, 2001), only a small number have been involved in human infections (Grimont et al., 2000). Salmonella typhimurium plus Salmonella enteritidis represented >75% of all salmonella isolates from human sources sent to the Dutch National Salmonella Centre at the RIVM in 2002 (Van Pelt et al., 2003). This percentage consisted of 51% contributed by contact with chicken products (poultry 15%; eggs 36%)(Van Pelt et al., 2003).

Detection of immunoglobulins in the body fluids of organisms (serology) is a way to establish a history of exposure of animals and humans to infectious agents. A humoral response against salmonella antigens can be detected in chickens 1 week post-infection and persists for at least 10 weeks even if the bird is no longer culture-positive (Holt, 2000). The antigenic determinants of salmonella are, as described above, composed of somatic (O), flagellar (H) and surface (Vi) antigens (Holt, 2000). Variations in the composition of antigens correlate with different salmonella serotypes.

Typically, serology is faster than culture-typing of the disease-causative organism. Fast and specific detection of potential salmonella-positive herds and flocks is of importance in order to take adequate measures in production processes. The detection of antibodies in serum and blood samples from food-producing animals reporting the presence of zoonotic pathogens is therefore of significance. Such information is then used as the input for risk-assessment and rational slaughtering of potentially pathogen-contaminated animals in order to be able to increase food safety, but also to improve occupational hazards and to reduce spreading of the pathogens in the environment.

A number of serological tests have been developed for the detection of invasive salmonella species. Among many such methods, agglutination and ELISA have most commonly been used (Barrow, 2000). Agglutination tests have been used successfully to eradicate Salmonella pullorum from poultry flocks. However, the approach is cumbersome, laborious and not suitable for large-scale screening programs according to modern standards. Several ELISA procedures, which are considered relatively cheap and fast, have therefore been developed to detect anti-S. enteritidis and S. typhimurium antigen responses in poultry sera (Barrow et al., 1996; Thorns et al., 1996; de Vries et al., 1998; Barrow, 2000; Yamane et al., 2000).

The use of biosensors also promises to be useful, cheap and rapid in this area of analysis. In addition, the technique is able to detect multiple analytes of any biomolecular type in a single run. A biosensor is defined as an analytical device consisting of (i) a re-usable immobilized biological ligand that ‘senses’ the analyte, and (ii) a physical transducer, which translates this phenomenon into an electronic signal.

The surface plasmon resonance (SPR) phenomenon was first recognized in the early 1960s (Kretschmann and Raether, 1968) and the first SPR biosensors were introduced in the 1980s (Liedberg et al., 1983). It took until the late 1980s and early 1990s before the first commercially available SPR-based biosensor equipment was released on the market. Initially, this type of biosensor attracted the interest of pharmaceutical companies as a secondary tool for both selective and sensitive in vitro screening of promising novel pharmaceutical products from combinatorial libraries. It proved to be a valuable alternative for classic approaches such as ELISA procedures. Moreover, it offers real-time measurement of the binding event in contrast to end-point determinations. The benefits of this analytical approach have also been recognized by many other life science disciplines, including food sciences (Ivnitski et al., 1999; Medina, 1997). So far, only a few publications on SPR biosensing have addressed the detection of pathogenic microorganisms, for example the use of immobilized Escherichia coli O157:H7 cells to screen the performance of anti-E. coli O157:H7 antibodies (Medina et al., 1997), and the use of these antibodies to detect E. coli O157:117 cells (Fratamico et al., 1997). In Jongerius-Gortemaker et al. (2002) a study to the suitability of an SPR optical biosensor to detect antibodies in serum and blood indicating a humoral reaction to invasion with Salmonella serotypes enteritidis and typhimurium was initiated. In this study, use was made of immobilised flagellar antigen fusion proteins. After thorough analysis it was concluded that the sensitivity and/or the robustness of this system was not sufficient and in particular not for high-throughput screening of for example poultry at the slaughter line in an abattoir, processing animals at the rate of several thousands per hour.

The goal of the present invention is to provide for a method that has an improved sensitivity and/or an improved robustness. This goal has been reached by developing a carrier with immobilised somatic or so-called O-antigens. As described, the O-antigens are located on the lipopolysaccharides and the composition of the polysaccharide varies and corresponds with the serovar of the salmonella (sub)species. Every serotype can, amongst others, be described by a number of O-antigens and are typically coded with a number, such as O4, O6 or O12. The O-antigens can be found as repeating units on the polysaccharide part of the LPS. The length of the polysaccharide also varies and can be between zero (rough LPS) and more than 50 repeating units (smooth LPS).

Within the Salmonella enterica family, different serogroups can be distinguished; each group comprises at least one specific O-antigen. The salmonella serovars of importance in chicken and pigs are listed with their O-antigen profile in Table 1. In Denmark, Germany, Greece and The Netherlands, 39.5% of all salmonella-positive pigs sampled at the abattoir were determined as S. typhimurium. Dependent of country, other important isolates from pigs were S. derby (17.1%), S. infantis (8.0%), S. panama (5.1%), S. ohio (4.9%), S. London (4.4%), S. livingstone (3.1%), S. virchow (2.7%), S. bredeny (2.1%), S. mbandaka (1.1%), S. Brandenburg (1.0%), S. goldcoast (0.8%).

In case of chickens, 14% of the chickens were salmonella-positive at flock level in 2002 in The Netherlands. The predominant serovar was in that case S. paratyphi B var. java. At the retail level a comparable percentage (13.4%) was found in the Netherlands. The most frequent salmonella serovars isolated from broilers in 14 EU member states were S. paratyphi B var. java (24.7%), S. enteritidis (13.6%), S. infantis (8.0%), S. virchow (6.7%), S. livingstone (5.7%), S. mbandaka (5.5%), S. typhimurium (5.3%), S. senftenberg (5.0%), S. hadar (3.7%). S. paratyphi B var. java is dominating, but this is fully attributable to The Netherlands.

TABLE 1
Some salmonella serovars considered as important zoonotic agents in
broilers and in pigs listed with their O-antigen profiles (Popoff, 2001)

| Salmonella | Chicken (C)/pigs | O-antigen | |
|---|---|---|---|
| serovar | (P) | profile | serogroup |
| Brandenburg | P | 4, [5], 12 | B |
| Bredeny | P | 1, 4, 12, 27 | B |
| Derby | P | 1^{a}, 4, [5]^{b}, 12 | B |
| Enteritidis | C | 1, 9, 12 | D₁ |
| Goldcoast | C/P | 6, 8 | C₂ |
| Infantis | C/P | 6, 7, 14 | C₁ |
| Livingstone | P | 6, 7, 14 | C₁ |
| London | P | 3, 10, [15] | E₁ |
| Mbandaka | P | 6, 7, 14 | C₁ |
| Meleagridis | P | 3, 10, [15], [15, | E₁ |
| | | 34]^{c} | |
| Ohio | P | 6, 7, 14 | C₁ |
| Panama | P | 1, 9, 12 | D₁ |
| Paratyphi B var. | C | 1, 4, [5], 12 | B |
| Java | | | |
| Typhimurium | C/P | 1, 4, [5], 12 | B |
| Virchow | P | 6, 7, 14 | C₁ |

^{a}O antigen determined by phage conversion is indicated by underlining
^{b}O antigens which may be present or absent are indicated in square brackets
^{c}lysogenized by phage ε15 [15] and by phage ε34 [15, 34]

In a first embodiment, the invention provides a method for immobilisation of a polysaccharide on a carrier, comprising contacting said polysaccharide with an oxidising agent and a polymer comprising at least two amine and/or amide groups to obtain a polysaccharide-polymer complex and coupling said polysaccharide-polymer complex to said carrier. The polymer can be any polymer that contains at least two amine and/or amide groups. Said at least two amine and/or amide groups preferably cross-link said polymer to said polysaccharide and said carrier. To allow for more efficient coupling it is preferred that said polymer comprises at least 4 and more preferably at least 7 amine or amide groups. The polymer comprises at least 10 building blocks. Building blocks of a polymer share characteristic reactive groups that enable elongation of the polymer. A preferred building block is an amino acid or a functional part, derivative and/or analogue thereof. In a preferred embodiment said polymer comprises a protein. A protein comprises at least one polypeptide chain comprising at least 10 amino acids or functional equivalent thereof. A protein contains at least constituents having free amine and/or amide groups, such as e.g. Asn (A), Lys (K), Arg (R), Gln (Q). In the context of the invention the protein can also be a multimer comprising at least two polypeptide chains that are covalently or non-covalently linked to each other. The protein may comprise modifications such as those common to biological systems such as post-translational glycosylation. The protein may also be artificially modified or provided with a further group as long as it has the mentioned amine and/or amide groups available.

In a preferred embodiment said polysaccharide is derived from a gram-negative bacterium. The sensitivity of such a prepared carrier is much improved when the lipopolysaccharide (O-antigen) before the immobilisation on the carrier is oxidised in the presence of a polymer comprising at least two amine and/or amide groups, preferably a protein. Although we do not wish to be bound by any theory, it is currently thought that the aldehyde groups that result from the oxidation of the polysaccharides are capable of reacting with the amino groups of the protein to form a substituted imine (Schiff-base binding). Upon injection over (an activated) carrier (for example a sensorchip) the available aldehyde groups react with hydrazide to form hydrazon. The following reduction stabilises not only the covalent binding between the carrier (for example a carrier comprising dextran) and the polysaccharide but also the imine binding between protein and polysaccharide. As will be explained in more detail in the experimental part, polysaccharides (O antigens) of different salmonella sera types have been immobilised on a carrier. The prepared carriers were subsequently subjected to an SPR-analysis with standard sera. The obtained serological response was used as an indicator for success of the method. When coupling reactions were performed without the oxidation step no or almost no significant response of reference sera could be detected.

Preferably, the immobilisation/coupling of the polysaccharide-protein complex to a carrier is such that high sensitivity and/or robustness is obtained. Whereas flagellar antigens denature and lose their antigenicity towards serum antibodies while the sensor chip has to be regenerated for a next analysis cycle with relatively harsh solvents, the somatic antigens are found rather stable towards these regeneration solvents. In fact, the loss of immobilized O-antigen activity is believed to be primarily associated with degradation of the solid surface, namely gradual loss of dextran layer attached to the goldfilm, to which the antigens are bound. The method according to the invention results in a carrier that is more robust compared to a carrier of the prior art.

Preferably, the invention provides a method for immobilisation of a polysaccharide on a carrier, comprising contacting said polysaccharide with an oxidising agent and a protein to obtain a polysaccharide-protein complex and coupling said polysaccharide-protein complex to said carrier, wherein said polysaccharide is derived from a gram-negative bacterium and even more preferably wherein said polysaccharide is derived from an enterobacteriaceae. Yet even more preferably, said polysaccharide is derived from a gram-negative bacterium that is a human or veterinary or plant pathogen. Examples of such polysaccharides are polysaccharides derived from a salmonella (sub)species.

Other examples are polysaccharides derived from Eschericia coli species (for example E. coli O157) and the bacterial species outlined in Table 2.

TABLE 2
Examples of LPS-containing bacteria pathogenic to human and/or
animals.

| Bacterial species | Mainly found in | Affecting |
|---|---|---|
| Campylobacter coli | Swine | Humans |
| Campylobacter jejuni | Avian species, dogs | Humans |
| Campylobacter lari | Seagull | Humans |
| Escherichia coli O157 | Ruminants | Humans |
| Legionella pneumophila | Water | Humans |
| Salmonella choleraesuis | Swine | Swine |
| Salmonella enteritidis | Avian species, swine | Humans |
| Salmonella gallinarum | Avian species | Chickens |
| Salmonella goldcoast | Swine | Humans |
| Salmonella infantis | Chickens | Humans |
| Salmonella livingstone | Swine | Humans |
| Salmonella meleagridis | Swine | Humans |
| Salmonella pollorum | Avian species | Chickens |
| Salmonella typhimurium | Avian | Humans, horses |
| Streptococcus suis | Swine | Swine, humans |
| Vibrio cholerae (non-O1) | Aquatic animals | Humans |
| Vibrio paraheamolyticus | Aquatic animals | Humans |
| Vibrio vulnificus | Aquatic animals | Humans |
| Yersinia enterocolitica | Swine | Humans |

As will be explained in more detail later, a carrier comprising an immobilised polysaccharide (O-antigen) is particularly useful in the diagnosis of the mentioned LPS-containing bacteria.

The term “polysaccharide” is intended to mean an entity comprising two or more glycoside linked monosaccharide units and embraces, amongst others, an oligosaccharide (2-10 residues) as well as a polysaccharide (more than 10 monosaccharides). The linking may result in linear or branched polysaccharide. In a preferred embodiment, the invention provides a method for immobilisation of a polysaccharide on a carrier, comprising contacting said polysaccharide with an oxidising agent and a protein to obtain a polysaccharide-protein complex and coupling said polysaccharide-protein complex to said carrier, wherein said polysaccharide is a lipopolysaccharide (LPS), i.e. a polysaccharide comprising lipid A. It is clear to a skilled person that the used (lipo)polysaccharide must comprise at least one antigenic structure and one group available/suitable for providing a linkage between the protein and the polysaccharide. More details in respect of the last item will be provided later on. Hence, as long as the (lipo)polysaccharide comprises an antigenic structure and a group suitable for providing a linkage between the protein and the polysaccharide an immobilization method of the invention may be used to obtained a sensitive and/or robust carrier.

The LPS is expressed at the cellular exterior and is part of the bacterial cellular wall. The expression of LPS is not under direct genetic control, so that LPS is a pool of different molecules with varying composition of the lipid A part in terms of the attached aliphatic chain. Moreover, the bacterial cell may synthesize rough LPS with no or a short carbohydrate chain, or smooth LPS with a mature carbohydrate chain existing of more than 50 repeating units expressing its antigenicity. In addition to this heterogeneity, within a single molecule LPS, several O-antigen entities, which are distinctively numbered, may be expressed. An O-antigen profile is, however, per definition unique for a salmonella serogroup. A complete serotyping of a salmonella also includes the H-antigens as well as the Vi-antigens.

LPS may be obtained by a variety of methods and the experimental part describes in more detail the use of a trichloric acid extraction (optionally followed by ethanol extraction and dialysis) according to Staub (1965) for this purpose. Other examples of suitable extraction methods are described by Wilkons (1996) and include, but are not restricted to, extractions with diethylene glycol, dimethyl sulphoxide, NaCl-diethyl ether (1:2 (v/v)), NaCl-butan-1-ol (1:1 (v/v)), aqueous EDTA, NaCl-sodium citrate, aqueous phenol or aqueous phenol-chloroform petroleum.

The purity of the obtained/used LPS batch is considered not to be extremely critical. It is experienced that the LPS does not have to be completely free of contaminants. The specific coupling reaction provides a certain degree of selectivity. Moreover, as described in the experimental part, the used/obtained LPS (preferably an LPS batch) is optimised in respect of the amount of Protein necessary for an optimal response. It is clear to a skilled person that the LPS preferably comprises not much rough LPS. The preferred LPS batch essentially comprises smooth LPS.

Although we do not wish to be bound by any theory it is currently thought that the presence of a 2-keto-3-deoxy-octonic acid (KDO) and/or a glycerol-mannoheptose (Hep) and/or a GlcNAc in the core of the LPS molecule is needed for a covalent coupling.

Although a lot of different bacteria are employed by the term gram-negative bacteria it is believed that LPS from all these bacteria are suitable for use in the presently claimed invention as long as the LPS comprises at least one constituent with non-conjugated or de-conjugated vicinal hydroxy groups, preferably in the core region of the LPS molecule. In salmonella, most likely candidate constituents are KDO and Hep and GlcNAc residues. The presence or absence of such a KDO and/or Hep and/or GlcNAc group is indirectly genetically determined. Although the genetic information necessary to construct the species-, serotype or even strain specific monosaccharides is present in the corresponding organism, it depends on the growth circumstances whether said LPS contains aldehyde-convertible monosaccharides in the core region.

There are of course also other sources of LPS available, such as buying it commercially.

In a preferred embodiment, the invention provides a method for immobilisation of a polysaccharide on a carrier, comprising contacting said polysaccharide with an oxidising agent and a protein to obtain a polysaccharide-protein complex and coupling said polysaccharide-protein complex to said carrier, wherein said protein is a protein (for example a serum protein) with a certain amount of (primary) amines. Preferably, at least some of these amines are not sterical hindered and/or are not participating in non-covalent bindings, such as H—H bridges or dipole-dipole interactions and/or are not protonated to amine cations. Such a protein preferably does not have or hardly have, any immunogenic properties and hence cross-reacting antibodies directed to the used protein are avoided as much as possible. Examples of suitable proteins are haemoglobin (Hb), ovalbumin (Ob), myoglobin (Mb) and serum albumin (SA). The biosensor response of different standard sera on immobilised LPS oxidized in the presence of Hb or Ob or Mb or SA were determined. Serum albumin, myoglobin and haemoglobin gave the most promising results. In a preferred embodiment the protein is haemoglobin or myoglobin.

The necessary protein is obtained commercially or by (overexpressing) in a suitable expression system or by isolating it from a suitable source. Haemoglobin has for example been obtained by isolating it from blood. Preferably the used protein batches are as pure as possible, thereby circumventing as much cross-reactions as possible. It is however experienced that small amounts of contamination are allowed without jeopardising the sensitivity and/or robustness of the obtained carriers.

The ratio (lipo)polysachcharide versus protein depends, amongst others, on the used protein. Experiments with Hb have shown that concentrations between 15 and 50% (m/m) have resulted in satisfactory results. When bovine serum albumin is used much lower ratios, between 0.7 and 7% (m/m), are used. Some examples: the optimal Hb concentration for S. livingstone LPS is around 50% (m/m) and for S. enteritidis LPS the optimal Hb concentration is 15% (m/m).

The isolated LPS preparations are preferably oxidised in the presence of a protein facilitated by an oxidising agent. In a preferred embodiment the invention provides a method for immobilisation of a polysaccharide on a carrier, comprising contacting said polysaccharide with an oxidising agent and a protein to obtain a polysaccharide-protein complex and coupling said polysaccharide-protein complex to said carrier, wherein said oxidising agent is capable of oxidising vicinal diols. Even more preferably, the oxidising agent preferably oxidises vicinal diols at least under controlled condition. Oxidation of vicinal diols is preferred as this warrants reliable coupling of vicinal diol containing polysaccharide to the matrix. In a preferred embodiment of the invention the polysaccharides to be coupled to the matrix contain an antigen that is to be recognised by a member of a binding pair. To be recognizable it is preferred that the antigen is left unchanged at least in the majority of the polysaccharides that are being coupled to the carrier. This requires a balance between the level of oxidation required to obtain efficient coupling to the matrix and availability of the antigen for association with the member of the binding pair. The latter requires that the antigen is left essentially unaffected by the oxidation at least in an amount sufficient to be usable in a diagnostic setting. Oxidation of vicinal diols according to the present invention warrants the availability of sufficient antigen in recognizable form while at the same time allowing efficient coupling of the polysaccharide to the carrier. In a preferred embodiment said oxidising agent comprises (sodium) m-periodate. Other periodates such as potassium periodate or other salts thereof are also suitable periodates of the present invention. Periodate oxidation is very suited for enabling preferential oxidation of vicinal diols according to the present invention. Oxidation of predominantly vicinal diols in a polysaccharide of the invention can typically be achieved by incubating said polyssaccharide with said periodate at a concentration of between 1 and 10 mM periodate. Other parameters of the reaction influence both the speed and the type of reaction predominantly performed. One example is incubation time. When applying very short incubation times higher than 10 mM periodate can be used. Periodate preferably oxidises vicinal diols, particularly of the more susceptible vicinal diols in the side chains of the polyssacharide. Thus as long as so-called ‘mild’ reaction conditions are chosen, preferably vicinal diols will be oxidised. When conditions are chosen that also allow other oxidation reactions to occur more often (for instance because of depletion of the vicinal diol substrate), the antigen present in the polysaccharide will be affected significantly. Thus for the present invention a periodate oxidation is said to be mild when the mentioned preferred concentrations are used and when at least 20% and preferably at least 50%, more preferably at least 70% and most preferably about 90% of the antigen is intact after oxidation. Availability or intactness of the antigen is preferably measured by means of an ELISA assay using a standardized antibody. Again we do not wish to be bound by any theory but it is currently thought that periodate will induce an oxidative disruption of linkages between vicinal diols on especially carbohydrate moieties, as in e.g. mannose, to yield aldehyde functionalities. This reaction is typically performed in buffers at a pH range between 4.5 and 5.5 in the dark using a (preferably) freshly prepared 1-100 mM sodium meta-periodate in 0.1 M sodium acetate. Preferably the reaction, is performed at a concentration of between 1 and 10 mM metaperiodate. The oxidation is performed in the presence of a protein in the ranges as discussed above. The bis-aldehyde compounds, like the oxidised monosaccharide constituents in the polysaccharide chain of LPS, may react with any amino group in a protein and may form a Schiff-base linkage resulting in a substituted imine. When one or both of the vicinal hydroxyl groups is condensed in a covalent sugar linkage, the hydroxyl function is lost and no oxidation occurs. This is the case in many branched and/or linearly linked oligo- and polysaccharides. In the case of salmonella LPS, the inner core structure carries in most cases an oxidisable Gal, GlcNAc, Hep and/or KDO, but non-reducing Hep and KDO constituents are most susceptible for oxidation, in particular at very mild oxidation conditions at concentrations less than 6 mM meta periodate. Because the core region is a rather conserved part of LPS from different Enterobactericeae, (lipo)polysaccharides of members of the Enterobactericeae may be applied in a method of the invention.

Periodate will also oxidise, when present, certain aminoethanol derivatives such as the hydroxylysine residues in collagen, as well as methionine (to its sulfoxide) and certain thiols (usually to disulfides). In addition, N-terminal serine and threonine residues of peptides and proteins can be selectively oxidized by periodate to aldehyde groups. These reactions, however, usually occur at a slower rate than oxidation of vicinal diols and the presence of such group does not substantially interfere with a method according to the invention.

The invention also provides a method for immobilisation of a polysaccharide on a carrier, comprising contacting said polysaccharide with an oxidising agent and a protein to obtain a polysaccharide-protein complex and coupling said polysaccharide-protein complex to said carrier, further comprising a step which results in ending/stopping the oxidation process, for example by desalting of said polysaccharide-protein complex. This is for example accomplished with help of a NAP-5 column. However the person skilled in the art is aware that many other methods exist which have the same effect, for example adding a reductor or an easily oxidisable molecule such as glycerol. Preferably, the way of stopping the oxidation is such that at the same time a buffer change is accomplished, for example HPLC, FPLC, dialysis, ion-exchangers, gel electrophoresis or ultrafiltration.

For storage purposes, the production of evaporated aliquots, after addition of protein, is also described within the experimental part. This results in the presence of a large stock of reproducible material.

The invention therefore further comprises the obtained intermediate, i.e. the preparation of in the presence of protein oxidised polysaccharide, optionally desalted and optionally evaporated.

Preferably, the used carrier is made of an inert, non-hydrophobic material and the binding of the LPS-protein complex to said carrier is covalent. Even more preferred such a carrier has a low protein binding or low biomolecular binding. Examples are a carrier of glass or silica or of a non-hydrophobic plastic. In a preferred embodiment said carrier is in the form of a microsphere or bead. Several types of microsphere or beads are available to the person skilled in the art. In a preferred embodiment said microsphere or bead comprises polystyrene. Microsphere or beads are particularly preferred because they can be provided with different antigens using a method of the invention. Microsphere or beads with different antigens can be accordingly coded with a different colour (for example a(n) (internal) fluorescence label). The presence of differently coloured labeled beads or microspheres facilitates the identification of the different beads or microspheres. Preferably, the differently coloured beads or microspheres have been provided with different antigen via a method of the invention. However, beads or microspheres that have been provided with different antigens can also be identified by using beads or microspheres with different sizes. In one of the preferred embodiments, identification of beads or microspheres is accomplished via a combination of size and (internal) fluoresecence labels. Preferably (advanced) flow cytometry is used. Testing a sample for the presence of an antibody against an antigen can be done using a collection of the mentioned microsphere or beads. Binding of the antibody to a particular type of antigen can now be detected easily by the colour code of the microsphere or bead bound. Binding of the antibody can be detected in various ways. For instance, microsphere or beads containing bound antibody can be extracted from the sample and measured using a further antibody specific for the constant region of the antibody. On the other hand, sample can be directly analysed, i.e. in the absence of further manipulations by labelling the bound antibody and simultaneously detecting colour of the antibody and the colour of the microsphere or bead. Various methods for simultaneous detection of two or more colours are available to the person skilled in the art. In the present invention, a colour is defined as any type of electromagnetic radiation that can be detected, be it a typical colour revealed, for instance, by reflection of light, to light emitted as a result of fluorescence or phosphorescence.

The invention thus further provides a collection of at least two microsphere or beads wherein at least two of said at least two microsphere or beads each comprise a different antigen of the present invention. In a preferred embodiment said antigen comprises O-antigen of Salmonella. In a particularly preferred embodiment said antigen is linked to said microsphere or beads carrier using a method of the invention. Thus preferably at least one of said microsphere or beads comprises a polysaccharide coating linked to a polysaccharide comprising an antigen to be detected linked to each other via a polymer comprising at least two amine and/or amide groups, preferably a protein of the invention, wherein said linkage polymer (protein) is linked to said polysaccharide comprising said antigen, via an amine and/or amide group on said polymer and a periodate oxidised vicinal diol on said polysaccharide comprising said antigen.

As described above, the present invention provides means to detect multiple, different antibodies at the same time. However, it is also possible to use one type of carrier that has been provided with one type of antigen. i.e. also one type of microsphere or one type of bead (obtainable by a method of the invention) is useful, for example in diagnosis of one particular serovar.

In a preferred embodiment the invention provides a method for immobilisation of a polysaccharide on a carrier, comprising contacting said polysaccharide with an oxidising agent and a protein to obtain a polysaccharide-protein complex and coupling said polysaccharide-protein complex to said carrier, further comprising activating the surface of said carrier. In an even more preferred embodiment, said carrier comprises a glass surface coated with gold and even more preferred said carrier is modified with a carboxyl donor. A surface can be activated. Carboxylic acid (COOH) groups (further referred to as carboxyl groups) are needed on this surface. Preferably these COOH groups are provided by a stable homogeneous layer of molecules, which may have been modified for this purpose. These surfaces may exist of, but are not limited to, carboxylic acid-modified polysaccharides, alkanes or alkenes, such as polyethylene, attached to e.g. gold, polystyrene or silicon surfaces. Preferably the carrier comprises a polysaccharide that acts as a carboxyl donor. More preferably a carboxymethylated dextran layer wherein said polysoaccharide modified carrier, preferably comprising a dextran layer is activated with 1-ethyl-3-(3-dimethylaminopropyl)carbodiimide hydrochloride and N-hydroxysuccinimide.

The activation is preferably followed by preparation with carbohydrazine. In the next step the polysaccharide-protein complex is added to the activated dextran layer. The reactive aldehyde functionalities react spontaneously with the hydrazide to hydrazones, which are then reduced to stabilise the covalent bonds.

Prior to routinely use, the performance of chip-conjugated LPS to bind anti-Enterobacterium (for example salmonella) antibodies is assessed using reference polyclonal agglutination sera.

As will be explained in more detail in the experimental part herein, binding of the obtained (lipo)polysaccharide-protein complex to a carrier can be performed via COOH as well as via NH2 groups or via the combination of COOH and NH2 groups. In a preferred embodiment, a carrier used in a method of the invention comprises COOH and/or NH2 groups that are functionally available for binding. In yet another preferred embodiment, a COOH carrier (for example COOH beads) is used for the testing or screening of chicken sera. In a further preferred embodiment, a NH2 carrier (for example NH2 beads) is used in the analysis of procine sera.

Depending on the analytical/diagonostic question asked it is decided whether one or for example at least two different serogroup-representing carbohydrates, preferably 4 different serogroup-representing carbohydrates are used. In case one is interested in knowing which particular serogroup is present, multiple (the amount of which is different on the particular question asked and on the used apparatus) different serogroup-representing carbohydrates are used and if one just wants to know whether for example an animal is or has been infected by a particular serogroup, a single serogroup-representing carbohydrate may be oxidised in the presence of a protein and immobilised on a carrier. The use of at least two different serogroup-representing carbohydrates results in a carrier that can be used in a multi-serogroup analysis. More preferably at least three and even more preferred at least more than three (for example four or five) different polysaccharides are used. These polysaccharides may be oxidised in the presence of one type of protein or in the presence of different types of protein. The skilled person is capable of making any sensible combination. For example, to be able to detect more than 90% of all salmonella infections serogroups B, C and D in chicken and serogroups B, C, D and E in pigs should be represented.

Using one type of serogroup-representing carbohydrates is extremely useful if one is interested in the question whether or not a certain type of bacterium is or was present. Using multiple different serogroup-representing carbohydrates is for example useful if one wants to determine whether an animal is or was infected by any gram-negative bacteria (for example enterobacteriaceae).

In a preferred embodiment the invention provides a method for immobilisation of a polysaccharide on a carrier, comprising contacting said polysaccharide with an oxidising agent and a protein to obtain a polysaccharide-protein complex and coupling said polysaccharide-protein complex to said carrier, wherein said carrier is a biosensor chip. Such a biosensor chip is commercially available (for example that produced by Biacore) and hence no further information will be provided.

In another embodiment the invention provides a carrier obtained by the method according as described above or a carrier comprising an immobilised polysaccharide-protein complex on its surface. In one embodiment of the invention a carrier of the invention comprises a polysaccharide coating that is linked to a further polysaccharide coating via reductive amination, wherein said further polysaccharide coating comprises a protein coupled to said further polysaccharide coating through oxidation of vicinal diols on said further polysaccharide-protein complex. In a preferred embodiment said reductive amination is achieved. In a preferred embodiment the invention provides a carrier comprising a polysacharide coating that is coupled to polysaccharide.

In yet another embodiment the invention provides biosensor comprising a carrier according to the invention. Whether the carrier is obtained by a method according to the invention can for example be determined by extracting the polysaccharides from said carrier and determining whether covalently linked protein is present. As already discussed above the carrier may also comprise different immobilised polysaccharides (for example O-antigens) possibly in combinations with different types of protein. However, also one type of protein may be used in the oxidation of different polysaccharides.

Whether a carrier and/or chip of the invention is employed can for example be determined with help of MALDI-MS possibly in combination with proteolytic digestion. Such an analysis provides information with respect to the used protein and polysaccharide. With help of acidic hydrolysis the polysaccharide-protein complexes are released from the carrier. Such an obtained mixture is then subjected to LC-MS/MS analysis before and after proteolytic hydrolysis. The obtained complex may also be subjected to a monosaccharide analysis, for example GC-MS following methanolysis and/or Smith degradation, from which it is determined which type of LPS is used. This information is furthermore used to determine whether KDO, Hep or other sugars have been oxidised.

A carrier of the invention may be used in different detection systems, for example optical, thermal, acoustic, amperometric, magnetic or chemical and a carrier of the invention may be used in any biomolecular interaction assay (BIA) or any affinity assay (AA). As a non-limiting example, the use of optical detection via Surface Plasmon Resonance is described in more detail.

The invention provides a Surface Plasmon Resonance detection system comprising a biosensor as described above. The gold layer in the sensor chip creates the physical conditions required for Surface Plasmon Resonance (SPR). The principle of SPR will be described in the context of Biacore instruments. They incorporate the SPR phenomenon to monitor biomolecular interactions in ‘real-time’. At an interface between two transparent media of different refractive index such as glass and water, light coming from the side of higher refractive index is partly reflected and partly refracted. Above a certain critical angle of incidence no light is refracted across the interface and total internal reflection (TIR) occurs at the metal film-liquid interface. This is where light travels through an optically dense medium such as glass, and is reflected back through that medium at the interface with a less optically dense medium such as buffer. Although the incident light is totally reflected, the electromagnetic field component, termed the evanescent wave, penetrates a distance on the order of one wavelength into the less optically dense medium. The evanescent wave is generated at the interface between a glass prism (high refractive index) and a layer of buffer (lower refractive index). If the interface between the media of higher and lower refractive indices is coated with a thin metal film (a fraction of the light wavelength), then the propagation of the evanescent wave will interact with the electrons on the metal layer. Metals contain electron clouds at their surface, which can couple with incident light at certain angles. These electrons are also known as plasmons, and the passage of the evanescent wave through the metal layer causes the plasmons to resonate, forming a quantum mechanical wave known as a surface plasmon. Therefore, when surface plasmon resonance occurs, energy from the incident light is lost to the metal film resulting in a decrease in the reflected light intensity. The resonance phenomenon only occurs at an acutely defined angle of the incident light. This angle is dependent on the refractive index of the medium close to the metal-film surface. Changes in the refractive index of the buffer, solution (e.g. an increase in surface concentration of solutes), to a distance of about 300 nm from the metal film surface will therefore alter the resonance angle. Continuons monitoring of this resonance angle allows the quantitation of changes in refractive index of the buffer solution close to the metal-film surface. In ‘real-time’ Biacore, the metal film properties, wavelength, and refractive index of the glass (denser medium) are all kept constant, and as a result SPR can be used to monitor the refractive index of the aqueous layer immediately adjacent to the metal (gold) layer. In the Biacore system the chip is composed of glass, has 4 channels and the associated gold layer is covered with a layer of dextran chemically modified to facilitate immobilisation of ligands such as antibodies or antigens. Any changes in mass that occur due to binding of the analyte with the immobilised antibody on the sensor chip will cause a change in SPR angle, which is monitored in ‘real-time’ and quantified as a sensorgram. A mass change of approximately 1 kRU (1,000 RU) corresponds to a mass change in surface protein concentration of 1 ng/mm². Typical responses for surface binding of proteins are of the order of 0.1-20 kRU.

There is no need to label molecules with fluorescent or radioactive tags—so avoiding the possibility that labels may compromise activity and moreover no difficult or expensive chemistry is necessary for labelling.

Besides the above mentioned biosensor two other, non-limiting examples of a suitable carrier are provided.

One example of a suitable carrier is an ImmuSpeed™ chip which is commercially available (for example produced by DiagnoSwiss). Such a chip comprises parallel channels etched into a polymer substrate (a method for obtaining these kinds of chips is provided in EP 1 255 690 B1). If necessary, the surface is first treated to introduce —COOH groups at its surface. One of the used polymers is polyimide which can be treated such that —COOH groups are introduced. One non-limiting example for the introduction of —COOH groups on a polyimide surface is by treating said surface with an aqueous solution containing sodium hydroxide NaOH (hydrolysis). After this alkaline hydrolysis, NaOH is removed and carboxylic groups protonated by, for example, flowing 0.25 M acetic acid for 5 min through the channels of the chip at a flow rate of 10 μL/min using cycles each consisting of 2 s pumping and 15 s arrest. The chip surface is then rinsed with PBS at 10 μl/min through 2-s flow and 10-s stop cycles for 5 min. When the —COOH groups have been introduced, the polysaccharide-polymer complex is (covelently) bound as described earlier. In short, the carboxyl group containing polymeric surface is modified with 1-ethyl-3-(3-dimethylaminopropyl)carbodiimide hydrochloride and N-hydroxysuccinimide. This activation is followed by passing a solution containing carbohydrazine over the surface. In the next step, the polysaccharide-protein complex is added to the activated polymeric surface. The reactive aldehyde functionalities reacts with the hydrazide to hydrazones, which is then reduced to stabilise the covalent bonds.

By using a covelent binding of the polysaccharide-polymer complex to the carrier, the obtained chip can be used multiple times (preferably more than 1,000 times) by regenerating the prepared chip after it has been used. Normally, the immuchips are for single use only (i.e. they are disposed after use). The present invention thus shows an improved use of the Immuchip by decreasing the amount of waste.

The above given description results in a covalent binding of the polysaccharide-polymer complex to the polyimide comprising carrier. Although a covalent binding is preferred for surface regeneration and automation purposes, a stable coating of the acquired protein-LPS complex is also obtained through the presence of the said protein in this complex. Although not a limiting series, solid carriers comprising standard organic materials such as polyethylene terephthalate (PET), polycarbonate, polyethylene (PE), polystyrene, cellulose acetate or polyimide can be exploited for the coating of the protein-LPS complex.

We have performed experiments with a non-covalent bound polysaccharide-polymer complex to a polyimide surface and the obtained carrier could be regenerated at least 4 times.

One of the advantages of the ImmuSpeed™ versus the earlier described Biacore biosensor, is that the amount of serogroups that can be tested at the same time can be increased to for example 5 (for example B, C1, C2, D and E).

The detection of an ImmuSpeed™ chip is based on amperometrics.

As already described, one way for determining the amount of bound antibody to a carrier of the invention is by using a second antibody which has been labelled. Alkaline phosphatase is a suitable label, especially in case sensitivity is an important item. In our case, sensitivity (i.e. signal to noise) is more important and preferably β-galactosidase is used as a label. The advantages of using β-galactosidase labelled secondary antibodies is that it is more stable at pH 7 and less expensive. A suitable substrate is para-aminophenyl-beta-D galactopyranoside.

An example with an ImmuSpeed™ chip according to the invention is presented in the experimental part.

The invention therefore provides a method for immobilisation of a polysaccharide on a carrier, comprising contacting said polysaccharide with an oxidising agent and a polymer comprising at least two amine and/or amide groups to obtain a polysaccharide-polymer complex and coupling said polysaccharide-polymer complex to said carrier, wherein said carrier comprises a polyimide surface (or polyimide foil). Preferably, said carrier is an ImmuSpeed chip. In a preferred embodiment, the polyimide surface (or foil) has been activated by NaOH treatment. The invention further comprises a carrier obtained or obtainable by said method, i.e. a carrier comprising a polyimide surface (or foil) which surface has been provided with a polysaccharide-polymer complex. The binding of said polysacchaide-polymer complex can be covelant as well as non-covalent depending on the amount of desired regeneration cycles.

The second example of a suitable carrier is an amine reactive biosensor (for example provided by ForteBio and described in more detail in WO 2003/004160). Proteins are covelently coupled onto the sensor surface by, amine chemistry techniques which have already been described above. In short, carboxylic groups on the sensor surface are Modified using EDC/NHS to form N-hydroxysuccinimide esters; thus activating the sensor surface. The sensors are then contacted with a polysaccharide-polymer complex (prepared as described herein). The N-hydroxysuccinimide esters react with the amines on the protein surface to form covalent links. Any unreacted NHS-esters are quenched. The association of a protein of interest (for example an antibody directed to Salmonella) is now measured in a label-free, quantitative and real-time way by using polarization interferometry (also referred to as biolayer interferometry). Biolayer interferometry uses non-diffractive optics to interrogate and resolve the size and density of a biomolecular layer at a solid-solution interface in real-time. Like in SPR biosensing, changes in the effective refractive index, caused by the formation of thin films at the solid-solution interface, such as the binding of molecules to the biosensor tip, result in changes in the sensing reflected light. The effective index of the reference wave remains unaffected, but the phase of the sensing wave will be changed. The wavelength shifts can be measured and correlated with layer thickness and density. Changes in the conformation of the layer after hybridisation can also be assessed, but here, only the specific binding of anti-Salmonella antibodies is probed.

In the studied biosensor configuration of the so-called Octect (ForteBio), single-use biosensors with an optical coating layer are prepared with antigen preferably externally of the device. In a microtitre plate configuration, sensor surfaces are namely dipped in solutions containing reagents, antigens for immobilization and washing buffers. The optical surface, which is a two-dimensional rounded binding layer, will be loaded with protein-LPS antigens, which can then interact with antibodies from a surrounding solution. Following the necessary incubations externally and/or internally of the machine with samples, the obtained biolayer thicknesses in the eight biosensors are assessed simultaneously in the Octect instrument using the biolayer interferometry as detection system. In this way, analysis of 96 samples in 20 min could be achieved. It should be remarked here that this approach is non-destructive with no cross-over contamination (disposable biosensors), and, therefore, almost the complete sample volume can be retrieved for another analysis series using another series of disposable biosensors assaying another analyte.

One of the advantages of the Octect versus the earlier described Biacore biosensor, is that the amount of serogroups that can be tested at the same time can be increase to for example 5 (for example B, C1, C2, D and E).

The experimental part describes an example with this kind of carrier.

The invention therefore provides a method for immobilisation of a polysaccharide on a carrier, comprising contacting said polysaccharide with an oxidising agent and a polymer comprising at least two amine and/or amide groups to obtain a polysaccharide-polymer complex and coupling said polysaccharide-polymer complex to said carrier, wherein said carrier comprises an amine reactive biosensor. Preferably, said carrier is an Octet-like biosensor. The invention further comprises a carrier obtained or obtainable by said method, i.e. a carrier comprising an amine reactive biosensor that has been provided with a polysaccharide-polymer complex.

The obtained/described carriers can be used in different types of analysis, such as bacteriology (direct assay) or serology (indirect assay).

An example of a serological assay is a method for determining the presence of n antibody directed to an antigen of a gram-negative bacteria in a sample, comprising contacting said sample with a carrier or a biosensor as described above and determining whether the carrier has bound any antibody (FIG. 1). Such a method is for example very suitable for determining the presence of an antibody directed against an O antigen and thus it is indirectly established whether an infection is present or whether a recent infection has occurred. Such a method is for example used to screen slaughter animals for salmonella or to screen animals for salmonella before they are exported abroad. Moreover, the method is also applied to samples obtained from living (for example, farm or zoo) animals.

Examples of samples that can be used in such a method are tissue sample, body fluid, secretes or excretes and more detailed examples are blood, blood derived samples, tissue, meat juice, milk, egg, fluids from an eye, saliva or faeces. As already outlined the samples can be obtained from dead as well as living animals.

A method according to the invention is not limited to a certain immunoglobulin (sub)type but can in principle be every (iso)type immunoglobulin such as (s)IgA₁, (s)IgA₂, IgD, IgG₁, IgG₂, IgG₃, IgG₄, IgM, IgY. Moreover, it may also be any other antigen-binding material. Preferably, such an antigen-binding material is a biomarker of a (history of an) infection.

Such a serological assay is for example directed to one particular serogroup-representing carbohydrate or to different (i.e. multi analyte) serogroup-representing carbohydrates and hence such a method is for example used to determine the presence or absence of a certain salmonella (sub)type.

An example of a bacteriological assay is a method for determining the presence of a gram-negative bacterium in a sample, comprising contacting said sample with a predetermined amount of antibodies directed against an antigen of said bacterium and determining the amount of antibodies not bound to said bacterium with a carrier or a biosensor as described above.

Preferably the antigen is a serogroup-representing carbohydrate.

This method optionally further comprises the removal of non-bound antibodies from contacted sample and predetermined amount of antibodies by for example washing or immuno-magnetic separation procedures, centrifugation or filtering.

For this type of analysis every type of sample can be used, such as animal feed, manure, feathers, soil, water for consumption or sewage water, meat, orange juice, chocolate, skin, vegetables etc. Animal samples may be obtained from living as well as dead animals.

In this bacteriological assay a single type of antibody as well as a mixture of at least two different types of antibodies (directed against different antigens, for example two different serogroup-representing carbohydrates) is used.

Preferably, such serological and bacteriological assays are performed such that the binding to said carrier or said biosensor is determined by Plasmon Surface Resonance or fluorescent microsphere or bead counter.

The source of the samples is as already outlined above unlimited and may for example be obtained from a human or an animal. Examples of suitable animals are (race) horses, pigs, poultry (for example chicken, turkey, quail, duck, and goose), ruminants (for example calf or cow, goat, sheep). The animals may be farm animals, zoo animals as well as free living animals. Moreover, samples from these animals may be obtained from living as well as dead animals.

In yet another embodiment the invention provides a method for determining the presence of a gram-negative bacterium in a sample comprising

contacting said sample with target bacteria-specific, bacteriophages and allowing the bacteriophages to infect said sample

removing non-bound and/or non-invading bacteriophages resulting in a bacteriophage infected sample

bringing the bacteriophage infected sample into contact with an indicator organism susceptible for the used bacteriophages

incubate during at least one bacteriophage multiplication cycle

recover the bacteriophages to obtain a bacteriophage-containing sample

analyse said bacteriophage-containing sample with a carrier or a biosensor according as described above.

Most analytical methods require prior enrichment and growth in specific media to detect bacteria, including salmonella. Usually sample preparation is very time-consuming relatively to the total analysis time. It generally takes 3 to 5 days before the presence of e.g. salmonella can be confirmed. In many situations, this time for analysis is unacceptable and hinders trade and indirectly threats community health.

The objective of this part of the invention is development of a fast (preferably within 24 h) and/or cost-effective and/or specific and/or sensitive diagnostic method for the determination of the presence of micro organisms. For this reason, the development of a biomolecular interaction assay (BIA) which exploits the ability of genus- and/or serovar-specific bacteriophages to multiply in their ‘victim’ bacteria, is aimed. An increment in number of the target pathogen-specific phage(s) indicates not only the presence of the target organism but is also a (semi-)quantitative measure for the content of target bacteria in the tested sample.

A schematic overview of the proposed BIA method is depicted in FIG. 2. A particulate sample is homogenised for example using a Stomacher. Liquid samples are mixed by vigorous shaking. Analyte cells are then extracted or enriched by any suitable method and may comprise (a combination of) selective growth, centrifugation, filtration and/or immuno-magnetic separation (IMS). Enriched cells are fortified with target bacteria-specific bacteriophages and incubated for a few minutes while mixing. Before the multiplication cycle of the bacteriophage is complete, cells are washed to remove as complete as possible any non-bound and non-invading bacteriophages. Following the multiplication cycle of the bacteriophage, the sample is brought in contact with an indicator organism susceptible, i.e. in a life phase that is sensitive for bacteriophage penetration and intracellular multiplication, for the used bacteriophage, preferably at the highest possible concentration (for example concentrated overnight culture). The bacteriophage-bacterium suspension is incubated for at least one bacteriophage multiplication cycle. The phage-infected suspension is then centrifuged or filtered to precipitate/remove cellular material and to recover multiplied bacteriophages. The bacteriophage-containing sample is injected over an LPS-conjugated biosensor chip (according to the invention) to retain these particles in the detector for the generation of analyte-specific biosensor response.

To gain as much time as possible the indicator organism can be kept as a continuous culture in the lab and has a cell density of usually 10⁹ CFU/ml. Such a suspension may be concentrated to 10¹⁰ CFU/ml, as higher cell densities will increase sensitivity of the proposed method.

This method can be used to determine a single type of serovar but to detect multiple serovars in one run, a mix of different bacteriophages and a mixture of possibly different indicator bacteria may have to be applied.

Target bacteria-specific bacteriophages are described in the prior art and examples are provided in the experimental part, for example anti-Salmonella enteritidis bacteriophages.

Phages have been described to attach to LPS, including the phage described in the experimental part for salmonella detection. Suitable carriers/chips are carriers/chips with LPS or with immobilised bacterial surface molecules (thus including membrane proteins and other biomolecules or a combination thereof). Use of LPS of cell membrane material will circumvent the generation of poly- or monoclonal antibodies. If attachment of the phages to bacterial biomolecules (LPS) is not satisfactory in the BIA, biosensor chip-immobilised anti-phage antibodies may have to be used in a successful BIA to capture bacteriophages from the probed sample.

The invention furthermore provides a kit with components suitable for use in any of the described applications. Depending on the customer's demand, such a kit comprises a ready-for use carrier/chip obtained by a method according to the invention. When the customer wants to prepare the carrier himself, the kit will at least comprise (lipo)polysaccharide fortified/enriched with protein (for example haemoglobin or serum albumin) in a predetermined amount, an amount of oxidizing agent (for example periodate), suitable buffers. Optionally, such a kit comprises means for desalting, for example a desalting column. When the customer wants to mix (lipo)polysaccharide and protein himself these components are delivered separately together with an instructions manual. Optionally, such a kit may furthermore comprise positive and/or negative reference sera, a sample dilution buffer and any necessary instruction manual.

The methods as described above are particularly suitable for screening samples on a large-scale basis. In one of the earlier (slow) settings 96 samples were checked within 33 minutes. In a large-scale setting with relative slow biosensor equipment 15.000 samples were screened within 3 months. This number could have been much higher but unfortunately one of the slaughterhouses stopped participating.

The invention will be explained in more detail in the following description, which is not limiting the invention.

### EXAMPLES

### Example 1

### Materials and Methods

### 1.1 Materials

### 1.1.1 Chemicals

Amine coupling kits, consisting of N-hydroxysuccinimide (NHS), 1-ethyl-3-(3-dimethlylaminopropyl)carbodiimide hydrochloride (EDC) and ethanolamine hydrochloride-sodium hydroxide pH 8.5 and the running buffer (HBS-EP), containing 10 mM HEPES, 150 mM sodium hydrochloride, 3 mM EDTA and 0.005% (v/v) surfactant P20 at pH 7.4, were bought from Biacore AB (Uppsala, Sweden), which also supplied ready-to-use 10 mM glycine and 50 mM sodium hydroxide. Ethanol, ethylene glycol, sodium chloride, sodium hydroxide and trichloroacetic acid (TCA) were purchased from Merck (Darmstadt, Germany). Carboxymethylated-dextran sodium salt, sodium cyanoborohydride and carbohydrazide were obtained from Fluka Chemie GmbH (Buchs, Switzerland). CHAPS (Plus one) was delivered by Pharmacia Biotech (Uppsala, Sweden). Sodium acetate trihydrate and acetic acid were supplied by J.T. Baker (Deventer, The Netherlands). Guanidine hydrochloride was obtained from Calbiochem (San Diego, Calif., U.S.A.). Porcine haemoglobin (Hb) and myoglobin (Mb), chicken ovalbumin (Ob); 98% grade V), bovine serum albumin (BSA; 96% Fraction V), sodium periodate, Tween-20, Tween-80 and Triton X-100 were acquired from Sigma Chemical Company (St. Louis, Mo., U.S.A.). Water was obtained from of a Milli Q water purification system (Millipore, Bedford, Mass., U.S.A.).

### 1.1.2 Materials

NAP-5 columns (0.5 ml; Sephadex G-25) were purchased from Amersham Biosciences (Roosendaal, The Netherlands) and were used as described by the producer. CM5 biosensor chips were bought from Biacore AB. Dialysis bag (Spectra/Por) with a cut-off of 1 kDa was obtained from Spectrum Laboratories Inc. (Rancho Dominguez, Calif., U.S.A.)

### 1.1.3 Anti-Salmonella Antisera

The following salmonella monovalent ‘O’ somatic lapine antisera were used: anti-O4, anti-O5, anti-O6, 7, anti-O8, anti-O9, anti-O10, anti-O12, O Poly E (anti-O3, anti-O10, anti-O15, anti-O19, anti-O34). In addition, salmonella polyvalent ‘O’ somatic (Poly A-S) lapine antisera (anti-O2, anti-O3, anti-O4, anti-O5, anti-O6, 7, anti-O8, anti-O9, anti-O10, anti-O11, anti-O12, anti-O13, anti-O15, anti-O16, anti-O17, anti-O18, anti-O19, anti-O20, anti-O21, anti-O22, anti-O23, anti-O28, anti-O30, anti-O34, anti-O35, anti-O38, anti-O40, anti-O41) was used as well. The sera were purchased from Pro-Lab diagnostics (Salmonella Reference Section of the Central Veterinary Laboratory, Weybridge, U.K.). Serogroup specific murine anti-B (anti-O4, O5 en O27), anti-C (anti-O7, O8), anti-D (anti O9, Vi) and anti-E (anti-O3, O19) monoclonal antibodies were bought from SIFIN (Berlin, Germany’).

Sera were diluted 1:20 (v/v) in HBS-EP containing 1.0 M sodium chloride, 1% (m/v) carboxymethylated dextran and 0.05% (v/v) Tween 80, except anti-O5 serum was diluted 1:200 (v/v) and the anti-serogroup specific preparations were diluted 1:100 (v/v) in the same solvent.

### 1.1.4 Reference Avian and Porcine Sera

All reference sera were obtained from the Dutch Animal. Health Service (Deventer, The Netherlands). The obtained avian reference sera were reactive with Salmonella enteritidis (serogroup D₁), S. typhimurium (serogroup B), S. pullorum/gallinarum (serogroup D₁) and S. infantis (serogroup C₁), and were further referred to as C-Se, C-St, C-Spg and C-Si, respectively. These chicken sera were originally prepared for ELISA analyses as positive references. In addition, specific pathogen-free chicken serum (further referred to as C-SPF) was purchased as a negative control reference sample. These sera were reconstituted from freeze-dried material by addition of water at a volume indicated by the manufacturer. C-Se, C-Spg and C-Si were diluted 1:200 (v/v) in HBS-EP containing 1.0 M sodium chloride, 1.0% (m/v) carboxymethylated dextran and 0.05% (v/v) Tween-80, whereas C-SPF and C-St were diluted 1:50 (v/v) in the same solution. Likewise, porcine sera from animals challenged with S. typhimurium and S. livingstone (serogroup C₁) were referenced as P-St and P-Sl, respectively. In addition, Actinobacillus pleuropneumoniae serotype 2-reacting porcine serum used as control in a complement fixation test, was exploited as negative control for porcine serum in the salmonella biosensor assay. The porcine sera were diluted 1:20 (v/v) in HBS-EP containing 1.0 M sodium chloride, 1% (m/v) carboxymethylated dextran and 0.05% (v/v) Tween 80 as end concentrations.

### 1.1.5 Salmonella Stock

The bacteria Salmonella goldcoast (Sg; serogroup C₂), S. livingstone (Si) and S. melaegridis (Sm; serogroup E₁) were obtained from an in-house collection, while S. enteritidis #23 phage type Pt4 (Se), and S. typhimurium X-193 phage type 507 (St) were kind gifts of F. G. van Zijderveld (Animal Sciences Group, Lelystad, The Netherlands). The bacteria were grown in overnight cultures in Nutrient Broth #2 (Oxoid, Basingstroke, U.K.). Stocks of salmonella strains were morphologically and biochemically confirmed as salmonella and also verified for the presence of the correct, expected O-antigens by an agglutination reaction of the cells with specific standard anti O-antigen anti-sera (Pro-Lab diagnostics) as indicated in Table 3 on a glass plate. After addition of a half of the original volume with glycerol (Merck), stocks were stored in portions at −80° C.

TABLE 3
O-antigen verification of the salmonella serovars used for LPS production.
The expected reaction of anti sera used for verification by agglutination, is given

| Salmonella | Agglutination sera | | | | | | | | |
|---|---|---|---|---|---|---|---|---|---|
| Serovar | α-O4^{a} | α-O5 | α-O6, 7 | α-O8 | α-O9 | α-O10 | α-O12 | poly A-S | Poly E |
| S. enteritidis | − | − | − | − | + | − | + | + | − |
| (O9, O12)^{b} | | | | | | | | | |
| S. goldcoast | − | − | + | + | − | − | − | + | − |
| (O6, 8) | | | | | | | | | |
| S. livingstone | − | − | + | − | − | − | − | + | − |
| (O6, 7) | | | | | | | | | |
| S. meleagridis | − | − | − | − | − | + | − | + | + |
| (O3, O10) | | | | | | | | | |
| S. typhimurium | + | + | − | − | − | − | + | + | − |
| (O4, O5, O12) | | | | | | | | | |

^{a}α-O4: antibodies reacting with antigen structure coded with O4; in a similar way the antibodies against O5, O6, 7, O9, O10 and O12 are indicated.
^{b}O antigens specific for salmonella serovar is indicated in brackets

### 1.2 Methods

### 1.2.1 Extraction of LPS

Overnight cultures of salmonella were prepared by applying 100 μl from their corresponding stocks on each of the 120 plates containing brain heart infusion agar (BHIa, Oxoid). The presence of the expected salmonella serovar was confirmed through conventional selective growth, bio- and immunochemical classification, whenever new stock suspensions were produced. The bacteria were harvested from the surface of the plates into 1 ml 9 g/l NaCl (saline) solution per agar plate using a trigalski spatula. Each plate was washed twice with 2 ml saline solution. Bacteria were collected in six centrifugation tubes. Each tube was complemented with 100 ml saline and mixed before centrifugation at 10,000 g and 4° C. for 15 min and supernatant was discarded. This centrifugation step was repeated twice by suspending cells in 75 ml saline wash solution per tube each run. While kept on ice, pelleted bacteria were suspended in water at a volume ratio, which was a 5-fold to the weight of the bacteria. An equivolume of 0.250 M (Se) or 0.500 M (Sg, Sl, Sm and St) TCA was added to give end concentrations of 0.12 M and 0.25 M, respectively, followed by continuous stirring at 4° C. for 3 h. A lipopolysaccharide (LPS)-containing supernatant was then acquired at 20,000 g and 4° C. for 30 min. The pH of the supernatant was adjusted to pH 6.5 with 5 M sodium hydroxide and when nearing the aimed pH with 0.10 M sodium hydroxide. The final volume of the LPS-containing solution was determined prior to storage at −18° C. for 30 min. The solution was diluted with a double volume of freezing cold absolute ethanol from a −18° C. storage place, and incubation was continued overnight at −4° C. without stirring in a closed, in house-build device with circulating cold ethylene glycol/water (1:4, v/v). An LPS-containing pellet was obtained after centrifugation at 20,000 g and −4° C. for 30 min. The particulate material was suspended in a volume of 0.5 ml water per gram original bacterial mass weighed at the start of extraction process. The suspension was dialyzed in a 1-kDa dialysis bag against water at 4° C. for two days with regular intermittent refreshment of the water. The bag content was centrifuged at 20,000 g and at 4° C. for 30 min, and the supernatant was lyophilized. The lyophilisate was weighed to establish the recovery of LPS. LPS was reconstituted in water to make up an end concentration of 5 mg/ml. Dependent of type of LPS and batch (see also section 1.2.2), a volume of 1 mg/ml porcine haemoglobin (Hb) was added to a concentration as indicated in the text. Each batch was portioned into 0.5-mg LPS fractions, which were dried using a vacuum evaporator and then stored at 5-8° C.

### 1.2.2 Optimal Haemoglobin Content

Protein was added to an LPS preparation prior to its chemical modification and immobilization to a sensor chip to acquire high coating levels and high serum responsive antigens. The optimum Hb content in each LPS batch was established by comparison of the responses of immobilized LPS that was fortified with Hb at different levels, using a panel of positive and negative reference sera.

### 1.2.3 Oxidation of LPS

A portion of 0.5 mg haemoglobin-fortified LPS was dissolved in 500 μl 100 mM sodium acetate pH 5.5. Following the addition of 20 μl 50 mM sodium periodate, the solution was incubated for 40 min on ice protected from light. The oxidation of LPS was quenched and the solution was desalted by passing 500 μl of the reaction mixture through an NAP-5 cartridge with a gravity-controlled flow. Modified LPS was eluted with 1 ml 10 mM sodium acetate, pH 4.0. Prior to use, the cartridge was conditioned thrice with 3 ml 10 mM sodium acetate, pH 4.0.

### 1.2.4 Immobilization of LPS

To immobilize the antigens to a sensor chip, the following handlings were conducted at a flow rate of 5 μl/min in a Biacore 3000 instrument controlled by Biacore 3000 Control Software (version 3.1.1; Biacore). Immobilization of oxidized LPS was achieved by execution of the aldehyde-coupling procedure described in BIAapplications Handbook, version AB (1998). Briefly, the dextran layer at the biosensor chip CM5 was activated with a 7-min pulse of a mixture of EDC/NHS available from the amine-coupling kit. The activation was immediately followed by injection of 5 mM aqueous carbohydrazide for 7 min as well.

Deactivation of the excess of reactive groups was then accomplished with a pulse of 1 M ethanolamine for 7 min. Prior to immobilisation of the antigen, LPS was diluted in sodium acetate pH 4.0 in a ratio dependent of the salmonella serovar (see text) and immobilised for 32 min. The linkage between dextran-matrix and antigen was then stabilized by injection of 100 mM sodium cyanoborohydride solved in 10 mM sodium acetate at pH 4 at a flow rate of 2 μl/min for 20 min. A relative response indicative for a successful LPS immobilisation procedure is 2 kRU for a 62.5 μg/ml LPS solution containing 15% (m/m) protein, and 9 kRU for a 250 μg/ml LPS solution containing 50% (m/m) protein.

### 1.2.5 SPR Biosensor Assay

Optical SPR biosensor assays were performed on a Biacore 3000 SPR biosensor platform controlled by the same software as described above. Prior to injection, sera were diluted in HBS-EP buffer containing 1.0% (m/v) carboxymethylated-dextran sodium salt, 1.0 M sodium chloride and 0.05% (m/v) Tween 80 at a ratio of 1:50 (v/v) or otherwise as indicated in the text. The mixtures were incubated for at least 2 min at ambient temperature. Pig sera were injected for 2 min at 40 μl/min, whereas bird sera were injected for 2 min at 5 μl/min or 20 μl/min as indicated.

Regeneration of the chip to recover the antigenic activity of the sensor surface was achieved with a 15-s pulse of 6 mM glycine at pH 2, containing 6 M guanidine hydrochloride, 0.1% (m/v) CHAPS, and 0.1% (v/v) of each Tween-20, Tween-80 and Triton X-100. This was followed with a second regeneration step with the running HBS-EP buffer enriched with 0.05% (m/v) CHAPS (end concentration) for 12 s at 100 μl/min.

### 1.2.6 Monosaccharide Analysis

Trimethylsilylated (methyl ester) methyl glycosides were prepared from the glycan samples by methanolysis (1.0 M methanolic HCl, 24 h, 85° C.) followed by re-N-acetylation and trimethylsilylation, and then analyzed by gas chromatography/mass spectrometry as described [Kamerling J P, Vliegenthart JFG (1989)]. The quantitative analysis was carried out by gas chromatography on a capillary EC-1 column (30 m×0.32 mm, Alltech) using a Chrompack CP 9002 gas chromatograph operated with a temperature program from 140° C. to 240° C. at 4° C./rain, and flame-ionization detection. The identification of the monosaccharide derivatives was confirmed by gas chromatography/mass spectrometry on a Fisons Instruments GC 8060/MD 800 system (Interscience) equipped with an AT-1 column (30 m×0.25 mm, Alltech).

### Results

### LPS Isolation

For the production of LPS, yields of bacterial cells and of LPS were compared for agar plate culture and growth of salmonella in broth (Table 4). For laboratory technical reasons, it was decided to harvest bacteria from agar plates, rather than isolation of the cells from culture flasks. The results of the isolation of LPS from Se, Sg, Sl, Sm and St are summarized in Tables 5 to 9, respectively. The standardized isolation of well-defined LPS is determinative for a successful and robust serological assay. To secure assay performance, batch-to-batch differences should be kept to a minimum. For this reason, several batches of LPS extracted from each Se, Sg, Sl, Sm and St were produced. The recovery of LPS largely depended on the final TCA concentration in the mixture during extraction of LPS (cf. Table 6 and Table 8), although this relationship was not completely clear for the extraction of LPS from St (Table 9). Indeed, no accurate optimal TCA concentration could be determined for each LPS type through the testing of a broad range of TCA concentrations. Here, optimal TCA would yield highest LPS amounts, and give highest specific serological and lowest aspecific biosensor responses. In this study, the TCA concentration chosen as ‘optimal’ for LPS extraction from the different salmonella serotypes was based on the final LPS yields after dialysis, and were 0.12 M, 0.25 M, 0.25 M, 0.25 M and 0.25 M as end concentrations for Se, Sg, Sl, Sm and St, respectively.

TABLE 4
Recovery of LPS from S. enteritidis cells grown either as a
suspension in a bioreactor containing so-called nutrient
broth#2 (broth) or on BHI agar plates (agar). LPS was
isolated using indicated TCA end concentrations. The yield of
LPS relative to the amount of isolated cells is indicated in the
last column.

| LPS | | | | | |
|---|---|---|---|---|---|
| Batch | Culture | | bacteria yield | recovered | LPS yield |
| code | method | TCA (M) | (g) | LPS (mg) | (%, m/m) |
| Se01 | Broth | 0.25 | 3.9 | 16 | 0.42 |
| Se02 | Broth | 0.25 | 5.0 | 21 | 0.41 |
| Se03* | Agar | 0.25 | 14 | 0.2 | 0.00 |
| Se04a | Broth | 0.25 | 3.4 | 0.4 | 0.01 |
| Se04b | broth | 0.5 | 3.4 | 2 | 0.06 |
| Se05** | agar | 0.5 | 7.6 | 2.4 | 0.03 |
| Se06a | agar | 0.5 | 8.8 | 3.9 | 0.04 |
| Se06b | agar | 0.25 | 7.6 | 9.5 | 0.12 |
| Se06c | agar | 0.125 | 8.9 | 13 | 0.14 |
| Se07a | agar | 0.1 | 9.1 | 12 | 0.13 |
| Se07b | agar | 0.05 | 9.2 | 2.9 | 0.03 |
| Se07c | agar | 0.025 | 9.3 | 4 | 0.04 |
| Se2003.1 | agar | 0.125 | 29 | 48 | 0.16 |
| Se2003.2 | agar | 0.125 | 17 | 25 | 0.15 |
| Se2003.4 | agar | 0.125 | 13 | 5.1 | 0.04 |
| Se2005.1 | agar | 0.25 | 15 | 18 | 0.13 |

*pH of TCA-containing mixture is outlying
**some material was lost during sample work up process.

TABLE 5
Recovery of Salmonella enteritidis cells grown on BHIa plates. LPS
was isolated using 0.12 M TCA end concentration (cf. Table 4).
Rec, recovered.

| LPS | Total | | bacteria | |
|---|---|---|---|---|
| Batch | bacterial | Number of | per plate | Rec. LPS/cells |
| (Se) | yield (g) | BHIa plates | (g) | (% m/m) |
| Se2003.1 | 29.09 | 98 | 0.29 | 0.16 |
| Se2003.2 | 17.27 | 60 | 0.28 | 0.15 |
| Se2003.4 | 13.00 | 40 | 0.32 | 0.04 |
| Se2005.1 | 44.5 | 120 | 0.37 | 0.12 |

TABLE 6
Recovery of Salmonella goldcoast cells grown on BHIa plates. LPS
was extracted using a TCA end concentration as indicated.
Rec, recovered.

| LPS | Total | Number of | bacteria | | Rec. |
|---|---|---|---|---|---|
| Batch | bacterial | BHIa | per plate | TCA^{a} | LPS/cells |
| code | yield (g) | plates | (g) | (M) | (% m/m) |
| Sg2003.1 | 40.39 | 120 | 0.34 | 0.075 | 0.01 |
| Sg2003.2 | 35.09 | 120 | 0.29 | 0.25 | 0.41 |
| Sg2003.3 | 12.89 | 40 | 0.32 | 0.25 | 0.36 |
| Sg2005.1 | 46.99 | 120 | 0.39 | 0.25 | 0.30^{b} |

^{a}end concentration TCA in extraction mixture.
^{b}approximately a third of the production was lost during work-up.

TABLE 7
Recovery of Salmonella livingstone cells grown on BHIa plates. LPS
was extracted using 0.250 M TCA end concentration. Rec, recovered.

| LPS | | | | Rec. |
|---|---|---|---|---|
| Batch | Total bacterial | Number of | bacteria per | LPS/cells |
| code | yield (g) | BHIa plates | plate (g) | (% m/m) |
| Sl2003.1 | 32.89 | 120 | 0.27 | 0.52 |
| Sl2003.2 | 13.63 | 40 | 0.34 | 0.51 |
| Sl2005.1 | 47.40 | 120 | 0.40 | 0.64 |

TABLE 8
Recovery of Salmonella meleagridis cells grown on BHIa plates.
LPS was isolated using a TCA end concentration as indicated.
Rec, recovered.

| | Total | Number | Bacteria | | Rec. |
|---|---|---|---|---|---|
| LPS Batch | bacterial | of BHIa | per plate | TCA^{a} | LPS/cells |
| code | yield (g) | plates | (g) | (M) | (% m/m) |
| Sm2003.1a^{b} | 9.10 | 30 | 0.30 | 0.250 | 0.32 |
| Sm2003.1b^{b} | 10.13 | 30 | 0.34 | 0.125 | 0.19 |
| Sm2003.1c^{b} | 10.00 | 30 | 0.33 | 0.075 | 0.06 |
| Sm2003.2^{b} | 40.42 | 120 | 0.33 | 0.075 | 0.02 |
| Sm2003.3 | 37.28 | 138 | 0.27 | 0.250 | 0.47 |

^{a}end concentration TCA in extraction mixture.
^{b}batches Sm2003.1 to 2003.2 were combined to a single batch called Sm2003.1

TABLE 9
Recovery of Salmonella typhimurium cells grown on BHIa plates.
LPS was isolated using 0.250 M TCA end concentration.
Rec, recovered.

| | Total | Number of | bacteria | | Rec. |
|---|---|---|---|---|---|
| Batch | bacterial | BHIa | per plate | TCA^{a} | LPS/cells |
| code | yield (g) | plates^{a} | (g) | (M) | (% m/m) |
| St2003.1 | 28.51 | 69 | 0.41 | 0.125 | 0.18 |
| St2003.2^{b} | 39.21 | 120 | 0.33 | 0.250 | 0.06 |
| St2003.3^{b} | 16.6 | 56 | 0.30 | 0.125 | 0.09 |
| St2003.4a^{b} | 18.15 | 60 | 0.30 | 0.250 | 0.18 |
| St2003.4b^{b} | 19.2 | 60 | 0.32 | 0.125 | 0.06 |
| St2005.1 | 46.80 | 120 | 0.39 | 0.250 | 0.19 |

^{a}end concentration TCA in extraction mixture;
^{b}batches St2003.2 to St2003.4b were combined to a single batch called St2003.2

The monosaccharide composition of isolated LPS preparations were analyzed to reveal the consistency of the isolation and purification procedure for LPS from different salmonella growths. It must be noted that analyses were performed on LPS preparations that were ready for oxidation and for that reason fortified with Hb at levels that were determined most optimal for the LPS batch tested (see below). For this purpose, GC-FID and GC-MS analyses were carried out after methanolysis of the Hb-fortified LPS preparations (Table 10 through Table 14). These results show that Hb does not contribute to a significant amount of carbohydrates in the final LPS preparation. Analysis of BHIa, showed the presence of exclusively galactose (Gal) and glucose (Glc). The content of these monosaccharides was 5.6 μl/mg dried BHIa. Analyses of the salmonella LPS preparations, demonstrated the occurrence of Gal, Glc, N-acetyl glucosamine (GlcNAc), glycero-manno-heptose (Hep), 2-keto-3-deoxy-octonic acid (KDO), mannose (Man) and rhamnose (Rha; 6-deoxy-mannose) in accordance with their carbohydrate structures. Their relative occurrence was expressed as a molar ratio relative to 1.0 Man (as part of the PS region) or relative to 3.0 Hep (as part of the core region). It should, however, be noted that the core region contains 2 or 3 Hep residues. Furthermore, GlcNAc can originate from either GlcNAc as in the repeating unit of Sl LPS, or from glucosamine (GlcN), which occurs as disaccharide in the lipid A moiety as backbone for the attached lipids. Gal occurs in the core region, which is conserved in all Salmonella enterica serovars, and in the PS region of Se, Sg, St and Sm as well. In these cases, the molar ratio of Gal is expected to be in excess of 1.0 Man, except for Sg in which each repeating unit contains 2 Man residues. The monosaccharide analyses did not include the detection of O-acetylated, posphoryl-ethanolaminated or phosphorylated constituents, nor that of abequose (Abe; 3,6-dideoxy-xylohexose) or tyvelose (Tyv; 3,6-dideoxy-arabinose), which occur in the polysaccharide and core regions of the isolated LPS types as well.

Analysis of Se LPS, showed the occurrence of Gal, Man and Rha at a molar ratio of 1.4, 1.0 and 1.2, respectively, in batch Se2003.1, whereas this ratio was 1.1, 1.0 and 0.9, respectively, in batch Se2003.4 (Table 5). This ratio is in good compliance with the composition of a repeating unit as [Tyv-]Man-Rha-Gal, except the Rha ratio was significantly too high in batch Se2003.1. The carbohydrate content calculated on the basis of determined monosaccharides, was significant higher in batch Se2003.4, namely 241 μg compared 123 μg of batch Se2003.1. Considering the occurrence of 2 GlcN residues in the lipid A and a single GlcNAc residue in the core region and a single Man residue in each repeating unit, the number of repeating units was estimated 19 and 20 in batches Se2003.1 and Se2003.4, respectively.

Monosaccharide analysis of oxidized Se2003.1 clearly demonstrates significant differences with the non-oxidized identical batch (Table 10). In contrast to the two GlcN residues, it is expected that the non-reducing, terminal GlcNAc residue is for the greater part oxidized. Alditol derivatives were not detected by the monosaccharide analysis applied and a corresponding amount of GlcNAc-ol was not determined. As Gal and Man in the repeating unit are not susceptible towards periodate oxidation, the molar ratio of Man in the oxidized batch is normalized to that of Man in the non-oxidized batch. It should be noted that both Gal residues in the core region are susceptible towards oxidation and thus the total Gal ratio is affected. Inspection of the molecular structure of Se LPS, suggests that in addition to terminal GlcNAc and core Gal, terminal KDOII-KDOIII disaccharide, and conjugated HepI and terminal HepIII are susceptible to periodate oxidation as well. Indeed, the molar ratios of these monosaccharide residues suggest the loss of one Hep residue and approximately 1.6 KDO residues. It should be noted that KDOII may not be completely oxidized when this residue is conjugated with a posphoryl-ethanolamine group.

TABLE 10
Monosaccharide analysis of S. enteritidis LPS and of oxidized
S. enteritidis LPS. LPS was fortified with Hb at 15% (m/m).
Molar ratios were determined on the basis of two GlcN and one
GlcNAc residues (detected as three GlcNAc residues) present in the
core and lipid A regions (referred to as CORE) and on the basis one
Man residues in the repeating unit (referred to as UNIT). Normalized
GlcNAc and Man residues are indicated by underlining. Carbohydrate
content was determined in 0.5 mg LPS preparations, except
monosaccharide analysis was performed on 125 mg
oxidized material.

| | Molar ratio | | | | | |
|---|---|---|---|---|---|---|
| | | | Batch Se2003.1 | | | |
| | Batch Se2003.1 | | (oxidized) | | Batch Se2003.4 | |
| Monosaccharide | CORE | UNIT | CORE | UNIT | CORE | UNIT |
| Gal | 29.7 | 1.4 | 28.7 | 1.3 | 26.3 | 1.1 |
| Glc | 6.3 | 0.3 | 7.5 | 0.4 | 6.9 | 0.3 |
| GlcNAc | 3.0 | + | 2.2 | + | 3.0 | + |
| Hep | 2.8 | + | 1.9 | + | 2.0 | + |
| KDO | 3.2 | + | 1.4 | + | 2.3 | + |
| Man | 21.6 | 1.0 | 21.6 | 1.0 | 23.5 | 1.0 |
| Rha | 24.8 | 1.2 | 24.4 | 1.1 | 20.9 | 0.9 |

| | | | |
|---|---|---|---|
| Carbohydrate | 123.0 | —^{b} | 241 |
| content (μg)^{a} | | | |
| Nr of repeating | 19 | — | 20 |
| units | | | |

^{a}does not include the contribution of Tyv residues;
^{b}Amount of LPS-containing material analysed was not accurately determined.

Compared to Se LPS, monosaccharide analysis of Sg LPS suggests that LPS structure were smaller as the number of repeating units was significant lower (Table 11). The relative contribution of core Gal to PS Gal is for that reason larger and total molar ratio is found 1.5. In a similar way, the molar ratio for Glc is found at 1.3 (batch Sg2003.2) and 1.5 (batch Sg2003.3), whereas the molar ratio for Rha fits with the expected structure. Batch Sg2003.2 seems however to contain less terminal HepIII and terminal KDOIII and could therefore offer less possibility for immobilization to the sensor chip.

TABLE 11
Monosaccharide analysis of LPS isolated from S. goldcoast fortified
with Hb at 50% (m/m). Molar ratios were determined on the basis of
two GlcN and one GlcNAc residues (detected as three GlcNAc residues)
present in the core and lipid A regions (referred to as CORE) and on the
basis two Man residues in the repeating unit (referred to as UNIT).
Normalized GlcNAc and Man residues are indicated by underlining.
Carbohydrate content was determined in 0.5 mg LPS preparations.

| Molar ratio | |
|---|---|
| Batch | Batch |
| Sg2003.2 | Sg2003.3 |

| Monosaccharide | CORE | UNIT | CORE | UNIT |
|---|---|---|---|---|
| Gal | 15.3 | 1.5 | 14.8 | 1.5 |
| Glc | 13.5 | 1.3 | 14.3 | 1.5 |
| GlcNAc | 3.0 | + | 3.0 | + |
| Hep | 2.9 | + | 2.3 | + |
| KDO | 3.0 | + | 2.8 | + |
| Man | 20.5 | 2.0 | 19.2 | 2.0 |
| Rha | 10.6 | 1.0 | 10.2 | 1.0 |

| | | |
|---|---|---|
| Carbohydrate content | 170 | 199 |
| (μg)^{a} | | |
| Nr of repeating units | 9 | 8 |

^{a}does not include the contribution of Abe residues.

Normalisation of the number of core residues from the monosaccharide analysis results of Sl LPS (Table 12) was hampered by the occurrence of GlcNAc in the repeating units of the PS. When the number of Hep residues was set at 3.0, an unacceptable overestimation of the number of KDO residues arose. For that reason, the number of Hep was set at 2.0, but may need to be modified, so that the number of KDO is closer to 3.0. As the number of Man residues in each repeating unit is four, molar ratios were corrected for 4.0 Man residues. On the basis of a molar ratio of 4:1 of Man/GlcNAc in the PS region, the number of repeating units was calculated on the basis of the remaining core GlcNAc and Lipid A GlcN residues. This calculation revealed that the number of repeating units in Sl was also relatively small, namely 8 and 10 units in batch Sl2003.1 and batch2003.2, respectively.

TABLE 12
Monosaccharide analysis of LPS isolated from S. livingstone
fortified with Hb at 50% (m/m). Molar ratios were determined on the
basis of two Hep residues present in the core (referred to as CORE) and
on the basis four Man residues in the repeating unit (referred to as
UNIT). Normalized GlcNAc and Man residues are indicated by
underlining. Carbohydrate content was determined in 0.5 mg LPS
preparations.

| Molar ratio | |
|---|---|
| Batch | Batch |
| Sl2003.1 | Batch Sl2003.2 |

| Monosaccharide | CORE | UNIT | CORE | UNIT |
|---|---|---|---|---|
| Gal | 3.5 | 0.4 | 5.1 | 0.4 |
| Glc | 17.4 | 1.7 | 23.5 | 1.6 |
| GlcNAc | 14.0 | 1.4 | 18.7 | 1.3 |
| Hep | 2.0 | 0.2 | 2.0 | 0.14 |
| KDO | 2.7 | 0.3 | 2.7 | 0.2 |
| Man | 40 | 4.0 | 57 | 4.0 |
| Rha | n.d. | n.d. | n.d. | n.d. |

| | | |
|---|---|---|
| Carbohydrate content | 212 | 239 |
| (μg) | | |
| Nr of repeating units | 8 | 10 |

n.d., not detected.

TABLE 13
Monosaccharide analysis of S. meleagridis LPS. Batches Sm2003.1
and Sm2003.3 were fortified with 50% (m/m) Hb. Molar ratios were
determined on the basis of two GlcN and one GlcNAc residues (detected
as three GlcNAc residues) present in the core and lipid A regions
(referred to as CORE) and on the basis one Man residues in the repeating
unit (referred to as UNIT). Normalized GlcNAc and Man residues are
indicated by underlining. Carbohydrate content was determined in 0.5
mg LPS preparations.

Molar ratio

| | |
|---|---|
| Batch | Batch |
| Sm2003.1 | Sm2003.3 |

| Monosaccharide | CORE | UNIT | CORE | UNIT |
|---|---|---|---|---|
| Gal | 20.4 | 1.5 | 22.4 | 1.4 |
| Glc | 7.9 | 0.6 | 4.8 | 0.3 |
| GlcNAc | 3.0 | + | 3.0 | + |
| Hep | 2.8 | + | 2.9 | + |
| KDO | 2.8 | + | 2.9 | + |
| Man | 14.2 | 1.0 | 15.4 | 1.0 |
| Rha | 16.1 | 1.1 | 17.6 | 1.2 |

| | | |
|---|---|---|
| Carbohydrate content | 170 | 220 |
| (μg)^{a} | | |
| Nr of repeating units | 12 | 13 |

^{a}does not include the contribution of O-acetyl groups, which may be attached to the repeating Gal residues.

Monosaccharide analysis of Sm LPS (Table 13) showed a completely different composition as that for Sl LPS in accordance with its molecular structure containing Man-Rha-Gal repeating units. As described above, the molar ratio for Gal is more than the expected 1.0 in the repeating unit partly by the contribution of Gal residues in the core region.

Likewise, equimolar ratios are expected for Glc, Man, Rha and Gal as these residues form a repeating unit in St LPS (Table 14). It should be noted here that abequose is also part of the repeating unit, but is not in the analysis applied. Batch St2003.2, however, contains less oxidizable Hep and KDO, which may affect the efficacy of the immobilization of LPS from this preparation. At the other hand, Batch St2003.2 contains much more carbohydrate than batch St2003.1, namely 249 μg relative to 154 μg in 0.5 mg LPS, respectively.

TABLE 14
Monosaccharide analysis of S. typhimurium LPS. Batches
St2003.1 and St2003.2 were fortified with Hb at 15% (m/m) and 25%
(m/m), respectively. Molar ratios were determined on the basis of two
GlcN and one GlcNAc residues (detected as three GlcNAc residues)
present in the core and lipid A regions (referred to as CORE) and on
the basis one Man residues in the repeating unit (referred to as UNIT).
Normalized GlcNAc and Man residues are indicated by underlining.
Carbohydrate content was determined in 0.5 mg LPS preparations.

| Molar ratio | |
|---|---|
| Batch St2003.1 | Batch St2003.2 |

| Monosaccharide | CORE | UNIT | CORE | UNIT |
|---|---|---|---|---|
| Gal | 24.7 | 1.4 | 24.2 | 1.4 |
| Glc | 17.0 | 1.0 | 16.2 | 1.0 |
| GlcNAc | 3.0 | + | 3.0 | + |
| Hep | 2.8 | + | 2.6 | + |
| KDO | 2.7 | + | 2.4 | + |
| Man | 20.5 | 1.0 | 19.2 | 1.0 |
| Rha | 19.9 | 1.1 | 19.5 | 1.2 |

| | | |
|---|---|---|
| Carbohydrate content | 154 | 249 |
| (μg)^{a} | | |
| Nr of repeating units | 15 | 14 |

^{a}does not include the contribution of (O-acetylated) Abe residues.

### Protein-Supported Immobilization of LPS

Unexpectedly, intact LPS poorly coupled through its KDO-carboxylic acid function to EDC/NHS-activated carboxymethated dextran, and no signal responses of reference sera were observed. To improve immobilization of LPS to the sensor chip, LPS was oxidized using sodium periodate to create reactive aldehyde groups in its carbohydrate constituents, which would allow the so-called aldehyde coupling procedure, i.e. condensation of aldehyde with a hydrazide function into a hydrazone linkage followed by reduction hydrazide product. Without oxidation, LPS had indeed little potential to immobilize to a surface of a CM5 chip coated with carbohydrazide (results not shown).

Coupling of oxidized LPS, however, gave disappointing reactivity with reference sera, probably as a result of insufficient immobilization of the antigens. Commercially acquired phenol-extracted LPS, either intact or detoxified (i.e. cleavage of lipid A), from S. enteritidis gave low responses when immobilized after oxidation, namely 187 RU and 167 RU, respectively. It must be noted, however, that besides poor coupling, oxidation may have destroyed a part of the antigenic structures, which may give poor serological responses. The degree of oxidation was investigated by monosaccharide analysis of Se LPS (Table 10). This analysis revealed that relative amounts of KDO, Hep and GlcNAc, which are constituent of the core region and not of the repeating antigenic units in the PS part, were significantly reduced compared to non-oxidized Se LPS. Importantly, monosaccharide residues part of the PS, and thus antigenic structures, apparently remained intact under the mild oxidation conditions, which were applied.

TABLE 15
Biosensor responses following immobilization and responses of
reference antisera flowed over chip surfaces, which were prepared with
oxidized and non-oxidized Se or St LPS in the presence of 15% (m/m) porcine
Hb.

| | | Biosensor response (RU) | | | | | |
|---|---|---|---|---|---|---|---|
| Type of | Periodate | Level of | | | | | |
| LPS | treatment | immobilization | α-O4^{a} | α-O5 | α-O9 | α-O12 | O poly A-S |
| Se | No | 6515 | 1 | 9 | 6 | 9 | 4 |
| St | No | 4402 | 2 | 156 | 1 | 5 | 0 |
| Se | yes | 4265 | −21 | −3 | 77 | 202 | 159 |
| St | yes | 5950 | 302 | 5005 | −12 | 225 | 137 |

^{a}anti-serum against indicated O antigen was tested

TABLE 16
Biosensor responses following immobilization and responses of
reference antisera flowed over chip surfaces, which were prepared with
oxidized St LPS (batch St2003.1) in the presence of 7% (m/m) of the indicated
protein.

| | Biosensor response (RU) | | | | | | | | | | | |
|---|---|---|---|---|---|---|---|---|---|---|---|---|
| Protein | Level of | | | | | | O poly | | | | | |
| added | immob.^{a} | O4 | O5 | O9 | O12 | O poly E | A-S | C-SPF | C-St | C-Se | C-Si | C-Spg |
| BSA^{b} | 344 | n.d^{c} | n.d | n.d | n.d | n.d | n.d | n.d | n.d. | n.d. | n.d. | n.d. |
| BSA | 7450 | 19 | 216 | 1 | 18 | 4 | 14 | 5 | 18 | 21 | 12 | 42 |
| Hb | 3410 | 319 | 3874 | 4 | 192 | 3 | 203 | 11 | 151 | 140 | 51 | 762 |
| Mb | 815 | 59 | 773 | 3 | 45 | 2 | 47 | 8 | 46 | 34 | 20 | 152 |
| Ob | 5540 | 76 | 926 | 10 | 56 | 12 | 56 | 12 | 47 | 45 | 25 | 177 |

^{a}level of immobilization;
^{b}BSA was added to oxidized and desalted LPS;
^{c}immobilization of LPS was considered too low for further reference sera analysis.

To optimize the binding of LPS and consequently improve detection of binding antibodies from sera, oxidation of LPS was then executed in the presence of a protein to allow the formation of protein-LPS complexes through Schiff-base reactions between proteinaceous amines and aldehyde functions of LPS. Indeed, commercially available TCA extracted Se LPS, containing considerable amounts of bacterial proteins, gave improved immobilization at 562 RU compared to phenol-extracted and ion-exchange chromatography-purified Se LPS at 208 RU.

Oxidation of LPS was necessary, as mixtures containing non-oxidized LPS and protein show relatively high immobilization levels but insignificant specific responses (Table 15). In addition, protein addition was only beneficial prior to oxidation of LPS, as addition of BSA to oxidized and desalted St LPS gave acceptable immobilization levels but no expected serological responses (Table 16). In a similar way, Hb yielded relatively high immobilization levels but no serological responses, as expected (results not shown).

For method improvement purposes, proteins with relatively high degree of homology of their primary and secondary structure between homeothermic vertebrate species and occurring in serology-suitable matrices were selected for further investigations. For that reason, the performance of chicken ovalbumin, porcine haemoglobin, bovine serum albumin or porcine myoglobin fortified (7%, m/m) St LPS was compared (Table 16). Haemoglobin gave clearly best improvement of immobilization levels together with best expected antigen-antibody reactivity profile. In the presence of Hb, in particular, O12, poly O A-S and C-St reference sera gave better responses.

This experiment was repeated with the addition of BSA, Hb and Mb at levels indicated in Tables 17 to 20 using batches Se2005.1, Sg2005.1, S12005.1 and St2005.1. This time, O4 and O5 bound to immobilized St LPS as expected (Table 20). Evaluation of these results summarized in Tables 16 to 20 revealed that when considering all expected responses simultaneously per LPS type, the addition of Hb gave highest specific responses compared to the addition of BSA and Mb. Furthermore, in most cases standard deviations that occurred with Hb as supportive protein, were more favorable than those for the addition of BSA and Mb. Haemoglobin was, therefore, selected for further experimentation.

It was observed that the O poly A-S anti sera probably contains a low anti-serogroup C₁ and C₂ titers, as in the case of testing immobilized Sg LPS (Table 18) and Si LPS (Table 19) relatively low responses are found.

TABLE 17
Biosensor responses in response units (RU) following
immobilization and responses of reference antisera flowed over chip surfaces,
which were prepared with Se LPS oxidized in the presence of 15% (m/m) of
the indicated protein. Values were corrected for the C-SPF responses, which
are listed as well. Standard deviations are indicated in brackets (N = 5, except
for chicken and swine sera N = 4).

| | | Antiserum tested | | | | | | | | |
|---|---|---|---|---|---|---|---|---|---|---|
| | Immob. | | | O poly | Anti- | | C- | C- | P- | C- |
| protein | level^{a} | O9 | O12 | A-S | serogroup D | C-Se | St | Spg | St | SPF |
| Hb | 3403 | 72 | 268 (1) | 160 (2) | 187 (3) | 197 (6) | 55 | 1753 | 76 | 87 |
| | | (4) | | | | | (7) | (5) | (13) | |
| Mb | 4228 | 47 | 242 (3) | 133 (5) | 158 (4) | 167 (9) | 40 | 1674 | 80 | 107 |
| | | (4) | | | | | (12) | (6) | (16) | |
| BSA | 4883 | 31 | 185 (2) | 99 (16) | 126 (5) | 126 (11) | 28 | 1277 | 68 | 74 |
| | | (11) | | | | | (30) | (4) | (24) | |

^{a}level of immobilization.

TABLE 18
Biosensor responses in response units (RU) following
immobilization and responses of reference antisera flowed over chip surfaces,
which were prepared with Sg LPS oxidized in the presence of 50% (m/m) of
the indicated protein. Values were corrected for the C-SPF responses, which
are listed as well. Standard deviations are indicated in brackets (N = 5, except
for the chicken and swine sera N = 4).

| | | Antiserum tested | | | | | |
|---|---|---|---|---|---|---|---|
| | Immob. | | | | Anti- | | |
| protein | level^{a} | O6, 7 | O8 | O poly A-S | serogroup C | P-Sl | C-SPF |
| Hb | 5542 | 318 (1) | 249 (11) | 69 (3) | 145 (10) | 52 (17) | −1 |
| Mb | 9880 | 237 (1) | 211 (12) | 45 (7) | 81 (26) | 162 (34) | 0 |
| BSA | 10344 | 196 (4) | 124 (8) | 19 (24) | 61 (15) | 110 (22) | −6 |

^{a}level of immobilization.

TABLE 19
Biosensor responses in response units (RU) following
immobilization and responses of reference antisera flowed over chip surfaces,
which were prepared with Sl LPS oxidized in the presence of 50% (m/m) of the
indicated protein. Values were corrected for the C-SPF responses, which are
listed as well. Standard deviations are indicated in brackets (N = 5, except for
chicken and swine sera N = 4).

| | | Antiserum tested | | | | | |
|---|---|---|---|---|---|---|---|
| | Immob. | | | Anti- | | | |
| protein | level^{a} | O6, 7 | O poly A-S | serogroup C | C-Si | P-Sl | C-SPF |
| Hb | 8180 | 125 (21) | 16 | 167 (4) | 377 (12) | 46 (7) | −17 |
| Mb | 10819 | 97 (24) | 8 | 115 (0) | 349 (3) | 96 (26) | −24 |
| BSA | 11364 | 24 (5) | −33 | 40 (5) | 200 (2) | 68 (20) | 76 |

^{a}level of immobilization

TABLE 20
Biosensor responses in response units (RU) following
immobilization and responses of reference antisera flowed over chip surfaces,
which were prepared with St LPS oxidized in the presence of 15% (m/m) of the
indicated protein. Values were corrected for the C-SPF responses, which are
listed as well. Standard deviations are indicated in brackets (N = 5, except for
chicken and swine sera N = 4).

| | | Antiserum tested | | | | | | | | |
|---|---|---|---|---|---|---|---|---|---|---|
| | Immob. | | | | O poly | Anti- | | C- | | C- |
| protein | level^{a} | O4 | O5 | O12 | A-S | serogroup B | C-St | Spg | P-St | SPF |
| Hb | 3355 | 419 | 494 | 212 | 107 (1) | 809 (5) | 398 | 619 | 272 | 9 |
| | | (3) | (7) | (2) | | | (14) | (24) | (5) | |
| Mb | 2826 | 353 | 432 | 176 | 80 (9) | 722 (2) | 356 | 532 | 246 | 17 |
| | | (8) | (4) | (8) | | | (20) | (24) | (14) | |
| BSA | 5588 | 388 | 304 | 158 | 82 (16) | 542 (4) | 324 | 324 | 244 | 4 |
| | | (7) | (7) | (16) | | | (24) | (16) | (17) | |

^{a}level of immobilization.

Immobilization levels and expected reactivity of agglutination sera with S. enteritidis LPS (batch Se2003.1), S. goldcoast (batch Sg2003.2), S. livingstone (batch Sl2003.1), S. typhimurium (batch St2003.1) and S. meleagridis (Batch Sm2003.1) revealed a correlation with relative amount of Hb added before oxidation (see for example FIG. 3). It was also found that for a salmonella serovar-specific LPS type, the optimum Hb concentration was also production batch dependent.

On guidance of maximum response of the expected antigenic profile using a panel of standard and reference control sera and on guidance of low responses from avian SPF reference sera, optimum Hb concentration was determined for each type and for each batch of LPS (Table 21). In the text, oxidized, haemoglobin-containing LPS preparations are further referred to as LPS^{ox}-Hb preparations.

It should be noted that, in any experiment, immobilization level did not correlate with serological responses but correlated with the amount Hb that was added.

TABLE 21
Effect of haemoglobin and LPS concentration on the final
immobilization level of LPS derived from S. enteritidis (Se)
and S. typhimurium (St).

| LPS | Se LPS | | St LPS | |
|---|---|---|---|---|
| concentration | 10% (m/m) | | 10% | 15% |
| (μg/ml) | Hb^{a} | 15% (m/m) Hb | (m/m) Hb | (m/m) Hb |
| 25 | | | | 9197 |
| 62.5^{b} | 2194-2065 | 3173 | | |
| 62.5^{b} | 3400-3000 | | | |
| 62.5^{b} | | | 8414 | |
| 62.5^{b} | 2374 | | | |
| 125 | 3940-3647 | | | |
| 250 | 3370 | | 10645 | |

^{a}Concentration of Hb relative to LPS
^{b}prepared on separate sensor channels.

The effect of dilution of Se LPS and St LPS before oxidation in the presence of 10% (m/m) or 15% (m/m) Hb relative to LPS, respectively, was investigated (Table 21). These results did not clearly reveal a correlation between LPS concentration and coupling level.

TABLE 22
Relationship between immobilization level of LPS^{ox}-Hb and
serologic responses of O antigen antiserum and avian control sera. Fields for
which a serological response was expected are shaded in a green color and
values within the field are underlined. Control sera were diluted in HBS-EP
containing 0.5 M sodium chloride and 0.5% (m/v) carboxymethylated dextran.

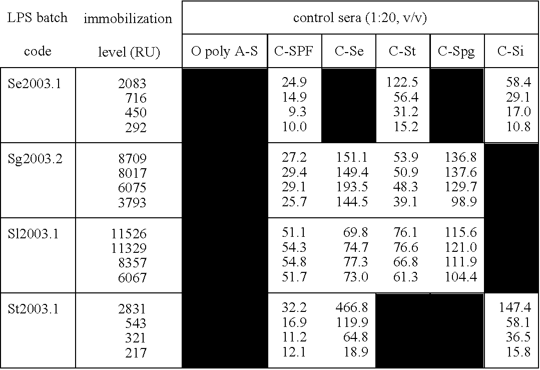

### LPS Stability and Robustness of Immobilization

Reproducibility and repeatability of the immobilization of LPS and the specific response of reference sera were tested. The Se, Sg, Sl and St LPS preparations were oxidized in the presence of their corresponding optimal Hb concentration, desalted, and then stored in solution at 4° C. in the dark. Under identical conditions, but accounting with the variability generally observed for immobilization of Molecules at a biosensor surface, immobilization levels of oxidized LPS were either comparable in the cases of the Sl (5% RSD) and St (8% RSD) batches or tended to increase in the cases of the Se (13% RSD) and Sg (8% RSD) batches over two months of storage (Table 23 through Table 26). The responses of reference sera were probed on the prepared biosensor chips as well. Inspection of these did not reveal a correlation between immobilization level and specific response. For example, immobilization levels of Se LPS and Sg LPS may tend to increase over time; this increase was not reflected in the response of the binding of serum antibodies to the immobilized antigens. The relative standard deviation varies between 12% and 38%. When considering the first 3 measuring days (day 0, day 7 or 10, and day 14 or 17) the variance is greatly reduced from 3.5% to 23% (results not shown). Tables 27 to 30 summarizes the repeatability of the method at several moments over a 12-months period. At each analysis time point, a fresh aliquot of Hb-fortified LPS was oxidized, immobilized and analysed and thus reflects the sum of variability of several steps.

TABLE 23
Response of agglutination and reference anti-sera with LPS^{ox}-Hb
Se2003.1 prepared at day 0 and stored at 5-8° C.. The LPS
preparation was immobilized and tested after oxidation at the
days indicated.

| | | Se2003.1 oxidized in presence of 15% Hb | | | | |
|---|---|---|---|---|---|---|
| Day of | immobilization | | | | | C-Spg |
| analysis | level (RU) | O9 | O12 | O poly A-S | C-Se | (1:100) |
| 0 | 2708 | 446 | 302 | 330 | 2597 | 3535 |
| 10 | 3007 | 352 | 258 | 252 | 2205 | 3726 |
| 17 | 3194 | 472 | 320 | 292 | 2520 | 3586 |
| 31 | 3642 | 509 | 356 | 340 | 1486 | 3727 |
| 62 | 3640 | 361 | 223 | 156 | 1676 | 2329 |
| Average | 3238 | 428 | 292 | 274 | 2097 | 3381 |
| St. dev. | 407 | 69 | 52 | 74 | 498 | 594 |
| RSD (%) | 13 | 16 | 18 | 27 | 24 | 18 |

TABLE 24
Response of agglutination and reference anti-sera with LPS^{ox}-Hb
Sg2003.2 prepared at day 0 and stored at 5-8° C. The LPS preparation
was immobilized and tested after oxidation at the days indicated.

| | | Sg2003.2 oxidized | | |
|---|---|---|---|---|
| Day of | immobilization | in presence of 50% Hb | | |
| analysis | level (RU) | O6, 7 | O8 | O poly A-S |
| 0 | 7262 | 668 | 465 | 117 |
| 7 | 9972 | 714 | 531 | 110 |
| 14 | 10597 | 657 | 566 | 111 |
| 20 | 11453 | 857 | 502 | 100 |
| 28 | 11866 | 1085 | 504 | 130 |
| 59 | 12002 | 484 | 364 | 58 |
| Average | 10525 | 744 | 489 | 104 |
| St. dev. | 869 | 226 | 77 | 27 |
| RSD (%) | 8 | 30 | 16 | 26 |

TABLE 25
Response of agglutination and reference anti-sera with LPS^{ox}-Hb
Sl2003.1 prepared at day 0 and stored at 5-8° C. The LPS preparation
was immobilized and tested after oxidation at the days indicated.

| | | |
|---|---|---|
| | | Sl2003.1 oxidized in |
| Day of | immobilization | presence of 50% Hb |

| analysis | level (RU) | O6, 7 | O poly A-S |
|---|---|---|---|
| 0 | 11509 | 318 | 84 |
| 7 | 13581 | 289 | 63 |
| 14 | 14870 | 250 | 54 |
| 20 | 13582 | 475 | 65 |
| 28 | 14976 | 468 | 75 |
| 59 | 14744 | 202 | 31 |
| Average | 13877 | 334 | 62 |
| St. dev. | 707 | 127 | 17 |
| RSD (%) | 5 | 38 | 27 |

TABLE 26
Response of agglutination and reference anti-sera with LPS^{ox}-Hb
St2003.1 prepared at day 0 and stored at 5-8° C.. The LPS preparation was
immobilized and tested after oxidation at the days indicated.

| | | St2003.1 oxidized in presence of 15% Hb | | | | | | |
|---|---|---|---|---|---|---|---|---|
| Day of | immobilization | | O5 | | | | | C-Spg |
| analysis | level (RU) | O4 | (1:200) | O12 | O poly A-S | C-St | C-Se | (1:100) |
| 0 | 5391 | 594 | 540 | 278 | 362 | 441 | 544 | 1634 |
| 10 | 4432 | 463 | 567 | 242 | 273 | 448 | 356 | 1442 |
| 17 | 4473 | 517 | 578 | 250 | 262 | 362 | 396 | 1294 |
| 31 | 4769 | 559 | 582 | 296 | 326 | 440 | 255 | 1464 |
| 62 | 4996 | 442 | 321 | 209 | 196 | 340 | 297 | 983 |
| Average | 4812 | 515 | 518 | 255 | 284 | 406 | 370 | 1363 |
| St. dev. | 397 | 64 | 111 | 34 | 64 | 51 | 112 | 244 |
| RSD (%) | 8 | 12 | 22 | 13 | 22 | 13 | 30 | 18 |

TABLE 27
Responses of freshly oxidized Se LPS (batch Se2003.1) on indicated
time points (in months). The LPS was isolated from bacterial cells,
fortified with 15% (m/m) Hb, dried and stored at 4-7° C.
until day of oxidation, immobilization and analysis.

| | | Se2003.1 oxidized in presence of 15% Hb | | | | |
|---|---|---|---|---|---|---|
| | | | | | C-Se | C-Spg |
| Analysis | immobilization | | | O poly | (1:200, | (1:100, |
| (month) | level (RU) | O9 | O12 | A-S | v/v) | v/v) |
| 0 | 2708 | 446 | 302 | 330 | 2597^{a} | 3535^{b} |
| 1^{c} | 1326 | 252 | 165 | 172 | 464 | 1274 |
| 2 | 2380 | 372 | 262 | 230 | 502 | 1478 |
| 3 | 2003 | 398 | 208 | 230 | 676 | 1864 |
| 5 | 2309 | 144 | 207 | 337 | 534 | 1539 |
| 7 | 3487 | 240 | 444 | 547 | 786 | 2368 |
| 9 | 3721 | 215 | 407 | 576 | 705 | 2415 |
| 12 | 1450 | 78 | 145 | 219 | 159 | 1272 |

^{a}serum was diluted 1:50 (v/v)
^{b}serum was diluted 1:100 (v/v)
^{c}LPS was diluted at another volume ratio

TABLE 28
Responses of freshly oxidized Sg LPS (batch Sg2003.2) on indicated
time points (in months). The LPS was isolated from bacterial cells,
fortified with 50% (m/m) Hb, dried and stored at 4-7° C.
until day of oxidation, immobilization and analysis.

| | | Sg2003.2 oxidized | | |
|---|---|---|---|---|
| Analysis | immobilization | in presence of 50% Hb | | |
| (month) | level (RU) | O6, 7 | O8 | O poly A-S |
| 0 | 7262 | 668 | 465 | 117 |
| 1 | 7428 | 929 | 451 | 100 |
| 2 | 9023 | 639 | 459 | 91 |
| 3 | 13152 | 474 | 606 | 119 |
| 5 | 8087 | 549 | 446 | 294 |
| 7 | 9724 | 622 | 382 | 286 |
| 9 | 8870 | 692 | 508 | 364 |
| 12 | 7088 | 491 | —* | 281 |

TABLE 29
Responses of freshly oxidized S1 LPS (batch Sl2003.1) on indicated
time points (in months). The LPS was isolated from bacterial cells,
fortified with 50% (m/m) Hb, dried and stored at 4-7° C.
until day of oxidation, immobilization and analysis.

| | | |
|---|---|---|
| Analysis | immobilization | Sl2003.1 |

| (month) | level (RU) | O6, 7 | O poly A-S |
|---|---|---|---|
| 0 | 11509 | 318 | 84 |
| 1 | 11442 | 417 | 62 |
| 2 | 12280 | 287 | 55 |
| 3 | 13152 | 231 | 65 |
| 5 | 11896 | 217 | 159 |
| 7 | 11563 | 271 | 149 |
| 9 | 10882 | 298 | 191 |
| 12 | 10138 | 204 | 131 |

TABLE 30
Responses of freshly oxidized St LPS (batch St2003.1) on indicated
time points (in months). The LPS was isolated from bacterial cells, fortified
with 15% (m/m) Hb, dried and stored at 4-7° C.. until day of oxidation,
immobilization and analysis.

| | | St2003.1 oxidized in presence of 15% Hb | | | | | |
|---|---|---|---|---|---|---|---|
| Analysis | immobilization | | O5 (1:200, | | O poly | | C-Spg (1:200, |
| (month) | level (RU) | O4 | v/v) | O12 | A-S | C-St | v/v) |
| 0 | 5391 | 594 | 540* | 279 | 362 | 441 | 1634^{a} |
| 1^{b} | 3487* | 339 | 470 | 181 | 207 | 352 | 597 |
| 2 | 4623 | 482 | 325 | 257 | 242 | 411 | 612 |
| 3 | 4079 | 425 | 608 | 187 | 235 | 982 | 624 |
| 5 | 6873 | 359 | 369 | 167 | 302 | 393 | 605 |
| 7 | 5410 | 681 | 734 | 334 | 453 | 559 | 837 |
| 9 | 4008 | 638 | 664 | 278 | 441 | 524 | 824 |

^{a}serum was diluted 1:100 (v/v);
^{b}Following oxidation, LPS-containing solution was diluted twice instead of once.

### Example 2

### Introduction

SPR Biosensor for Detection of Egg Yolk Antibodies Reflecting Salmonella enteritidis Infections

Salmonella is one of the major causes of bacterial gastro-enteritis of humans (Fischer, 2004; van Duynhoven et al., 2005). In the Netherlands, between 1994-1998, Salmonella enterica serovar enteritidis (S.e.) was the most often isolated serovar (Pelt et al. 1999). Within this serovar, eggs and egg products were the most important source of infection. Despite several control measures, approximately 9% of the Dutch layer flocks become infected annually. As egg contamination with Salmonella continues to be a threat for public health, it is important to detect an infection of a flock as soon as possible by an adequate surveillance programme.

The current Dutch monitoring system in layer finisher hens is based on serology (Bokkers, 2002). The aim is to reduce the prevalence of S.e. and S. typhimurium in the layer sector. Sampling, however, occurs only twice: before and at the end of the laying period. The current surveillance programme, therefore, cannot detect all infections of flocks during the layer period, and farmers cannot ‘guarantee’ that their products are from Salmonella-free layers.

Consequently, the surveillance programme should be improved. As an alternative to current serology, testing of eggs for antibodies could be performed. Egg sampling has the advantage that it can be performed on egg packing plants, in a high sampling frequency and with large sample sizes.

Tests for detection of antibody in eggs have been developed and used before. The existing tests are often based on enzyme-linked immunosorbent assays (ELISA) using different (combinations of antigenic components of Salmonella spp. (see for examples Refs. Gast et al, 2002 and 1997; Skov et al, 2002; Holt et al, 2000; Desmidt et al., 1996; Sachsenweger et al., 1994; Van Zijderveld et al., 1992). Recently, the possible suitability of biosensors for the detection of humoral response has been recognized (Bergwerff and van Knapen, 2006; Bergwerff and van Knapen, 2003; Jongerius-Gortemaker, 2002; Pyrohova et al., 2002; Vetcha et al., 2002; Li et al., 2002; Liu et al., 2001; Uttenthaler et al. 1998). A biosensor consists of a re-usable immobilized biological ligand that ‘senses’ the analyte, and a physical transducer, which translates this phenomenon into an electronic signal (Jongerius-Gortemaker et al., 2002). The use of biosensors promises the possibility of high throughput analyses, and also the detection of multiple serovars or serogroups within a family of infectious disease agents—or antibodies against these agents—in a single run. This offers the opportunity to improve surveillance programmes, as more samples can be tested in a higher frequency during the layer period.

This example evaluates the sensitivity, specificity and discriminatory capacity of a surface plasmon resonance (SPR) biosensor (biacore 3000) antibody detection test in egg yolk based on the lipopolysaccharide (LPS) of Salmonella enterica serovar enteritidis and compares the results to those obtained with a g,m flagellin based commercial ELISA test kit and a LPS based commercial ELISA test kit for detection of egg-antibodies by creating and analyzing receiver operating characteristic (ROC) curves.

### 2. Materials and Methods

### 2.1 SPR Biosensor Method

We used the surface plasmon resonance biosensor (biacore 3000 by Biacore AB, Uppsala, Sweden) detection method of serum antibodies against Salmonella enteritidis using LPS antigen by appolying the invention described herein to egg yolk. After separation of egg yolk and egg white, the egg yolk was diluted 1:5 (v/v) in 10 mM HEPES buffer at pH 7.4, containing 3 mM EDTA, 0.15 M sodium hydrochloride, 0.005% (v/v) surfactant P20 (Biacore AB, Sweden), and additional 0.85 M sodium chloride (Merck, Darmstadt, Germany), 1% (m/v) carboxymethylated dextran (Fluka Chemie, Buchs, Germany) and 0.05% (v/v) Tween 80 (Merck, Germany). It was mixed With glass pearls, centrifuged at 15,000 g at ambient temperature for 25 min; the supernatant was filtrated over a 0.45-μm filter (Schleicher & Schuell, Dassel, Germany).

A second adaptation was the cleaning of the sensor chip. Following analysis of each series of 15 egg yolk samples, a solvent containing 0.5% (w/v) sodium dodecyl sulphate (Biacore AB, Sweden) was injected to remove deposited egg yolk components.

### 2.2 Reference Sera and Egg Yolks

Sera were used as reference in the various tests due to unavailability of sample stock of well-defined reference egg yolks. Lyophilized, defined SPF and reference sera originating from chickens infected with a) S. enteritidis, b) S. typhimurium, c) S. infantis, or d) S. pullorum were obtained from the Animal Health Service Ltd. (Deventer, Netherlands). These sera were prepared from pooled sera. Before use, lyophilized sera were reconstituted in 1 ml Milli-Q. Additionally, monoclonal mouse anti-Salmonella antibody anti-group B, -group C, -group D and -group E was used (Sifin, Berlin, Germany).

Internal-control-egg yolks were used to establish analytical sensitivity and repeatability of the biosensor assay. They consisted of a specific pathogen free (SPF) egg yolk sample (Animal Health Service Ltd., Netherlands) and a highly immuno-responsive pre-ovulatory follicle sample originating from experiment 2 (cf. section 2.4. below).

### 2.3 ELISA

Samples were assayed using sandwich enzyme immunoassay techniques. Two commercially available S.e. antibody detection kits were used; Flockscreen S.e. Guildhay (Guildford, England) and FlockChek S.e. IDEXX (Westbrook, Me., USA). The samples were analyzed according to the company's procedures.

The Guildhay S.e. indirect ELISA is based on LPS as antigen. The wells of microtiter plates were coated with LPS, 1:500 dilutions of samples were added in mono. Test results were expressed as an S/P ratio according to the following formula:

$$S \text{/} P = \frac{( \begin{matrix}optical density sample - \\ optical density negative controls\end{matrix} )}{( \begin{matrix}optical density positive controls - \\ optical density negative controls\end{matrix} )} . \qquad ( 1 )$$

The S/P ratio was interpreted using the following criteria: Egg yolk: S/P≦0.08=immuno-negative; 0.08<S/P<0.25=immuno-suspect; S/P≧0.25 immuno-positive.

The IDEXX S.e. competitive ELISA is based on g,m flagellar antigen. The wells of microtiter plates were coated with g,m flagellar antigen, 1:2 dilutions of samples were added in mono. The results were expressed as S/N ratio as follows: optical density sample

$$S \text{/} N = \frac{optical density sample}{optical density negative controls} \qquad ( 2 )$$

The S/N ratio was interpreted using the following criteria: Egg yolk: S/N≧0.75=immuno-negative; 0.75<S/N<0.59=immuno-suspect; S/N≦0.59=immuno-positive.

### 2.4 Experiments

The egg samples used in this study originated from two infection experiments.

Experiment 1. Fifteen one-week-old layer hens (Isa Brown) were housed in negative pressure high-efficiency particulate air, filter (HEPA) isolators with a volume of 1.3 m³ and fitted with a wire floor of 1.1 m², and applying a 12 h light to 12 h dark photoperiod rhythm. The isolators were ventilated at a rate of approximately 30 m³/h. During the growing period, no Salmonella could be cultured from bedding. The chickens were provided with non-medicated feed and water ad libitum. They were housed, handled and treated following approval by the institutional animal experimental committee of the Dutch Animal Health Service Ltd. in accordance with the Dutch regulations on experimental animals. All hens were inoculated orally once with 1×10⁸ CFU per bird in week 20 of the experiment using S. enteritidis CL344 (Animal Health Service Ltd., Deventer, The Netherlands). Before and after inoculation, eggs were collected on a daily basis, but not labeled individually and not dated. The eggs were stored at ambient temperature for four weeks and subsequently at 4° C. The experiment ended in week 22 and produced 147 ‘positive’ and 71 ‘negative’ samples.

Experiment 2. This experiment is described in detail by Van Eerden et al. (2005, in prep). In short, 128 15-week-old layer hens (Lohmann Brown, 16 birds, 8 replications) were divided into two groups (8 hens each) and housed individually in two climate cells, used as isolators, in the same room, under a 9 h light and 15 h dark photoperiod rhythm. During the growing period until inoculation, no Salmonella was cultured from feces. They were provided with non-medicated feed and water ad libitum. The animal experiment was conducted according to the Guidelines for Animal Experimentation of Wageningen University and approved by the Ethical Committee under Reference Number of 2003219. Each of sixty-four hens (16 birds, 4 replications) was inoculated orally once with 1×10⁸ CFU nalidixic acid-resistant S.e. (ASG, Lelystad, the Netherlands) one week after the experiment started. The other 64 birds were considered uninfected controls. Eggs were collected at day 21 and day 28 post-inoculation. Twelve times, the eggs from one climate cell were collected and pooled after cracking of the shell (cf. section 2.5 below). Four of the pooled egg samples were taken from ‘uninfected’ climate cells. Besides the collection of pooled egg samples, ten egg samples were taken from ten individual birds, of which seven were uninfected. The egg yolk and white were mixed and stored at −20° C. The experiment ended four weeks after inoculation. Pre-ovulatory follicles were then harvested from eight uninfected and five infected individual birds.

### 2.5 Preparation of Egg Samples

Eggs from experiment 1 were prepared in the following manner. To facilitate aseptic preparation, the eggshells were disinfected with a 70% (v/v) aqueous ethanol. Subsequently, the eggs were cracked, and the contents were collected in sterile petri dishes. A volume of 1 ml egg yolk was collected using a sterile disposable syringe and portioned in 200 μl fractions. Each fraction was diluted with a buffer appropriate for either SPR biosensor or ELISA analysis, and then stored at −20° C.

Likewise, pooled egg yolk and white and pre-ovulatory follicles obtained from experiment 2 were fractionated, diluted and stored at −20° C.

### 2.6 Evaluation of the SPR Biosensor Method

### 2.6.1 Analytical Sensitivity and Specificity

To establish the limit of detection of the assay, eight 1:2 (v/v) serial dilutions of a highly immuno-responsive egg yolk sample and a SPF negative control sample were analyzed in triplicate by the SPR biosensor. Analysis of variance (ANOVA) was performed using SPSS (SPSS for Windows, Standard Version, 1999) to evaluate differences in SPR biosensor responses obtained after injection of the serially diluted control samples.

Reference sera were used to spike a SPF yolk for to test the specificity of the SPR biosensor assay. For this purpose, egg yolk was spiked with 1) S. enteritidis-(serogroups D), 2) S. infantis-(serogroups C), 3) S. pullorum-(serogroups D), and 4) S. typhimurium-(serogroups B) reacting antisera. These samples were diluted by their volumes either at a rate of 1:100 (1, 2 and 3) or at 1:50 (4). Further specificity testing was performed by spiking SPF egg yolk with 1:100 (v/v) diluted mouse monoclonal antibody reacting with Salmonella serogroups B, C, D and E.

### 2.6.2 Repeatability

The repeatability of the SPR biosensor assay was assessed by running the highly immuno-responsive egg yolk sample and the SPF negative control egg yolk sample twice on a single day and on three consecutive days (in triplo). Means, standard deviations (SD) and percent coefficient of variation (% CV) values were calculated in Excel 2000 (Microsoft software package).

### 2.6.3 ROC Curves

Receiver operator characteristic (ROC) curves were generated using the results from the SPR biosensor and ELISA analyses to assess the test performances of each assay (Zweig and Campbell, 1993). Using SPSS, the overall accuracy of each assay was calculated from the integrated area under the curve (AUC), corresponding standard error (SE) and the probability of the null hypothesis of the true AUC being 0.5. By use of non-parametric ROC analysis (Metz et al., 1998), the accuracy of SPR biosensor assay detection of antibodies against S.e. was compared with the accuracy of the two ELISA's. The gold standard was the infection status of the experimental group.

### 2.6.4 Diagnostic Sensitivity and Specificity

In a ROC curve the true positive rate (sensitivity) is plotted in function of the false positive rate (100-specificity) for different cut-off points of a parameter. Each point on the ROC curve represents a sensitivity/specificity pair corresponding to a particular decision threshold. Thus, the maximum diagnostic sensitivity at the highest diagnostic specificity for the SPR biosensor assay and the two ELISA's were calculated, using SPSS. For the SPR biosensor test using the samples from experiment 1, the maximum diagnostic specificity at the highest diagnostic sensitivity and, the optimal combined diagnostic sensitivity and specificity were also calculated.

### 3. Results

### 3.1 Analytical Sensitivity and Specificity

A 1:640 (v/v) dilution of the highly immuno-responsive egg yolk sample was, at 50 RU, the highest dilution tested that differed significantly (P<0.001) from the negative control.

The test signal of the SPF egg yolks spiked with S. enteritidis-(1:100, 145 RU), S. pullorum-(1:100, 1012 RU) or S. typhimurium-(1:50, 58 RU) positive sera were above the optimized cut-off value of 52 RU (cf. section 3.4.1 below) and considered positive, as was the SPF egg yolk spiked with mouse anti-Salmonella group D (1:100, 130 RU). Non-spiked SPF yolk was found to be negative, i.e. average response was 30 RU. The yolks spiked with S. infantis-(1:100, 24 RU) positive serum and mouse antiserum against. Salmonella serogroups B (1:100, 27 RU), C (1:100, 16 RU), and E (1:100, 15 RU) were also below the cut-off value.

### 3.2 Repeatability

The coefficient of variation within a single day was 1% for the highly immuno-responsive egg yolk sample and 13% for the negative sample. The coefficient of variation from day-to-day during three days was 2% for the positive sample and 17% for the negative sample.

### 3.3 Threshold Determination

### 3.3.1 ROC Analysis

ROC analysis was performed on the assay results of 71 and 135 egg yolk samples from uninfected and infected chickens, respectively, from experiment 1 (not all tests were performed on 12 samples from infected chickens). Integrated areas under ROC curves were 0.892 (SE 0.024, P<0.001) for the SPR biosensor assay; 0.432 (SE 0.039, P=0.103) for the IDEXX ELISA and 0.430 (SE 0.039, P=0.096) for the Guildhay ELISA (Table 31). The ROC curves are depicted in FIG. 4. The integrated area (AUC), and thus the overall accuracy, for the SPR biosensor assay was significantly larger than those of the IDEXX (Z=11.5, P<0.001) and Guildhay ELISA (Z=10.5, P<0.001).

ROC analysis was also performed for four combined egg white and yolk samples and 15 egg yolk samples from uninfected, and eight combined egg white and yolk samples and eight egg yolk samples infected chickens from experiment 2. The integrated areas under ROC curves were 0.811 (SE 0.082, P 0.002) for the SPR biosensor assay, 0.615 (SE 0.098, P=0.098) for the IDEXX ELISA and 0.870 (SE 0.064, P<0.001) for the Guildhay ELISA (Table 32 and FIG. 5). The AUC of the SPR biosensor assay was significantly (Z=1.9, P=0.055) larger than that of the IDEXX ELISA, but not different from that of the Guildhay ELISA (Z=−1.0, P=0.322).

### 3.4 Performance Estimates

### 3.4.1 Diagnostic Sensitivity and Specificity Estimates

With respect to the results of the samples acquired from Experiment 1, samples from the uninfected population gave biosensor responses ranging from 6 to 50 RU. The responses of the samples from the infected population ranged from 11 to 3584 RU. At a cut-off value of 52 RU, 24 out of 135 samples had to be considered immuno-negative.

A cut-off value of 52 RU yielded the highest possible diagnostic specificity estimate of 100% (with a 95% exact confidence interval (CI) of 95-100%) and a diagnostic sensitivity estimate of 82% (95% CI: 76-98%) for the SPR biosensor assay test. A cut off value of 10 RU yielded the highest possible diagnostic sensitivity estimate of 100% (95% exact CI: 97.400%) and a −specificity estimate of 1% (95% CI: 0-4%). A cut-off value of 42 RU yielded the optimal combined diagnostic sensitivity and −specificity: 84% (95% CI: 77-90%) and 99% (95% CI: 96-100%), respectively:

At a cut-off value of OD₅₅₀ₙₘ 0.11, the IDEXX ELISA had a diagnostic specificity of 100% and a −sensitivity of 1% (95% CI: 0-3%). The OD₅₅₀ₙₘ of the samples from the uninfected population ranged from 0.174 to 1.377, i.e. in excess of the cut off value at 0.11. Of the positive population, 145 out of 147 samples had to considered immuno-negative at the chosen cut-off value, namely corresponding OD₅₅₀ₙₘ ranged from 0.042 to 1.572. The Guildhay ELISA had a diagnostic specificity of 100% and a −sensitivity of 16% (95% CI: 10-22%) at a cut-off value of OD₆₅₀ₙₘ 0.12. None of the samples from the uninfected population showed OD₆₅₀ₙₘ values in excess of 0.12 (0.051 to 0.093). In case of the positive population, 124 out of 147 samples had to be considered immuno-negative. The OD₆₅₀ₙₘ of these samples ranged from 0.048 to 1.471.

In the case of Experiment 2, a cut-off value of 542 RU yielded the highest possible diagnostic specificity estimate of 100% (95% exact CI: 82-100%) and a diagnostic sensitivity estimate of 63% (95% CI: 39-86%) for the SPR biosensor assay test. The samples from the uninfected population had RU values ranging from 101-448. The infected population values ranged from 117-3012 and 6 out of 16 samples had negative test results. At a cut-off value of OD₅₅₀ₙₘ 0.49, the IDEXX ELISA had a diagnostic specificity of 100% and a −sensitivity of 19% (95% CI: 0-38%). None of the samples from the uninfected population had OD₅₅₀ₙₘ values of less than 0.49. The values ranged from 0.537-1.621. Of the positive population, 13 out of 16 samples had negative test results at the chosen cut-off value. The values ranged from 0.134-1.630. The Guildhay ELISA had a diagnostic specificity of 100% and a −sensitivity of 67% (95% CI: 43-91%) at a cut-off value of OD₆₅₀ₙₘ 0.14. None of the samples from the uninfected population had OD₆₅₀ₙₘ values of more than 0.14. The values ranged from 0.072-0.140. Of the positive population, 5 out of 15 samples had negative test results (one sample could not be tested). The values ranged from 0.086-2.144.

### 4. Discussion

The aim of this study was to quantify the test characteristics of the SPR biosensor for the detection of S.e. antibodies in eggs. The results showed that the SPR biosensor assay performed significantly better than the two commercially available ELISA's for samples from Experiment 1 The combined optimal diagnostic sensitivity and −specificity of the SPR biosensor was 84% (77-90%) and 99% (96-100%), respectively. Neither the g,m flagellin-based IDEXX ELISA, nor the LPS-based Guildhay ELISA were able to detect S.e. infection with a higher combined diagnostic sensitivity and specificity using this test panel. This study indicates that an SPR biosensor assay could be a new and powerful tool for monitoring Salmonella enterica serovar enteritidis infections in layer flocks through antibody detection in eggs.

The SPR biosensor assay offers the possibility of detecting infections in fast and reliable way. The high quality of the test and the technical and animal welfare advantages of egg collection are good reasons to explore its use for screening of populations. In addition, the configuration of the applied SPR biosensor from Biacore allows the simultaneous detection of antibodies to multiple Salmonella serovars in a single run in a single sensor channel or in separate sensor channels on the same sensor chip (results not shown). This could be of significance because it is well known that serovars differ over countries and over time (see for examples Refs. Guerin et al., 2005; van Duijnkeren et al., 2002).

The test evaluation was carried out using eggs from two experiments that were not carried out specifically for this test evaluation, possibly influencing test performance. The ‘positive’ eggs were collected probably at a time point that humoral response was developing in the exposed chickens. These ‘premature’ eggs were analyzed and their false-negative results interfere with the evaluation of the assays. Could it have been possible to exclude eggs until 2 weeks post-infection, the diagnostic sensitivity of each test, ELISA or SPR biosensor, would have been improved.

Antibody detection in serum is more sensitive than in eggs, because the appearance of antibodies in eggs is preceded by the appearance in serum by a week (Gast and Beard, 1991; Sunwoo et al., 1996; Skov et al., 2002). However, flock sensitivity of tests for antibodies in eggs can be improved by taking more samples, which is easier when using eggs.

The biosensor performance (AUC 0.892) was compared to that of two commercial ELISA's, (IDEXX AUC 0.432, Guildhay AUC 0.430). To our knowledge the IDEXX ELISA was not validated for eggs, but quantitative data exist about the test's performance in comparison to other tests: Van Zijderveld et al. (1992) evaluated four different ELISA's for diagnosis of S.e. infections in experimentally infected chickens. They reported a specificity of 100% and a sensitivity of 95% for 127 egg yolks from eggs laid between 13 and 40 days after infection with S.e. In our evaluation, the IDEXX test performed not as well as in the 1992 evaluation. An explanation could be the different sample selection, as our samples originated from infection experiments that stopped at 2 and 4 weeks after inoculation, having had less time to develop a humoral response.

Shared O-antigens among members of Salmonella serogroups B and D are known to limit the specificity of detecting S.e. using lipopolysaccharide antigens (de Vries et al., 1998; Baay and Huis in 't Veld, 1993; Hassan et al., 1990). This is confirmed by our results: the assay could not differentiate between infections with serovars enteritidis, gallinarum and typhimurium, sharing O 9 and O 12. As the zoonotic serovars of the three (S. typhimurium plus S. enteritidis) represent 80% of isolates identified by the national reference laboratories participating in the Enter-net surveillance network between 1998-2003 (Fischer et al., 2004), this finding has limited clinical relevance for the human population. The assay did differentiate between SPF egg yolk spiked with mouse anti-Salmonella group B (1:100, 27 RU) and D (1:100, 130 RU). This is not surprising, because the LPS of Salmonella enteritidis has O 1, O 9 and O 12 as somatic antigens, whilst the group specific test reagents contain the following monoclonal antibodies; anti-Salmonella group B: Anti-O 4, O 5, O27; anti-Salmonella group D: Anti-O9.

The cut-off value from Experiment 2 was much higher than the cut-off from Experiment 1, possibly because part of our samples consisted of egg white and yolk instead of egg yolk only.

For different applications, different cut-off values may be Optimal. Relative costs or undesirability of errors (false positive/false negative classifications) and the expected relative proportions of infected and uninfected hens are important parameters in the determination of the cut-off value, which affects the diagnostic value of the assay. We would suggest a cut-off value which minimizes the number of false positive results, reasoning that frequent sampling and testing would be necessary if the assay was to be used in a surveillance programme in the layer population, given the relatively low prevalence of S.e.

The SPR biosensor technique has successfully detected egg antibodies to determine experimental infections in chickens. In future screening programmes, the SPR biosensor could possibly detect different analytes at the same time.

TABLE 31
ROC analysis of the results of samples derived from Experiment 1
analyzed by SPR biosensor, IDEXX and Guildhay ELISA's.

| | SPR | | |
|---|---|---|---|
| | biosensor | | Guildhay |
| Characteristic | assay | IDEXX ELISA | ELISA |
| Optimized cut- | 52 RU | OD_{550 nm} 0.11 | OD₆₅₀ₙₘ 0.12 |
| off | | | |
| Diagnostic | 82 | 1 | 16 |
| sensitivity (%) | | | |
| 95% CI (%) | 76-89 | 0-3 | 10-22 |
| Diagnostic | 100 | 100 | 100 |
| specificity (%) | | | |
| 95% CI^{a} | 95-100 | 95-100 | 95-100 |
| AUC | 0.892 | 0.432 | 0.430 |
| 95% CI | 0.844-0.939 | 0.356-0.508 | 0.355-0.506 |

^{a}Fisher's exact test

TABLE 32
ROC analysis of the results of samples derived from Experiment 2
analyzed by SPR biosensor, IDEXX and Guildhay ELISA's.

| | SPR | | |
|---|---|---|---|
| | biosensor | | Guildhay |
| Characteristic | assay | IDEXX ELISA | ELISA |
| Optimized cutoff | 542 RU | OD_{550 nm} 0.49 | OD₆₅₀ₙₘ 0.14 |
| Diagnostic | 63 | 19 | 67 |
| sensitivity (%) | | | |
| 95% CI | 39-86 | 0-38 | 43-91 |
| Diagnostic | 100 | 100 | 100 |
| specificity (%) | | | |
| 95% CI^{a} | 82-100 | 82-100 | 82-100 |
| AUC | 0.811 | 0.615 | 0.870 |
| 95% CI | 0.649-0.972 | 0.424-0.806 | 0.745-0.996 |

^{a}Fisher's exact test

### Example 3

### Introduction

Direct Detection of Campylobacter Spp. Through Monitoring Bacteriophage Infections Using LPS-Coated Beads

Campylobacter is the most commonly food-borne pathogen in developed countries, causing gastroenteritis characterized by watery and/or bloody diarrhea. Campylobacter is associated with Guillain-Barré (GBS), Reiter's and haemolytic uremic (HUS) syndromes and reactive arthritis (FSAI, 2002; Lake et al., 2003; Tauxe, 2000). In the last 20 years, the infection rate of Campylobacter is still increasing in many developed countries, maybe due to the improvements in detection and reporting. In the United States of America, 2,400,000 cases of campylobacteriosis are reported annually corresponding to approximately 1% of the USA population (Tauxe, 2000).

Wild birds and domestic animals are reservoirs for Campylobacter and shed bacteria to the environment. Poultry is an importance vehicle for Campylobacter infection in humans. Indeed, strains, which were isolated from chickens, could be isolated from patients as well (Coker, 2000). Epidemiological studies have shown that consumption and handling of poultry meat should be considered as a major risk for human infection with C. jejuni or C. coli (FSAI, 2002). The most consistent risk factor in United States, New Zealand and Europe has been consumption or contact with raw or undercooked poultry, accounting for 10% to 50% of all cases of campylobacteriosis (Tauxe, 2000). C. jejuni, C. coli and C. lari represent about 90% of human campylobacteriosis (Stern and Line, 2000). The infective dose of Campylobacter is considered to be low, ranging from 500-10,000 cells (FSAI, 2002).

Campylobacter are Gram negative, curve, S-shaped, or spiral shaped bacilli having one or two flagella at one of the poles and highly motile (Christensen et al., 2001). Campylobacter grows between 30.5° C. and 45° C. at an optimum temperature of 42° C. Optimum growth is established at 10% carbon dioxide, 5-6% oxygen, and 85% nitrogen (FSAI, 2002).

Traditional phenotyping methods for determination of Campylobacter take to 5 days and involve pre-enrichment followed by isolation from selective agar and confirmation by biochemical test. Due to the perishable nature of food items and the speed required for analysis of food products more rapidly, sensitive and specific methods are needed for cost effective Campylobacter detection.

Immunomagnetic separation (IMS) procedures were used by Waller and Ogata (2000), Che et al. (2001), Yu et al. (2001) to concentrate C. jejuni from poultry meat without pre-enrichment cell culture step. This approach could retrieve 10⁴ colony forming units (cfu)/g in poultry meats as detected with atomic force and fluorescence microscopy (Yu et al., 2001). IMS can potentially reduce pre-enrichment time of Campylobacter and may overcome the problems of inhibitors from food sources such as PCR inhibitors (Benoit and Donahue, 2003). The use of IMS may thus speed up the enrichment of the analyte. This example describes a down-stream detection method using Campylobacter-specific bacteriophages, i.e. small viral organisms that attach to or infect living Campylobacter bacteria. Their attachment or infection is dependent of the phase of life cycle of the bacterium. Binding to or infection of Campylobacter may namely occur in the stationary, log or lag phase of the bacterium and depends of the phage species as well. Infection of the bacterium results usually in a high number of copies of the bacteriophage. Recording this increment of phages is therefore used as an analytical instrument to trace the presence of Campylobacter in the original sample.

The aim of this study is to demonstrate the application of bacteriophages as specific and sensitive analytical tools for the detection of Campylobacter in animal products, such as faeces and (poultry) meats.

### Materials and Methods

### Experimental Set-Up

Following homogenisation, e.g. facilitated by stomachering, IMS will be used to purify and concentrate Campylobacter from contaminated samples, such as meat and faeces. In a second step, IMS-isolated bacteria will be incubated with an appropriate strain of bacteriophage. Non-attaching bacteriophages will be washed from the cell isolate using the same IMS procedure. Infected and/or bacteriophage-carrying IMS-immobilised Campylobacter are then introduced in a fresh and pure culture of reference Campylobacter that is in a stationary phase. This cell culture is used as a foreign host to boost the multiplication of the bacteriophages. Following a short culture to allow the bacteria to reach their log-phase, bacteriophages will be harvested by centrifugation. The bacteriophage-containing supernatant will be incubated with LPS-coated fluorescent beads. Here, the bead is coated as described in the Example with the LPS isolated from Campylobacter used as the host organism. The presence of bacteriophages bound to the fluorescent beads will be tested in two ways. Following the addition of and incubation with anti-bacteriophage antibodies tagged with a fluorescent label, the amount of fluorescence will correspond with the concentration of bacteriophages and indirectly with the concentration of Campylobacter in the original sample. In an alternative approach, anti-LPS antibodies containing a fluorescent tag will compete with bacteriophages for binding places. A decrease of recorded fluorescence compared to a Campylobacter-free sample will, therefore, indicate a Campylobacter positive sample.

The test will be validated in terms of selectivity and sensitivity for C. jejuni, C. coli and C. larii in different matrices, including faeces, skin and meat from pigs and chickens. Closely related organisms, such as Arcobacter species, will be used to test the specificity of the method.

### Bacterial and Viral Strains and Culture Condition

C. jejuni (ATCC 33291) and C. coli (ATCC 33559) will be bought from Microbiologics (St. Cloud, USA). The bacteria will grow in tryptone soya broth (TSB) (Oxoid, CM 129, Hampshire, England) for 24 h at 42° C., under microaerophilic atmosphere, which will be generated using a gas package (BBL, Becton Dickinson, Sparks, USA). Campylobacter are then plated onto Charcoal-Cefoperazone-Deoxycholate Agar (mCCDA) (Campylobacter blood-free selective agar base [Oxoid, CM 739] with CCDA selective supplement [Oxoid, SR155], cefoperazone 32 μg/ml and amphotericin B 10 μg/ml) and incubated under microaerophilic atmosphere for 24 to 48 h at 42° C. One colony of pure Campylobacter is then transferred to tryptic soya agar (TSA) (Oxoid, CM131) and will be incubated under microaerophilic atmosphere for 24 to 48 h at 42° C. and will then placed in a refrigerator at 4° C. until use. Campylobacter-infecting bacteriophages NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684 are acquired from the National Type Culture Collection (London, United Kingdom).

### Sample Preparation

The pure Campylobacter culture stored at 4° C. will be subcultured in TSB and incubated under microaerophilic atmosphere for 24 h at 42° C. This is the host for exponential growth of the bacteriophage.

An amount of 25 g of ground chicken fillet will be suspended in 225 ml of Preston broth (Nutrient broth No. 2 [Oxoid, CM 67], 5% (v/v) lysed horse blood [Oxoid, SR48], Campylobacter growth supplement [Oxoid, SR232] and modified Preston Campylobacter selective supplement [Oxoid, SR204]) contained by a stomacher bag. The Preston broth medium will be prepared according to the manufacturer's instruction. The sample-containing stomacher bag will be homogenized thoroughly for 90 s in a stomacher (Interscience, St. Nom, France). The entire suspension will be then be incubated under microaerophilic atmosphere at 42° C. for an appropriate incubation time to allow growth of Campylobacter.

### Immunomagnetic Separation

After enrichment with Preston broth, the stomacher bag containing, the sample will be placed into the incubation pot of the IMS machine (Pathatrix™, Microscience, Cambridgeshire, UK). The apparatus is then Operated according to the instructions of the manufacturer. Briefly, 50 μl of anti-Campylobacter magnetic beads (Microscience) will be added to the sample, which is then recirculated 30 min at 37° C. The magnetically-immobilized beads are released, washed with 100 ml of pre-warmed buffered peptone water (peptone; Becton Dickinson) 10 mg/ml, sodium chloride (Merck, Darmstadt, Germany) 5 mg/ml, disodium hydrogen phosphate dihydrate (Merck) 4.5 mg/ml, potassium dihydrogen phosphate (Merck) 1.5 mg/ml adjusted to pH 7.2) and then drawn to the magnet again. Wash solution was removed leaving a 200 μl suspension for selective growth and bacteriophage analyses.

### Detection of Bacteriophages

Campylobacter-carrying IMS-beads are contacted with a small volume of bacterium-specific bacteriophages. Following a short incubation to allow specific attachment of the phages to the surface of the targeted bacterium, IMS beads are washed and sampled to set a reference point in the final analysis procedure. The rest of the suspension is mixed with a suspension of fresh Campylobacter species to host the growing bacteriopage. Following incubation at 42° C., the suspension is centrifuged and the supernatant will be supplemented with a volume of Campylobacter LPS-coated fluorescent beads. Multiplication of the phages is then assessed following the addition of either fluorescently labelled anti-bacteriophage antibodies or fluorescently labelled anti-Campylobacter antibodies. Following an incubation of 15 min, the beads are analysed using e.g. a BioPlex device (Bio-Rad) to screen fluorescence immobilised on the beads as a result of specific binding reactions.

### Example 4

### Introduction

Detection of Anti-Salmonella Antibodies in Porcine Serum and Meat Juice from Chickens Using Fluorescent Beads

Microorganisms include a wide variety of bacteria, moulds (fungi), parasites and viruses. Pathogenic micro-organisms have attracted much attention from the public as consumers of contaminated food and water, which resulted in family or community outbreaks. As a consequence, the media and politicians have played their part in increasing consumer awareness and new legislation is in preparation or already in force.

With respect to pathogenic micro-organisms, special attention is drawn to a number of zoonotic diseases, i.e. microbes transmissible from animals to human, for the following reasons: 1) most food- and waterborne diseases in human are zoonotic by nature; 2) many zoonotic agents have their transmission route through the environment, and 3) both contamination of food/water and environment are also used by (bio)terrorists to acquire maximum impact in the society.

Microbiological hazards can enter food chains at any point during pre-harvest, production, processing, transport, retailing, domestic storage or meal preparation. From their introduction on feed or food, highly complex environments can occur in which the micro-organism can elude detection and inactivation. Efficient international distribution systems and rapid changes in consumer preferences can facilitate the swift penetration of pathogens through large populations, greatly shortening the reaction time available to public health agencies.

Authorities and food producers are convinced that rapid fast, versatile and selective (diagnostic) assays are needed for environmental, feed and food monitoring to react adequately to contaminated links in the food chain. A large portion of the explored monitoring techniques involved the use of affinity assay technologies, including biosensor platform.

In principle, detection of the presence of micro-organisms can be carried out in two ways: directly or indirectly. In the direct assay, the organism itself is detected usually with the application of antibodies reacting with (sub)species- and/or strain-specific antigenic structures. This immunochemical analysis follows time-consuming sample preparation through culturing in selective growth media. In the case of parasite infections, this is not possible and direct detection involves microscopic inspection of samples. In the indirect assay, the presence of the micro-organism is suggested by the detection of humoral (immunoglobulins) or cellular (e.g. cytokines) products of an immunological response of the infected host. In most studies, well-defined antigens are used to capture host's immunoglobulins in any body fluid (serology). The observed binding then reveals the nature of an invasive infestation of a pathogen. The advantages and disadvantages of indirect and direct pathogen detection are clear: i) individuals are not always immunologically responding to an infection; i.e. differences between low or high immune responders, ii) humoral responses are delayed several days or even weeks possibly leaving a recent infection unnoticed, iii) serum antibodies can be found where the causative organism is not detected, as it has been rejected or retracted itself in certain (non-sampled) tissues, iv) serological investigations are very fast and offer better possibilities for high-throughput than direct detection, and v) serologic analysis of serum or plasma predicts the Salmonella infection status of a flock or herd better than direct antigen analysis, i.e. classical selective bacterial culturing.

In fact, serology outperforms direct, and in most cases insensitive detection of tissue parasites, which can only be carried out by histochemistry or digestion techniques and microscopy. Significant differences are also apparent in sample collection and preparation: whereas bacteria, fungi and viruses have to be cultured from matrices to facilitate their detection in enriched solutions, blood is relatively easily collected and prepared for analysis. Here, it should be noted, however, that antibodies can not only be retrieved from blood, plasma or serum, but also from muscle (meat juice), milk, colostrums, cerebrospinal fluid and eggs. In particular, sampling of eggs, meat juice and/or milk is easier and more cost-effective than the sampling of blood, plasma, serum or cerebrospinal fluid.

Diagnostic methods based on serologic analysis of antibody-containing biological materials are therefore supportive in so-called logistic slaughtering of animals. In this innovative processing approach, evidence-based and reliable decisions are made on the basis of continuous and intensive monitoring on farm level whether animals are allowed to enter a Salmonella-free or a Salmonella-contaminated processing infrastructure.

Among the components of the antigenic structure of the genus Salmonella, the somatic antigens are important as an instrument to trace immune response in animals upon an invasive infection of this organism. Somatic antigens are located on the polysaccharide part of lipid polysaccharide (LPS), which is a constituent of the bacterial cell wall. Detection of a humoral response with carefully chosen LPS, the identity of the serogroup of the infecting Salmonella can be deduced.

In Denmark, Germany, Greece and The Netherlands, 39.5% of all Salmonella-positive pigs sampled at the abattoir were determined as S. typhimurium. Dependent of country, other important isolates from pigs were S. derby (17.1%), S. infantis (8.0%), S. panama (5.1%), S. ohio (4.9%), S. london (4.4%), S. livingstone (3.1%), S. virchow (2.7%), S. bredeny (2.1%), S. mbandaka (1.1%), S. Brandenburg (1.0%), S. goldcoast (0.8%).

In case of chickens, 14% of the chickens were Salmonella-positive at flock level in 2002 in The Netherlands. The predominant serovar was in that case S. paratyphi B var. java. The retail level a comparable percentage (13.4%) was found in the Netherlands. The most frequent Salmonella serovars isolated from broilers in 14 EU member states were S. paratyphi B var. java (24.7%), S. enteritidis (13.6%), S. infantis (8.0%), S. virchow (6.7%), S. Livingstone (5.7%), S. mbandaka (5.5%), S. typhimurium (5.3%), S. senftenberg (5.0%), S. hadar (3.7%). S. paratyphi B var. java is dominating, but this is fully attributable to The Netherlands.

In food-producing chickens and swine, the prevalently occurring Salmonella serogroups are thus belonging to groups B, C and D, and in the case of swine also E.

In this study, a new analytical affinity assay platform is explored for the indirect detection of Salmonella infection in pigs and chickens. This technology platform from Luminex analyses internally coded beads which can be coated with different antigens in a single test. Only when both fluorescence of bead and bound analyte pass the detector a response will be recorded. This approach is applied to detect anti-Salmonella antibodies in serum and meat drip.

### Materials and Methods

### Chemicals

Amine coupling kit, consisting of N-hydroxysuccinimide (NHS), 1-ethyl-3-(3-dimethlylaminopropyl)carbodiimide hydrochloride (EDC) and ethanolamine hydrochloride-sodium hydroxide pH 8.5 were bought from Biacore AB (Uppsala, Sweden). Ethanol and trichloroacetic acid (TCA) were purchased from Merck (Darmstadt, Germany). Sodium cyanoborohydride and carbohydrazide were obtained from Fluka Chemie GmbH (Buchs, Switzerland). Porcine hemoglobin (Hb) was acquired from Sigma Chemical Company (St. Louis, Mo., U.S.A.). Water was obtained from of a Milli Q water purification system (Millipore, Bedford, Mass., U.S.A.).

### Materials

NAP-5 columns (0.5 ml; Sephadex G-25) were purchased from Amersham Biosciences and were used as described by the producer. CM5 biosensor chips were bought from Biacore AB. Dialysis bag (Spectra/Por) with a cut-off of 1 kDa was obtained from Spectrum Laboratories Inc. (Rancho Dominguez, Calif., U.S.A.). Alexa532 was from Molecular Probes (Leiden, The Netherlands). Goat anti-swine IgG (H+L) was ordered from Jackson Immunoresearch (West Grove, Pa., USA). This antibody was conjugated with Alexa532 using standard labelling procedures.

### Anti-Salmonella Antisera

Salmonella monovalent ‘O’ somatic monoclonal antisera against O4, O5, O6₁, O7, O8, O9, O10 were purchased from Sifin (Berlin, Germany). Antibody solutions were diluted in 50 mM PBS to their working concentrations.

### Reference Avian and Porcine Sera

See example 1, section 1.1.4.

### Methods

### Extraction of LPS

See example 1, section 1.2.1.

### Oxidation of LPS

See example 1, section 1.2.3.

### Immobilization of LPS

To immobilize the oxidized LPS antigens to the beads, the carboxylic groups at the bead surface were activated with a mixture of EDC/NHS available from the amine-coupling kit for 20 min on a gyro rocker. Following centrifugation and removal of supernatant, activation was followed by a reaction with 5 mM aqueous carbohydrazide for 20 min. Beads with modified surface were pelleted again and upper liquid was discarded before addition of 1 M ethanolamine and incubation for 20 min. Following another centrifugation step at 14,000 g for 5 min, oxidized LPS solved in sodium acetate pH 4.0 was added to allow immobilization for 90 min. Following removal of the suparenatant acquired through centrifugation, the linkage between bead-surface and antigen was stabilized using 100 mM sodium cyanoborohydride solved in 10 mM sodium acetate at pH 4.

### BioPlex Assay

Following the warming-up of the bead counter device, this BioPlex (BioRad, Veenendaal, The Netherlands) was calibrated according to the instructions of the producer using a BioPlex calibration kit (BioRad). Samples were diluted in 50 mM PBS in wells of a microtiter plate which were then supplemented with 50 μL 5000 beads/mL LPS-coated beads. The antigen-antibody binding was allowed for 30 min on a microtiter plate shaker operated at 200 rpm. Then 10 μL goat anti-swine IgG (H+L) tagged with Alexa532 fluorescent labels diluted 8 times in 50 mM PBS were added and incubation was continued for 15 min on the shaker. Beads were then analysed for their fluorescence profiles for 30 s on the BioPlex machine.

### Results and Discussion

Fluorescent beads were prepared for coating with LPS from different specific Salmonella serovar sources representing serogroups B, C and D relevant as zoonoses in foods from chicken and swine. It should be noted that serogroup E, which is relevant for pork products, is not studied here. Following the immobilization of each type of LPS to individual beads, which are internally coded, the success of the coating was assessed using commercially available monoclonal antisera against somatic antigens O4, O5, O7, O8 and O9. However, while anti-O5 gave a response of 6398 units, anti-O9 gave 145 units, whereas the background signal of non-matching antigens-antibodies was less than 91 units in all cases (FIG. 6). In a similar way, anti-O4 and anti-O7 gave responses of 305 units and 174 units, respectively (FIG. 7). These differences in responses between commercially available antisera preparations were in very good correspondence with those observed using a surface Plasmon resonance (SPR) biosensor and reflect differences in antibody titers.

In a similar way, the activity of identical LPS batches oxidized on different days were tested using a similar panel of commercial antisera (FIG. 8). Compared to the other oxidation batch, responses ranged between 57% and 148%, which is susceptible for improvements.

Different preparations of meat drip, i.e. juice that is acquired from muscle tissue following a freeze and thaw cycle, and sera from chickens were analysed (FIG. 9). Recorded activities were as expected. Meat drip, serum and a mixture of meat drip and serum from Salmonella-free chickens gave low abundant fluorescent conjugated beads. In contrast, anti-S. pullorum and anti-S. gallinarum should give a response on serogroups B and D, as it contains antigens O1 and O12, which it does for drip and serum. S. infantis contains antigen O6, which is shared by C₁ and C₂. Indeed, this activity is observed in drip and serum.

Besides chicken serum, prepared swine serum were tested as well (FIG. 10). Serum from Salmonella-free pigs gave MFI responses in the range of 110 units (serogroup C₂) to 137 units (serogroup B) and were close to the responses of beads only incubated with buffer, namely from 94 units (serogroup D) to 129 units (serogroup C₂). As expected, significant signals were recorded when sera were spiked with anti-S. typhimurium and S. livingstone antisera, namely 969 units on serogroup B and 207 units on serogroup C₁, respectively. The spiked sera did not react with non-corresponding antigens giving responses between 104 MFI units and 131 MFI units.

### Example 4A

Detection of Anti-Salmonella Antibodies in Poultry Serum and Meat Juice Using ImmuSpeed™

As described, analysis of antibodies in body-derived biological materials, including blood, drip, sera, plasma, milk, etc., can be performed using various technologies to determine Salmonella infections in pigs and chickens. Here, a platform is applied, which is developed by DiagnoSwiss (Monthey, Switzerland) and exists of a disposable chip on which a reservoir, microfluidic system and microelectrodes for detection purpose are combined.

A single chip consists of eight parallel channels. A single channel contains an area with integrated electrodes. It is in this area of each channel in the chip that can be spotted with a varying number of antigens. Here, serogroup representing B, C₁, C₂, D and E are immobilised in such a way that the chip can be reused for repeating analyses on the same chip. In this way, eight samples can be analysed simultaneously every 8 min or faster.

The detection is based on the generation of an electroactive product, which is monitored by the integrated microelectrodes. For that purpose, following incubation of biological samples on the chip, a secondary antibody is introduced on the chip. This secondary antibody is labelled with enzymes for example β-galactosidase, horse-radish peroxidise (HRP) or (alkaline) phosphatase. Electro-inactive substrates are converted when this enzyme activity is present and will induce an electrochemical reaction, which is recorded, when a suitable potential is applied.

### Materials and Methods

### Chemicals and Materials

Water was obtained from of a Milli Q water purification system (Millipore, Bedford, Mass., U.S.A.). p-Aminophenyl-phosphate (C₆H₆NO₄PNa₂.H2O) was from Universal Sensors Inc. (Kinsale-Sandycove; Ireland). Goat anti-swine IgG (H+L) and Donkey-anti-chicken IgG (H+L) was ordered from Jackson Immunoresearch (West Grove, Pa., USA). These antibodies were conjugated with alkaline phosphatase. ‘Solution B’ was ordered from Agilent (Santa Clara, Calif., USA). Oxidized and protein-fortified Salmonella LPS representing serogroups B, C₁, C₂ and D were acquired from experiments described above.

### Reference Avian and Porcine Samples

See example 1, section 1.1.4.

Meat drip was derived from muscle tissue which was originated from an experiment in which two chickens (animal numbers 1236 and 1429) were experimentally challenged with Salmonella enteritidis. Negative samples were from control chickens that were not infected. Muscle tissue was frozen and thawed and the remaining liquid was collected as a meat drip sample.

### Methods

### Immobilization of LPS

Oxidized protein-LPS antigens (0.5 mg/mL) was diluted 1:4 (v/v) in PBS at pH 4. The LPS was coated on an ImmuSpeed™ chip (labelled Loop9-Var1; DiagnoSwiss, Monthey, Switzerland) configured with eight channels. Here, a single channel was coated with a single LPS antigen. Coating was accomplished as follows. In order to activate the sensor channels, the chip was pre-wetted with ethanol, which was contained by the chip's reservoirs, at a flow rate of 10 μL/min. The reservoir was emptied, filled with 30 μl phosphate buffer at pH 4, and channels were flushed with this solution. Each reservoir was emptied and then filled with a solution of oxidized Salmonella LPS-protein complex for coating. The flow rate was set at 10 μl/min and coating was accomplished by applying so-called cycles, i.e. following each 2 s the flow was stopped for 30 sec. This coating procedure was executed for 8 min. After emptying the reservoir, 30 μl blocking agent, consisting of 5% foetal calf serum (FCS) in 0.2 M Tris maleate at pH 6.2, was added and flowed in the same way over the surface. Finally, the reservoir and the channel were washed with PBS containing 0.1% (m/v) BSA and 0.05% (m/v) Tween20 at 10 μl/min through 2-s flow and 10-s stop cycles.

### ImmuSpeed™ Assay

Following its warming-up, an ImmuSpeed™ device (DiagnoSwiss, Monthey, Switzerland) was calibrated according to the instructions of the producer. Samples were diluted 1:100 to 1:1000 (v/v) in 10 mM Tris/HCl pH 7 as indicated in the text. A volume of 30 μL diluted sample was pipetted in an empty chip's reservoir. The sample was flowed though the channel by setting the flow rate at 10 μL/min using 5 cycles each consisting of a flow for 2 s and an arrest for 15 s for an optimal immunoreaction. Reservoirs were emptied, and reservoirs and channels were washed with PBS containing 0.1% (m/v) BSA and 0.05% (m/v) Tween20 at a flow rate of 10 μL/min using 5 cycles. A single cycle consisted of a flow for 2 s and then no flow for 15 s. Then; the reservoir was emptied and filled with 30 μL either goat anti-swine IgG (H+L) or 30 μL donkey anti-chicken IgG (H+L) tagged with alkaline phoshatase, depending of the targeted analytes. These antibodies were diluted 1:150 to 1:1500 (v/v), as indicated, in Tris 100 mM at pH 7 fortified with 1% (v/v) FCS. The solution was introduced into the chip at a flow rate of 10 μL/min and incubation was allowed for 4.25 min through 15 cycles with intermittent pauses of 15 s following 2 s of flowing. Following washing of reservoir and channel as described above, 2 mM PAPP was introduced at 10 μL/min using 2 cycles of a flow for 10 s and a stop of the flow for 15 s, to initiate the generation of an electroactive product. This product was monitored by the integrated electrodes, which were set at 250 mV to give an electrochemical reaction, and by setting 30 cycles at 10 μL/min.

To facilitate a new series of repetitive analysis, the surface was regenerated using the so-called Solution B in 5 cycles (2 s flow at 10 μL/min, 10 s flow stopped) followed by a washing step using PBS containing 0.1% (m/v) BSA and 0.05% (m/v) Tween-20 in 5 cycles (2 s flow at 10 μL/min, 10 s flow stopped).

### Results and Discussion

In a first setting, an ImmuSpeed™ chip was coated with LPS serogroup D on channels 1 to 4, while channels 5 to 8 remained unchanged. The LPS D-positive meat drip that was collected from chickens, which were challenged with Salmonella enteritidis, was contacted with channels 1, 2, 5 and 6 (FIG. 44). The generation of an electroactive product was followed in the time and was an indicator of the presence of anti-salmonella antibodies in the chicken meat drip. As expected, a steep response was developed in channels 1 and 2 in which meat drip reactive with Salmonella serogroup D LPS was injected, while the Salmonella negative samples and the channels, which were not coated with LPS, did only develop a low background response. Repetitive analysis using the same chip following regeneration showed a high degree of comparable results.

A new chip was prepared by coating two series of LPS serogroup B, C₁, D on channels 1, 2 and 3, respectively, and on channels 5, 6 and 7, respectively. Channels 4 and 8 remained uncoated. In a similar way as described above, chicken and porcine sera were analysed revealing a discriminative ability of the assay, as sera were correctly recognized as Salmonella-positive and Salmonella-negative samples. As expected, serogroup D positive samples gave a minor reaction on the LPS B channel. Also in this case, results from repetitive analyses showed a low degree of variance.

### Example 4B

### Detection of Anti-Salmonella Antibodies in Poultry Serum and Meat Juice Using an Interferometry-Based Octet™ Biosensor

Another example of a biosensor technology is given, which can be used to analyse antibodies in various biological materials, which were expressed in these matrices as a result of a humoral response following an exposure of the organism towards Salmonella spp. In this case the platform of interest is the Octet™ produced by FortéBio (Menlo Park, Calif., USA). The detection system of this instrument is based on optical interferometry and measures the phase change of electromagnetic radiation (light) when sensing waves interact with the biolayer surface to which anti-salmonella antibodies can be bound provided that the sensor surface was loaded with Salmonella-specific LPS antigens. Before the density of the biolayer is thus assessed, the disposable single-use biosensors have to be configured with LPS serogroup antigens B, C₁, C₂, D and E and incubated with samples.

### Materials and Methods

### Chemicals and Materials

Water was obtained from of a Milli Q water purification system (Millipore, Bedford, Mass., U.S.A.). Amine coupling kit, consisting of N-hydroxysuccinimide (NHS), 1-ethyl-3-(3-dimethlylaminopropyl)carbodiimide hydrochloride (EDC) and ethanolamine hydrochloride-sodium hydroxide pH 8.5, was bought from Biacore AB (Uppsala, Sweden). Sodium cyanoborohydride and carbohydrazide were obtained from Fluka Chemie GmbH (Bucks, Switzerland). Oxidized and protein-modified Salmonella LPS representing serogroups B, C₁, C₂ and D were acquired from experiments described above. Disposable amine reactive biosensor devices, at which surface carboxylic groups are expected, were obtained from FortéBio.

### Reference Sera

See example 1, section 1.1.4. These sera were diluted 1:500 (v/v) in PBS pH 7 prior to the Octet™ biosensor analysis. Monoclonal antiserum against Salmonella O5 antigen (serogroup B) was purchased from Sifin (Berlin, Germany).

### Methods

Immobilization of LPS and Octet™ Biosensor Analysis

Octet™ samples are presented in a standard 96-well microtitre plate for immobilisation reactions and sample analysis. For immobilisation of the antigens external of the detection device, a microtitre plate was prepared by filling the eight-well rows each with the following solutions: row 1) PBS pH 7, row 2) EDC/NHS, row 3) carbohydrazide, row 4) ethanolamine hydrochloride-sodium hydroxide pH 8.5, row 5) PBS pH 7, row 6) Oxidized protein-LPS antigens (0.5 mg/mL), row 7) cyanoborohydride, row 8) PBS pH 7, row 9) samples, row 10) 1 M urea containing 0.1% (m/v) of each CHAP, Tween-20, Tween-80 and Triton-100, row 11) PBS pH 7 and row 12) samples. The LPS antigens were pipetted as serogroup B, C₁, C₂ and D in the first four and in the second four wells in row 6. Biosensors were positioned appropriately in an 8×12 configured frame. The following incubations were executed outside the machine on a horizontal orbital shaker at 45 rpm: wetting off the biosensors in PBS (row 1) for 2 min, activation of the surface with EDC/NHS (row 2) for 5 min and carbohydrazide (row 3) for 10 min, followed by blocking of remaining active groups (row 4) for 10 min, washing with PBS (row 5), immobilisation of the antigens (row 6) for 90 min and stabilisation of formed bonds using cyanoborohydride (row 7) for 90 min and finally a wash step with PBS (row 8) for 5 min.

In the instrument the following steps were automatically executed, in which case solutions were homogenized by shaking the microtitre plate while the incubation occurred. The biosensors were submerged in a PBS solution for 3 min before contacting the samples (row 9) for 5 min. Dependent of the experiment, biosensors were regenerated using the solution in row 10 for 1 min and prepared for a next analysis by dipping the sensors in PBS (row 11) for 3 min. Samples (row 12) were then again contacted for 5 min before detection as described. Regeneration and analysis of samples was repeated thrice.

### Results and Discussion

As biosensor conditions have to be optimized for this specific Salmonella test, as in a research and development phase of any assay, several steps to prepare the biosensors for sample analysis were performed manually outside the machine. Apparently, these conditions, including the immobilisation of LPS antigens, were satisfactory as specific responses were obtained after the analysis of biosensors that were contacted with reference sera.

As an example, the analysis of chicken serum positive for Salmonella pullorum-galinarum (serogroup D₁) and of negative chicken SPF serum is given in FIG. 45. The biosensor used in this case was immobilised with serogroup B LPS and although S. pullorum-galinarum belongs to serogroup D, it contains antigen O12, which is also found in serogroup B. The observed signal was therefore expected. The analysis also showed a comparable three repetitive analysis on a single biosensor with intermittent regeneration.

New serogroup B LPS antigen-active biosensors were prepared to assay a monoclonal anti-O5 antibody (FIG. 46). As expected, this antibody reacted with the immobilized LPS, while negative sera did not give or even gave a negative response. Remarkably different intensities of the signals at the different biosensors were obtained while probing the identical sample.

In conclusion, biolayer interferometric analysis of serum antibodies using biosensor-immobilised protein-LPS complexes was successful to distinguish Salmonella-positive and Salmonella-negative samples. As expected, serogroup D positive samples gave a minor reaction on the LPS B biosensor.

### Example 5

### Determination of Anti-Salmonella Antibodies in Exotic Avian Species Using an SPR Biosensor

### Aim of Study

The aim of this investigation was to explore whether the developed SPR biosensor technology based on the use of immobilized selected Salmonella LPS to detect indirectly Salmonella infections in food-producing animals, is able to detect such infections in exotic animal species as well.

### Materials

Plasma from tocotoucans (Rhamphastos toco) and a sharp-tailed grouse (Tympanuchus phasianellus), which were infected with S. typhimurium of different phage types, were kindly provided by Dr. W. Schaftenaar and Ing. M. de Boer (Veterinary Department, Rotterdam Zoo, The Netherlands). The disease history of these animals is the following.

From the faeces of a Tocotoucan sampled at Mar. 24, 1994, S. typhimurium phagetype 292 was isolated. From another faeces sample of the same bird, S. typhimurium phagetype 352 was isolated at Jun. 28, 1994. Aug. 24, 1994, plasma was collected from this animal used for SPR analysis in this study.

Blood was collected from a diseased sharp-tailed grouse at Oct. 28, 1997. The plasma prepared from this blood was used for analysis in this study. This animal died the next day. S. typhimurium phagetype 507 was isolated from the dead bird.

### Methods

An SPR biosensor (Biacore 3000) containing a sensor chip of which flow channels were coated with LPS from S. enteritidis, S. livingstone, S. goldcoast and S. typhimurium, was operated as described earlier. Plasma samples were diluted as described for sera in examples 1 and 2 and analysed.

### Results and Discussion

The invention was in the first place developed for application in the food chains to secure the safety of food of animal origin with respect to Salmonella contaminations. Nonetheless, the field of applicability was tested with plasma collected from two exotic avian species, namely a tocotoucan (R. toco) and a sharp-tailed grouse (T. phasianellus), which were infected with S. typhimurium as disclosed by classic microbiological diagnostics (personal communication with M. de Boer, Blijdorp Zoo, Rotterdam).

The faeces of the tocotoucan was found positive five and two months before blood was sampled and a humoral response could develop over a relatively long period of time. Indeed, the biosensor response was high (Table 33). Compared to blank serum from SPF chickens and to standard antiserum (anti-serogroups A to S), the reactivity with S. typhimurium LPS was dramatic high (4185 response units (RU)). A response was also observed on the channel of S. enteritidis (1220 RU), which was also observed for serum from chickens highly infected with exclusively. S. typhimurium and is in accordance with the presence of somatic antigen O12 in both serovars and thus in serogroups B and D (cf. Tables 1 and 3). Unexpectedly, a relatively high response was also observed at the S. goldcoast channel. It can not be excluded here that this bird was infected with multiple Salmonella serovars simultaneously or sequentially with S. typhimurium as the last infection, including a C₂ infection.

The plasma of the sharp-tailed grouse was not very reactive with the different LPS types, but reactivity was in all cases higher than that of the blank serum and on Sl and St LPS higher than that of the reference antiserum (Table 33). As the bird died rapidly from the infection, a significant humoral response against the selected antigens was probably not fully developed and not detected by the biosensor. It should be noted that serology is therefore not very suitable for diagnosis on an individual level. As evidenced by Swanenburg (Utrecht Thesis, Utrecht University, 2000), serology is, in particular, suitable for assessing the Salmonella status on a population level.

TABLE 33
Results of the analysis of plasma collected from a tocotoucan and a
sharp-tailed grouse which were infected with S. typhimurium.
The results are expressed in relative response units. Samples
were analysed thrice.

| Flow | Immo- | | | | Plasma |
|---|---|---|---|---|---|
| channel | bilisation | | | | from |
| coated | level | Serum | Anti- | Plasma | sharp- |
| with LPS | of probing | from SPF^{a} | serogroup | from | tailed |
| from | LPS | chickens | B^{b} | tocotoucan | grouse |
| Se | 3241 | 17 (3) | −3 (1) | 1220 (304) | 33 (1) |
| Sg | 3354 | 9 (4) | −8 (1) | 492 (186) | 59 (9) |
| Sl | 5241 | 6 (6) | −29 (2) | 58 (12) | 89 (8) |
| St | 2755 | 46 (6) | 663 (33) | 4185 (362) | 73 (8) |

^{a}serum from specific pathogen free chickens, i.e. blank serum
^{b}commercially available monoclonal antibody reacting with Salmonella serogroup B.

### Example 6

Detection of Bacteriophage Felix O1 (Fo1) Through its Binding to Salmonella LPS Immobilized on an SPR Biosensor Chip Surface

### Goal

To determine the binding of the bacteriophage FO1 to LPS immobilized to the biosensor surface

### Approach

To prove binding of bacteriophage Felix O1 (FO1, Félix d'Hérelle Reference Centre for Bacterial Viruses, Laval, Canada) to Salmonella LPS, the bacteriophage was diluted in HBSEP to obtain a concentration series. These samples were injected for 2 min on the biosensor to allow binding to LPS from S. typhimurium, S. enteritidis, S. goldcoast and S. livingstone, which were each immobilized separately on an individual flow channel of the sensorchip.

### Results

The results are summarised in FIG. 11.

Although a relatively high concentration of bacteriophage was needed to obtain a significant response, it is evident from this experiment that 10⁹ PFU of FO1 bacteriophages/mL and higher bound to LPS coupled to the chip surface.

### Example 7

### Detection Bacteriophage FO1 Through its Binding to S. typhimurium LPS Immobilized on an SPR Biosensor Following its Incubation with S. typhimurium, S. enteritidis, S. goldcoast and S. livingstone

### Goal

To Determine the Binding of Bacteriophage FO1 to a Live Culture of Salmonella spp. in HBSEP.

### Approach

Different Concentrations of Cultures of S. typhimurium, S. enteritidis, S. goldocoast, S. livingstone and blank medium were mixed for 5 min with 1.2×10⁹ PFU bacteriophage FO1. After incubation, bacteria were spun down and supernatant was analysed on the Biacore with a biosensor chip containing immobilized LPS from S. typhimurium.

### Results

In order to determine significant responses, a cut off value was established by the averaged readings of blank medium containing no Salmonella but 1.2×10⁹ PFU bacteriophage, minus 3 times standard deviation. Applying this value disclosed that Salmonella should be present at a concentration of at least 6×10⁸ CFU/mL, 3×10⁶ CFU/mL, 4×10⁷ CFU/mL and 3×10⁴ CFU/ml for S. typhimurium, S. enteritidis, S. goldocoast, S. livingstone, respectively, to give a significant response (FIG. 12).

### Discussion

The absorption rate of bacteriophage FO1 to Salmonella spp. is probably dependent of the accessibility of N-acetylglucosamine in the core region, the binding site of FO1 (Lindberg, 1977; Lindberg and Holme, 1969). Long and numerous O-side chains occurring in the polysaccharide region of the LPS of targeted Salmonellae could, therefore, impair the binding of FO1 to the analyte. It is, therefore, expected that free bacteriophages will have a variable binding capacity towards Salmonella strains and that propagation of the virus will largely depend on the molecular profile of the exposed LPS.

When immobilizing LPS-protein complexes at the surface of a solid carrier for a diagnostic method, as described in the invention, a dense network of ligands may be formed. In the presented Biacore analysis, the density Of the complex and the hindrance by the proteins may play a role in the observed difference in bacteriophage binding to the four LPS types.

It should be noted here that, as discussed in the invention as well, oxidation of monosaccharide constituents in the core region is expected, including that of the N-acetylglucosamine. The ligand for the bacteriophage is, therefore, affected and this may influence the sensitivity of the test.

### Example 8

### Propagation of Bacteriophage FO1 in Salmonella and Non-Salmonella Strains

### Goal

The propagation of FO1 is not exclusive in Salmonella spp., but possibly also in non-Salmonella strains (3). This study was initiated to investigate the selectivity of the of proliferation of the bacteriophage FO1. For this purpose, a number of important food non-Salmonella pathogens were exposed to the FO1 bacteriophage.

### Methods

Seven non-Salmonella strains (Listeria monocytogenes, Escherichia coli, Pseudomonas aeroginosa, Bacillus cereus, Citrobacter, Enterococcus feacalis, and Staphylococcus aureus) and seven Salmonella strains (S. cholerasuis, S. berta, S. meleagridis, S. Agona, S. pullorum, S. Virchow and S. enteritidis) were grown in Tripton Soy Broth (Oxoid CM129).

At t 0 hours, 1.2×10⁸ PFU of FO1 (end concentration 1×10⁶ PFU/ml) was added to all cultures. Every hour a sample was drawn, and absorbance at λ 600 nm (FIGS. 13 and 14), plaque forming units/ml (FIGS. 15 and 16) and, after concentration and buffer exchange, binding to immobilized St-LPS immobilized on a sensorchip surface in a Biacore biosensor (FIG. 17), were determined.

Some non-Salmonella bacteria, including L. monocytogenes, P. aeroginosa, E. faecalis and Staph. aureus, did not show growth in five hours of culture (FIG. 13). These bacteria were obviously not loaded nor infected by the bacteriophage FO1, because the concentration of bacteriophages did not rise over time and was stationary at 1×10⁶ PFU/ml (FIG. 15).

In contrast, all Salmonella serovars, except S. virchow, grew in five hours of incubation (FIG. 14). This S. virchow strain was probably lysed completely by the proliferating bacteriophages, as a clear increase in the concentration of bacteriophages can be shown from 1×10⁶ PFU/ml to 1×10¹⁰ PFU/ml (FIG. 16). In a similar way, S. berta and S. meleagridis bacteria showed an increase of bacteriophages concentration. However, these bacteria, in particular, S. berta showed good growth (FIG. 14).

In the context of the invention, the binding of propagated bacteriophages to the sensor surface is of greatest interest. For this purpose, bacteriophages propagated in Salmonella were concentrated, dialysed and serially diluted before SPR biosensor analysis (FIG. 17). Unexpectedly, comparable bacteriophage concentrations gave dramatic different biosensor responses. Most probably phages were lost, in particular during concentration and dialysis step, during the sample preparation. Nevertheless, the preparations of bacteriophages, which have shown propogation (cf. FIG. 16) gave clearly higher responses than the blank sample, which contained the staring concentration of bacteriophages only.

### Conclusion

These experiments showed that bacteriophages can be used as an analytical tool to detect the presence of Salmonella in samples and that Salmonella LPS immobilized to a solid surface can be used to probe the increment of the bacteriophages as a result of propagation of the virus in the host bacteria following a short incubation period.

### Example 9

Immobilisation of Salmonella-Derived LPS onto Fluorescent Beads and Detection of Antibodies Reporting, a Current or Past Salmonella Infection in Various Biological Samples

### 1. Introduction

Among the components of the antigenic structure of the genus Salmonella, the somatic antigens are important as an instrument to trace immune response in animals upon an invasive infection of this micro-organism. Somatic antigens are located on the polysaccharide part of lipid polysaccharide (LPS), which is a constituent of the bacterial cell wall. After extraction and isolation of LPS from carefully chosen Salmonella serotypes (cf. SOP CHEMIE/A21), antigen-containing LPS is coupled covalently to beads, which are internally coded by a specific mixture of fluorescent material. One of the exploited technology platforms is from Luminex, which can identify up to 100 differently internally coded beads in a single test. A single species of beads can be immobilized with a mixture of LPS reflecting different serogroups, or different species of beads can be each immobilized with LPS reflecting a single specific serogroup or serovar of the pathogenic micro-organism. The LPS-containing beads are incubated with body-derived materials, such as blood, plasma, serum, meat drip/juice, egg-yolk, milk etc, to enable anti-Salmonella antibody-antigen binding. The specific binding is detected following a second incubation with fluorescently tagged anti-immunoglobulin antibodies in a device analyzing simultaneously the emission wavelengths of excitated beads and tagged antibodies. Only when both fluorescence of bead and antibody are detected simultaneously a response to a specific bead species will be recorded.

This SOP describes the method for oxidation, immobilization of LPS onto beads and an assay to assess the quality of produced.

### 2. Scope and Field of Application

To analyze biological fluids, such as serum samples from chicken and pigs, for the presence of anti-Salmonella antibodies reacting with O3, O4, O5, O6, O7, O8, O9, O10 and O12 somatic antigens reflecting a history of or current infection of Salmonella from serogroups B, C, D and E.

### 3. References

Extraction and isolation of lipopolysaccharides from Salmonella spp.; Immobilisation of salmonella-derived LPS onto a biosensor chip (Biacore) and detection of serum antibodies reporting a current or past salmonella infection; Optimalisation of protein addition to LPS for immobilization and detection of serum antibodies; See example 1.

### 4. Definitions

c=concentration in % (m/v), % (v/v), mol/l or mmol/l as indicated.

### 6. Principle

LPS is oxidized in the presence of a protein facilitated by sodium periodate. The LPS-protein solution is desalted using a NAP-5 column. After activation of the carboxylic acid groups at the surface of the beads with the aid of 1-ethyl-3-(3-dimethylaminopropyl) carbodiimide hydrochloride (EDC), N-hydroxysuccinimide (NHS) followed by a reaction with carbohydrazide, desalted oxidized LPS-protein complex is immobilized to the solid phase of the beads. Bound LPS is then stabilized with sodium cyanoborohydride. Prior to routinely use, the performance of bead-conjugated LPS to bind anti-Salmonella antibodies is assessed using a panel of reference monoclonal agglutination sera.

### 7. Reactions

### 7.1 Oxidation of Carbohydrate Moiety

Periodate will induce an oxidative disruption of linkages between vicinal cis-diols on, in particular, carbohydrate moieties, as in e.g. mannose, to yield aldehyde functionalities, see FIG. 18. This reaction is typically performed in buffers at a pH range between 4.5 and 5.5 in the dark using a freshly prepared 10-100 mM sodium meta-periodate in 0.1 M sodium acetate.

NOTE 1: The positions of conjugations indicated in FIG. 18, to link up the depicted monosaccharide with other monosaccharide residues in a polysaccharide, as in e.g. LPS, are here just given as an example. R′ and R indicate the distal and the proximal positions, respectively, in the carbohydrate chain. The oxidation of hydroxyl groups into aldehydes may repeat itself within the polysaccharide chain in each monosaccharide constituent containing susceptible vicinal diols.

NOTE 2: Periodate will also oxidize, when present, certain 8-aminoethanol derivatives such as the hydroxylysine residues in collagen, as well as methionine (to its sulfoxide) and certain thiols (usually to disulfides). In addition, N-terminal serine and threonine residues of peptides and proteins can be selectively oxidized by periodate to aldehyde groups. These reactions, however, usually occur at a slower rate than oxidation of vicinal diols.

### 7.2 Conjugation to Protein

Oxidation is performed in the presence of a protein. The bis-aldehyde compounds, such as the oxidized monosaccharide constituents in the polysaccharide chain of LPS here, may react with any amino group in a protein and may form a Schiff-base linkage resulting in a substituted imine. See FIG. 19.

NOTE: The substituted imine is stabilized while the complex is attached to the bead surface, by a reduction facilitated by cyanoborohydride. This type of reaction scheme is known as a reductive amination.

### 7.3 Immobilization to Fluorescent Beads

The carboxylic acid labeled beads are activated using N-ethyl-N′-(3-dimethyl aminopropyl)-carbodiimide hydrochloride (EDC) and N-hydroxysuccinimide (NHS). The activation is followed by a reaction with carbohydrazine. The reactive aldehyde functionalities react spontaneously with the hydrazide to hydrazones, which are then reduced to stabilise the covalent bonds. See FIG. 20.

NOTE: The protein (R″) in FIG. 20 carries multiple —NH₂ groups and can therefore be conjugated with multiple oxidized LPS entities. At the other hand, the polysaccharide part in LPS may carry multiple free aldehyde groups in a single molecule. These aldehyde groups may for a part or completely captured by the hydrazide-layer on the beads. The net result may be a very stable complex network of protein-LPS covalently linked to the bead surface.

### 8. Reagents and Materials

In the complete procedure only reagents of recognized analytical grade and only distilled water or water of equivalent purity are used, unless stated otherwise. Reference to a company is for information and identification only and does not imply a recommendation unless so stated.

### 8.1 Chemicals

8.1.1 Acetic acid (J.T. Baker, Deventer, The Netherlands)
8.1.2 Amine coupling kit (Biacore AB, Uppsala, Sweden) consisting of
8.1.2.1. Vial containing 115 mg N-hydroxysuccinimide (NHS)
8.1.2.2. Vial containing 750 mg 1-ethyl-3-(3-dimethlylaminopropyl) carbodiimide hydrochloride (EDC)
8.1.2.3. Vial containing 10.5 ml, c=1 mol/l, ethanolamine hydrochloride sodium hydroxide pH 8.5

### 8.1.3. Bio-Plex Calibration Kit (Bio-Rad, Veenendaal, the Netherlands)

### 8.1.4. Carbohydrazide, CN₄H₆O (Fluka Chemie GmbH, Buchs, Switzerland)

8.1.5. Carboxymethyl-dextran sodium salt (Fluka)
8.1.6. Potassium dihydrogen phosphate (KH₂PO₄) (Merck, Darmstadt, Germany)

### 8.1.7. Proclin 150 (Supleco, Bellefonte, Pa., USA)

8:1.8. Monoclonal anti. Salmonella O-antigens:
8.1.8.1. anti-O4 (SIFIN, Berlin Germany)
8.1.8.2. anti-O5 (SIFIN)
8.1.8.3. anti-O6₁ (SIFIN)
8.1.8.4. anti-O7 (SIFIN)
8.1.8.5. anti-O8 (SIFIN)
8.1.8.6. anti-O9 (SIFIN)
8.1.8.7. anti-O10 (SIFIN)
8.1.9. Salmonella LPS, in-house isolated LPS by TCA extraction (SOP CHEMIE/A21) prepared from the Salmonella bacteria serovars enteriditis (Se), goldcoast (Sg), livingstone (Sl), meleagridis (Sm) and typhimurium (St) with protein (SOP CHEMIE/A23)
8.1.10. Sheep anti-mouse Ig-PE (Chemicon, Boronia, Victoria, Australia)
8.1.11. Sodium acetate trihydrate (J.T. Baker, Phillipsburgh, N.J., USA)
8.1.12. Sodium chloride (Merck)
8.1.13. Sodium cyanoborohydride (NaCNBH₃) (Fluka)
8.1.14. di-Sodium hydrogen phosphate (Na₂HPO₄) (Merck)
8.1.15. Sodium hydroxide, c 50 mmol/l (Biacore)
8.1.16. Sodium m-periodate (NaIO₄) (Sigma-Aldrich, Zwijndrecht, the Netherlands)
8.1.17. Water is obtained from a Milli Q water purification system

### 8.2. Solutions

8.2.1. Acetic acid solution, c=0.1 g/ml
8.2.2. Acetate buffer solution, c=10 mmol/l, pH 4.0
8.2.3. Acetate buffer solution, c=1.0 mol/l, pH 5.5 8.2.4. Acetate buffer solution, c=100 mmol/l, pH 5.5 8.2.5. Carbohydrazide solution, c=100 mmol/l
8.2.6. Carbohydrazide solution, c=5 mmol/18.2.7. EDC-solution: reconstitute EDC (8.1.2.2.) in 10.0 ml water.
8.2.7.1. Fractionate 100-μl aliquots of this solution (8.2.7) in polypropylene tube. Store at −18° C. or at lower temperature. The aliquots are stable for two months. Before use: Thaw frozen aliquots and agitate them gently to ensure homogeneous solutions.
8.2.8. Ethanolamine solution: Pipette 200 μl c=1 mol/l ehthanolamine solution (8.1.2.3) in a polypropylene tube
8.2.9. NHS-solution: reconstitute NHS (8.1.2.1) in 10.0 ml water.
8.2.9.1. Fractionate 100-μl aliquots of this solution (8.2.9) in polypropylene tube. Store at −18° C. or at lower temperature.
8.2.10. PBS (5.6), c=100 mmol/l, pH 7.2
8.2.11. PBS, c=10 mmol/l, pH 7.2 8.2.12. Anti-mouse Ig-PE, prediluted: dilute fluorescent conjugate 5 times by mixing 40 μl anti-mouse Ig-PE (0) with 160 μl PBS (8.2.11).
8.2.13. Sodium cyanoborohydride, c=1.00 mol/l
8.2.14. Sodium cyanoborohydride, c=100 mmol/l
8.2.15. Sodium hydroxide, c=5 mmol/l
8.2.16. Sodium m-periodate solution, c=100 mmol/l.
8.2.17. Sodium m-periodate ‘ready to use’: Pipette 100 μl of sodium m-periodate solution, c=100 mmol/l (8.2.16) in a 1.4 ml polypropylene tube and dry with a centrifugal evaporator.
8.2.18. Sodium Periodate Solution, c=50 mmol/l: Dissolve sodium periodate ‘ready to use’ (8.2.17) in 200 μl acetate solution, c=100 mmol/l pH 5.5 (8.2.4). Prepare just before use.

### 8.3. Standard Monoclonal Reference Solution

8.3.1. Concentrated monoclonal Salmonella anti-O5 (8.1.8.2): dilute 5 μl anti-O5 10 times by adding 45 μl PBS c=10 mmol/l, pH 7.2 (8.2.11).
8.3.2. Monoclonal Salmonella anti-O5: 7.5 μl anti-O5 (8.3.1) 10 times diluted by addition of 67.5 μl PBS c=10 mmol/l, pH 7.2 (8.2.11).
8.3.3. Monoclonal anti Salmonella O-antigens (8.1.8): dilute 7.5 μl of each monoclonal (8.1.8.1, 8.1.8.3, 8.1.8.4, 8.1.8.5, 8.1.8.6, 8.1.8.7) with 67.5 μl PBS (8.2.11) in a micro titerplate (9.20).
8.3.4. Thrice diluted monoclonal antibodies: add 25 μl monoclonal antibody solution (8.3.2 and 8.3.3) to 50 μl PBS c=10 mmol/l, pH 7.2 (8.2.11) in wells of the same micro titerplate (8.3.3)
8.3.5. Repeat step 8.3.4 twice to obtain 9 and 27 times diluted antibodies in fresh wells of the micro titerplate in the case of anti-O4, anti-O6, anti-O7, anti-O8, anti-O9 and anti-O10. In the case of anti-O5, these dilution factors were 90 and 270 times, respectively.
8.3.6 Remove 25 μl from the highest dilution (8.3.5).
8.3.7 The antibody solutions are now ready for use.

NOTE The final dilution factors are 100, 300, 900 and 2700 times in the case of anti-O5, whereas in the other cases antibodies were finally diluted 10, 30, 90 and 270 times compared to the original preparation (8.1.8).

### 8.4. Auxiliary Materials

8.4.1 NAP-5 column (0.5 ml, Sephadex G-25, Amersham Biosciences).
8.4.2. COOH-Beads (5.6 μm COOH microspheres) numbers 24, 25, 26, 27 and 28 mixed in 0.01% aqueous merthiolate at 1.25×10⁷ beads/mL (BioRad).

### 10. Software

The BioPlex apparatus is operated with Bio-Plex Manager software 4.1.

### 11. Procedure

### 11.1. Oxidation and Desalting of LPS Solution

### 11.1.1. Oxidation

11.1.1.1. Add 500 μl acetate buffer c=100 mmol/l, pH 5.5 (8.2.4) to dry LPS (8.1.9; See safety precaution)
11.1.1.2. Vortex the solution (11.1.1.1) and sonicate for 20 min and observe the reconstitution process so that all LPS is dissolved.
11.1.1.3. Add 20 μl periodate solution c=50 mmol/l (8.2.18) to the LPS solution (11.1.1.2)
11.1.1.4. Vortex the solution (11.1.1.3)
11.1.1.5. Incubate on ice for 40 min protected from light.
11.1.1.6 Quench oxidation by desalting the solution (11.1.1.4) as described in 11.1.2

### 11.1.2. Desalting

11.1.2.1. Place NAP-5 column(s) (8.4.1) on manifold.
11.1.2.2. Condition the column(s) (11.1.2.1) by passing three 3-ml portions of acetate buffer c=10 mmol/l, pH 4.0 (8.2.2) over the column bed on a flow generated by gravity only. Allow the buffer to enter the gel bed completely.
11.1.2.3. Pipette 0.5 ml oxidized LPS solution (11.1.1.5) on the column. Allow the sample to enter the gel bed completely. The flow-through is not collected.
11.1.2.4. Elute oxidized LPS with 1 ml acetate buffer c=10 mmol/l, pH 4.0 (8.2.2). Collect eluate in a 5-ml glass tube.
11.1.2.5. Vortex (9.21) the solution (11.1.2.4) for 10 s and add 2 μL Proclin 150 (8.1.7).
11.1.2.6. When not immediately used (11.1.2.5) store samples at 4° C. to 7° C.
11.1.2.7. Prior to immobilization, the LPS-containing solution (11.1.2.5) which can be used for different matrix and species applications is diluted as indicated in the following Tables 34 and 35.

TABLE 34
Amount of LPS solution used to immobilize beads for detection of
antibodies to Salmonella O-antigens in swine sera

| LPS type | LPS stock solution | Added volume of acetate |
|---|---|---|
| (8.1.9) | (11.1.2.6) in μl | buffer, pH 4.0 (8.2.2) in μl |
| Se | 75 | 225 |
| Sg | 75 | 225 |
| Sl | 75 | 225 |
| Sm | 75 | 225 |
| St | 75 | 225 |

TABLE 35
Amount of LPS solution used for the immobilization to fluorescent
beads for detection of antibodies in chicken sera reacting
with Salmonella O-antigens.

| LPS type | LPS stock solution | Added volume of acetate |
|---|---|---|
| (8.1.9) | (11.1.2.6) in μl | buffer, pH 4.0 (8.2.2) in μl |
| Se | 150 | 150 |
| Sg | 150 | 150 |
| Sl | 150 | 150 |
| Sm | 150 | 150 |
| St | 150 | 150 |

### 11.2. Immobilization of LPS to Beads

11.2.1. Beads (8.4.2) are vortex-mixed for minimally 1 min
11.2.2. Transfer a portion of 300 μL beads (11.2.1) into a fresh container

### 11.2.3. Centrifuge at 14,000 g for 5 min

11.2.4. Remove supernatant carefully from the beads using a 200 μl pipette
11.2.5. Leave a small amount of solution (10 μL) in the vial and mix the beads in the remaining solution on a vortex.
11.2.6. Thaw two portions of 100 μl EDC (8.2.7.1)
11.2.7. Thaw two portions of 100 μl NHS solution (8.2.9.1).

### 11.2.8 Mix 180 μl of EDC (11.2.6) and 180 μl of NHS (11.2.7).

11.2.9. Transfer 300 μL EDC/NHS mix (11.2.8) to the beads (11.2.4) an suspend rigorously using a pipette.
11.2.10. Facilitate reaction on a gyro rocker for 20 min.

### 11.2.11. Centrifuge at 14,000 g for 5 min.

11.2.12. Carefully remove supernatant from beads, leave a small volume (approx. 10 μL) on top of pellet and suspend beads using vortex mixer.
11.2.13. Add 300 μL 5 mM carbohydrazide solution (8.2.6) to the beads (11.2.12) and suspend rigorously using a pipette.
11.2.14. Facilitate reaction on a gyro rocker for 20 min.
11.2.15. Centrifuge at 14,000 g for 5 min, remove supernatant from beads, leave a small volume (ca. 10 μL) on top of pellet and suspend beads using vortex mixer.
11.2.16. Add 300 μL 1 M ethanolamine solution (8.2.8) to the beads (11.2.15) and suspend rigorously using a pipette.
11.2.17. Facilitate reaction on a gyro rocker for 20 min.
11.2.18. Centrifuge at 14,000 g for 5 min, remove supernatant from beads, leave a small volume (ca. 10 μL) on top of pellet and suspend beads using vortex mixer.
11.2.19. Add 300 μL diluted oxidized LPS in sodium acetate c=10 mmol/l, pH 4.0 (11.1.2.7) and suspend rigorously using a pipette.
11.2.20. Allow reaction on a gyro rocker for 90 min.
11.2.21. Centrifuge at 14,000 g for 5 min, remove supernatant from beads, leave a small volume (ca. 10 μL) on top of pellet and suspend beads using vortex mixer.
11.2.22. Add 300 μL cyanoborohydride solution c=100 mmol/l (8.2.14) and suspend rigorously using a pipette.
11.2.23. Facilitate reaction on a gyro rocker for 60 min.
11.2.24. Centrifuge at 14,000 g for 5 Min and carefully remove supernatant from beads, leave a small volume (ca. 10 μL) on top of pellet and suspend beads using vortex mixer.

### 11.2:25. Add 300 μL PBS (8.2.11).

11.2.26. Add 1 μL Proclin 150 (8.1.7) and mix suspension
11.2.27 The beads are ready for testing Salmonella antibodies in biological materials
11.2.28. Counting of LPS coupled beads
11.2.28.1. Vortex LPS coupled beads suspensions (11.2.25) and transfer 1 μl, in a fresh 1-mL tube
11.2.28.2. Dilute by adding 24 μL PBS c=10 mmol/l, pH 7.2 (8.2.11) and mix.
11.2.28.3 The external supports of a Barker-Turk counting chamber are to be moistened with milliQ water (8.1.17) and the cover glass is gently pushed onto the counting chamber from the front.
11.2.28.4 Fill a pipette with 20 μl bead solution (11.2.28.2), gently form a drop at the tip of the pipette.
11.2.28.5 This drop (11.2.28.4) is to be placed between the cover glass and the counting chamber.
11.2.28.6 As a result of the capillary effect the gap between the cover glass and the chamber base fills up. Before the bead solution can overflow at the edges of the chamber section, the tip of the pipette must be removed. If any air bubbles are visible or if the liquid has overflowed over the edges and into the grooves, the chamber must be cleaned and feeding must be repeated.
11.2.28.7 Place the filled counting chamber under a microscope and magnify the image with a 10× object.
11.2.28.8 The count should be started at the top left-hand corner and follow the direction shown by the arrow (FIG. 23, lower panel). Counting may be enhanced with the microscopes illumination reduced.
11.2.28.9 Count the number of beads in 16 squares (FIG. 23, upper panel) inside the thick lined area (see FIG. 24).
11.2.28.10 Notes on counting:
11.2.28.10.1 Use reduced microscope illumination for all chambers.
11.2.28.10.2 The difference of the counter cells in the large squares and the group squares must not exceed 10 cells.
11.2.28.10.3 Double checks must be performed for all cell counts. After counting the two counting nets the bottom counting net is to be counted in the same way as a check. When doing this it is to be ensured that the chamber has not dried out. This can be prevented by filling the bottom chamber only shortly before the count and the counting after the sedimentation time.
11.2.28.10.4 The difference between the totals of the counts for the two counting nets must not exceed 10 cells. The average value of the counts is then used in the calculation formula or multiplied by the corresponding factor.
11.2.28.11 Multiply the counted number (11.2.28.9) with the dilution factor (25×) divided by counted area (1 mm²) multiplied with chamber depth to calculate the concentration of beads per ml.

### 11.3. Detection of Anti-Salmonella Antibodies

11.3.1. Make the BioPlex apparatus operational by a 30 minutes laser warming up step, followed by a start up and calibration procedure with appropriate calibration solvents (8.1.3) according to the quick guide.
11.3.2. Mix and dilute LPS coated beads (11.2.26) with PBS c=10 mmol/l, pH 7.2 (8.2.11) so that the concentration of each bead equals 5000 per ml.
11.3.3. Transfer 50 μL bead mix (11.3.2) into a micotiter plate already filled with diluted monoclonal anti-O antigens (8.3.7).
11.3.4. Incubate for 30 min on a microliter plate shaker.
11.3.5. Add 10 μL 5 times diluted anti-mouse. Ig-PE (8.2.12).
11.3.6. Incubate for 15 min on a microtiter plate shaker.
11.3.7. Note in logging sample wells and their contents.
11.3.8. Place plate in BioPlex, set maximal counting time to 120 s and count at least 50 beads per LPS group.
11.3.9. Activate software program to count fluorescence of beads, which had captured (fluorescent) antibodies.

For a schematic representation of the procedure see FIG. 21.

Typical responses of salmonella monoclonal antibodies are presented in Table 36.

TABLE 36
Typical responses of reference sera incubated with LPS-coated
beads and a secondary fluorescent antibody providing the signal.

| | LPS B | LPS C₁ | LPS C₂ | LPS D |
|---|---|---|---|---|
| anti-O4 | 305 | 76 | 95 | 88 |
| anti-O5 | 6398 | 78 | 91 | 79 |
| anti-O7 | 86 | 174 | 91 | 98 |
| anti-O8 | 90 | 86 | 1668 | 91 |
| anti-O9 | 82 | 81 | 82 | 145 |

### Example 10

### Mild Periodate Oxidation

The success of the final binding of anti-Salmonella antibodies, and thus the screening of invasive infections in the animal, is much dependent of the oxidation of the monosaccharide constituents of the polysaccharide part of LPS, and the oligossacharide part of the core region of LPS. It can be deduced from the described structures for the different serotypes of e.g. Salmonella that oxidation may lead to a breakdown of the antigenic structures, which are, in particular, part of the polysaccharide part of LPS.

Whereas the oxidation of hexitols occurs rapidly, parynosides, which are predominantly occurring in Salmonella LPS, need a higher periodate concentration to facilitate the oxidation in the same time. Pyranosides, which possess α-erythro-hydroxyl groups, such as in arabin, galacto or manno configurations like in Salmonella spp. LPS, are easier oxidized than α-threo-hydroxyl groups, such as in xylo or gluco variants. It should be realized that while the ring is opened and aldehyde functions for attachment of e.g. protein molecules are created, also α-hydroxy carbonyl compounds may be created, which may oxidize again if periodate is still present. It is, therefore, that non conjugated monosaccharides, which are thus not part of an oligo- or polysaccharide, are completely destroyed by a periodate oxidation to formic acid and formaldehyde at sufficient high concentrations of the oxidizer. At relatively high concentrations of periodate, 1,3-diketones and also di-axial diols can be oxidized.

A breakdown or oxidation reaction more than only the creation of aldehyde groups would, therefore, lead to a failure to detect an infection in a sample of biological material despite a good coupling reaction to a solid phase supported by the presence of a polyamine-containing molecule, such as a protein. In other words, a mild periodate reaction is needed to leave the antigenic structure intact, but just enough to enable a coupling between protein and thus solid phase.

### Results:

LPS from Se, Sg, Sl and St was oxidized for 40 min at pH 5.5 using a range of sodium m-periodate concentrations. Following oxidation, LPS was coupled to a biosensor surface and immobilization efficiency and antigenic activities was monitored.

From FIG. 25 it is obvious that a higher oxidation grade of LPS from S. enteritidis gave rise to a corresponding higher coating level at the biosensor chip. However, despite the higher immobilization levels, the response from the antibody probing decreases as demonstrated in FIGS. 26 and 27. For this LPS type an optimum periodate concentration of 1.8 mM was determined.

In a similar way, the effects of oxidation on the immobilization and antigenic activity of LPS from S. goldcoast were tested (FIGS. 28 and 29). An identical optimum for the sodium periodate concentration was found at 1.8 mM. Similar results were obtained for S. livingstone (FIGS. 30 and 31). An optimum of 1.8 mM m-periodate was found here as well.

### Example 11

### Extraction of Lipopolysaccharides from Salmonella Spp.

### 0. Introduction

Salmonella is a gram-negative bacterium, and its outer membrane consists of various antigenic structures, including flagella, outer membrane proteins and Lipopolysaccharides (LPS). The molecule of LPS consists of a so-called lipid A part, which is embedded in the leaflet of the outer membrane, a core region and polysaccharide. The core region is composed of two or three heptoses and two or three residues of eight-carbon, negatively charged monosaccharides KDO. The core region links lipid A to the polysaccharides, which is also known as the O-side chain. This O-side chain is highly variable with respect to its length and composition between strains, but also within a strain influenced by growth conditions of the Salmonella. Despite variation, antigenic structures coded in the PS are unique for a certain Salmonella serovar. In fact, antigenic structures O3, O4, O6/7, O8, O9, O10 and O12 represent approximately 90% of known Salmonella serovars occurring on porcine products, in particular, Dutch abattoirs.

To detect a humoral response to O antigens as an indication of an exposure of farm animals to Salmonella, LPS can be used to probe the binding of raised antibodies to these biomolecules. For this purpose, LPS from S. typhimurium (O4, O5, O12), S. enteritidis (O9, O12), S. livingstone (O6/O7), S. goldcoast (O6, O8) and S. meleagridis (O3, O10) can be extracted.

An in-house extraction is paramount because LPS from only a limited number of Salmonella serovars is commercially available. Furthermore, in-house production can secure a continuous availability of LPS types for a successful antibody detection assay. The in-house extraction method described here for this purpose, is based on a protocol described by Staub Trichloroacetic acid (TCA) extracts LPS containing 1-10% protein contamination. This product is suitable for covalent immobilization of LPS to a carboxymethylated dextran layer coated on a gold metal surface of a biosensor chip (see SOP CHEMIE/A22 (Example 12)). This chip immobilized with LPS, in combination with a Biacore optical SPR biosensor system, can be used to trace Salmonella-LPS antibodies in sera also known as serology.

### 1. Scope and Field of Application

This method describes the extraction of LPS from several Salmonella serovars with the use of trichloric acetic acid. Extracted LPS is suitable for modifications to facilitate its immobilization on a carboxymethylated dextran surface.

### 2. References

Staub, A. M., Methods in Carbohydrate Chemistry, 5, 92 (1965)
SOP Chemie/A22: Immobilisation of Salmonella-derived LPS onto a biosensor chip (Biacore) and detection of serum antibodies reporting a current or past Salmonella infection (Example 12).
SOP Chemie/A23: Optimalisation of protein addition to LPS for immobilization and detection of serum antibodies (Example 13).

### 3. Definitions

c=concentration in % (m/v), % (v/v), mol/l or mmol/l as indicated.

### 5. Principle

Lipopolysaccharides (LPS) are produced by the extraction of Salmonella with the aid of trichloricacetic acid (TCA). Salmonella is cultured on and then collected from Brain Heart Infusion agar plates. After several washings steps with a saline solution and several centrifugation steps, TCA is added. The acidified suspension is incubated at a low temperature for three hours to solubilise LPS from bacterial cells. The suspension is centrifuged to remove cellular material and the pH is neutralized. LPS is then partly purified and concentrated by ethanol precipitation at low temperature. Finally, salts and ethanol are removed by dialysis, and remaining particles in the retained LPS-containing solution are, spun down by centrifugation. The supernatant is lyophilized and weighed to determine the recovery of produced LPS.

### 6. Reagents and Materials

During the procedure, unless stated otherwise, use only reagents of recognized analytical grade and only distilled water or water of equivalent purity.

Reference to a company is for information and identification only and does not imply a recommendation unless so stated.

### 6.1 Chemicals

### 6.1.1 Brilliant Green Agar (Oxoid, Basingstroke, England, CM329)

### 6.1.2 Brain Heart Infusion (Oxoid, CM225)

### 6.1.3 Brain Heart Infusion Agar (Oxoid, CM375)

6.1.4 Ethanol, absolute (Merck, Darmstad, Germany, 1.00983.2500)

### 6.1.5 Glycerol 87% (Merck, 1.04091.1000)

### 6.1.6 Nutrient Broth No 2 (Oxoid, 67)

6.1.7 Sodium chloride (Merck, 1.06404.1000)
6.1.8 Sodium hydroxide (Merck, 1.06498.1000)
6.1.9 Ethyleen glycol (Merck, 9621.2500))
6.1.10 Trichloroacetic acid (Merck, 1.00807.0250)
6.1.11 water was obtained from the Milli Q purification system (8.24)

### 6.2 Salmonella Agglutination Sera

6.2.1 anti-O4 (Pro-Lab diagnostics, Salmonella reference section of the Central Veterinary Laboratory, Weybridge, Great Britain)
6.2.2 anti-O5 (Pro-Lab diagnostics)
6.2.3 anti-O6, 7 (Pro-Lab diagnostics)
6.2.4 anti-O8 (Pro-Lab diagnostics)
6.2.5 anti-O9 (Pro-Lab diagnostics)
6.2.6 anti-O12 (Pro-Lab diagnostics)
6.2.7 anti-O Poly A-S (antisera to groups. A through S) (Pro-Lab diagnostics)
6.2.8 anti-O Poly E (antisera to factors O3, O10, O15, O19, O 34) (Pro-Lab diagnostics)

### 6.3 Bacterial Strains

6.3.1 Salmonella enteritidis (#23, phage type 1 strain RIVM, The Netherlands; 90-16-706)
6.3.2 Salmonella goldcoast (Division's working bank, Utrecht University, The Netherlands)
6.3.3 Salmonella livingstone (Division's working bank)
6.3.4 Salmonella melaegridis (Division's working bank)
6.3.5 Salmonella typhimurium X-193 (ASG, Lelystad)

### 6.4 Reagents

6.4.1 Brilliant Green agar (BGA) plates: Suspend 52 g of BGA (6.1.1) in 1.0 l water (6.1.11). Boil to dissolve the medium completely. Mix well and dispend 15 ml portions in petri dishes.
6.4.2 Brain Heart Infusion (BHI) broth: Dissolve 37 g of BHI broth (6.1.2) in 1.0 l water (6.1.11). Mix well, distribute into final containers and sterilize by autoclaving at 121° C. for 15 min.
6.4.3 Brain Heart Infusion Agar (BHIa): Suspend 47 g BHI agar (6.1.3) in 1.0 l water (6.1.11). Boil to dissolve the medium completely. Mix well and dispend 15 ml portions in petri dishes.
6.4.4 Cooling solution: Mix 1.0 l ethylene glycol (6.1.9) with 3 l water
6.4.5 Cold ethanol: Store 1 l ethanol (6.1.4) o/n in a freezer (−18° C.)
6.4.6 Glycerol 87% sterile: Autoclave 50 ml glycerol (6.1.5) at 121° C. for 15 min.
6.4.7 Nutrient broth (NB): Dissolve 25 g NB (6.1.6) in 1.0 l Water (6.1.11). Mix well, distribute into 100 ml flasks and sterilize by autoclaving at 121° C. for 15 min.
6.4.8 Saline (c=0.9% (m/v)): Dissolve 9 g sodium chloride (NaCl) (6.1.7) in 1.0 l water (6.1.11). Before use, cool the saline overnight in a refrigerator.
6.4.9 Trichloroacetic acid (TCA) solution, c=0.25 mol/l
6.4.10 Trichloroacetic acid (TCA) solution, c=0.50 mol/l
6.4.11 Sodium hydroxide (NaOH) solution, c=5.0 mol/l
6.4.12 Sodium hydroxide (NaOH) solution, c=0.10 mol/l

### 8. Procedure

### 8.1 Preparation Stock Culture

8.1.1 Make an isolate of Salmonella (6.3) by spreading one colony, or the content of an inoculation loop onto a BGA plate (6.4.1).
8.1.2 Incubate the plate (8.1.1) overnight at 37° C.
8.1.3 A single colony is picked from the plate (8.1.2) with an inoculation loop and suspended in 100 ml NB (6.4.7)
8.1.4 Incubate overnight at 37° C.
8.1.5 Add 50 ml glycerol (6.4.5) to 100 ml cultured NB medium (8.1.4)
8.1.6 Aliquot culture/glycerol mixture (8.1.5) into eleven 14 ml (volume of 12.5 ml) and twelve 1.5 ml (volume of 1 ml) sterile tubes.
8.1.7 One of the 1.5 ml tubes is marked as standard bank. The contents of this tube will only be used to prepare more aliquots of 1 and 12.5 ml via the method here described (8.1).
8.1.8 Quickly freeze the aliquots at −80° C. (8.1.6) and store until use.

### 8.2 Determination of Salmonella Isolate Purity by Agglutination

8.2.1 Pipette 25 μl sterile saline (6.4.8) onto a glass slide.
8.2.2 Suspend one colony from the plate cultured Salmonella (8.1.2) in the saline (6.4.8).
8.2.3 Add a single drop of agglutination serum (6.2) using the facilitated container drop system
8.2.4 Mix sera and suspension by gently tilting the slide back and forth for two min.
8.2.5 Determine agglutination by looking for aggregation formation in front of a black background.
8.2.6 Disclose the identity of Salmonella strain by compare the results of aggregation with Table 37.

8.2.7 When the identity of the cultured strain differs from predicted aggregation in Table 37, it can be concluded that the tested culture was not (exclusively) composed of the expected Salmonella serotype. In such case, a new stock culture has to be prepared.

8.3 Extraction method
8.3.1 Prepare 120 BHI agar plates (6.4.3).
8.3.2 Thaw a 12.5 ml tube with Salmonella stock culture (8.1.8).
8.3.3 Spread 100 μl stock culture (8.1.8) on each plate (8.3.1) with a spatula.
8.3.4 Incubate overnight at 37° C.
8.3.5 Fill a 15 l container with 10 l water (6.1.11), and cool to 4-8° C. in a refrigerator until further use.
8.3.6 Weigh six empty centrifugation tubes and note their weight on the quality sheet (see FIG. 32.).
8.3.7 Put milliQ (6.1.11) and TCA (6.4.9, 6.4.10) containers in prepared ice salt bath.
8.3.8 Take twenty plates (8.3.4) overnight incubated plates and add 1 ml of saline to each plate.
8.3.9 Harvest the bacteria by lightly scraping (making round movements) a Drigalski spatula over the agar, making a suspension of the bacteria in saline.
8.3.10 Collect the suspension in one of the six pre-weighed centrifugation tubes (8.3.6).
8.3.11 Wash each plate (8.3.9) twice with 2 ml saline (6.4.8) solution.
8.3.12 Combine the suspensions (8.3.11) with the contents of the centrifugation tube (8.3.10).
8.3.13 Repeat steps 8.3.8 through 8.3.12 5 times for the remaining plates (8.3.4).
8.3.14 Add 100 ml saline (6.4.8) to each centrifugation tube.
8.3.15 Tare the tubes (8.3.14)

### 8.3.16 Centrifuge for 15 min at 10,000×g and 4° C.

8.3.17 Decant the supernatant in a waste container.
8.3.18 Suspend each bacterial pellet in 10 ml saline (6.4.8) until a smooth suspension is formed.
8.3.19 Add 75 ml saline (6.4.8) to each centrifuge tube.
8.3.20 Repeat steps 8.3.14 to 8.3.19 once.
8.3.21 Repeat steps 8.3.15 to 8.3.17 once.
8.3.22 Weigh the tubes with bacterial pellet (8.3.21) and note weigh results on the quality sheet (see FIG. 32)
8.3.23 Determine the mass (=m) of the deposited bacteria by subtracting the weight of the empty (8.3.6) with that of the cell-containing tubes (8.3.22).
8.3.24 Suspend the bacterial pellets (8.3.22) with x ml (for determination x, see Table 38) of water (6.1.11) by repeatedly drawing in and washing with a 10 ml pipette.
8.3.25 Combine the suspensions of two centrifugation tubes in one tube.
8.3.26 Repeat 8.3.25 for the remaining centrifugation tubes.
8.3.27 Add x ml of y M TCA (6.4.10) (see Table 38 for values x and y).
8.3.28 Insert stirring rods in each of the suspensions (8.3.27).
8.3.29 Stir the suspensions (8.3.28) for 3 h at 4° C. on a magnetic stirrer.
8.3.30 Remove the stirring rods.
8.3.31 Centrifuge the suspensions for 30 min at 20,000×g and 4° C.
8.3.32 Collect the supernatants of the centrifugation tubes in a 500-ml beaker.
8.3.33 Adjust the pH of the supernatant with 5 M NaOH (6.4.11) and 0.10 M NaOH (6.4.12) to pH 6.5.
8.3.34 Determine the volume (v) of the pH adjusted supernatant (8.3.33)
8.3.35. Cool the supernatant to freezing point by putting the filled flasks (8.3.34) in a −18° C. freezer for 30 min
8.3.36 Add 2*e ml (for values of e see Table 38) freeze-cold ethanol (6.4.5).
8.3.37 Cool the solution overnight at −4° C.
8.3.38 Dispense the solution in six centrifuge tubes.
8.3.39 Centrifuge the solution for 30 min at 20,000×g and −4° C.
8.3.40 Cut three parts (each 10 cm of length) from the dialysis tube
8.3.41 Wash the dialysis tubes (8.3.40) briefly with water (6.1.11) and keep them wet in water (6.1.11) until further use
8.3.42 Clip a membrane clampat one end of the dialysis tube (8.3.41), leaving 1 cm tubing free.
8.3.43 Decant the supernatant (8.3.39) in a waste bottle.
8.3.44 Remove the remaining supernatant from the centrifuge tubes with the help of a pipette.
8.3.45 Add one ml of water (6.1.11) to each centrifuge tube (8.3.44).
8.3.46 Suspend the pellets by drawing in and washing out with a one-ml pipette.
8.3.47 Fill one of the prepared dialysis bags (8.3.42) with the re-suspended pellets (8.3.46) of two centrifuge tubes.
8.3.48 Wash each of the tubes with (z−1)/6 ml (for the value of z, see Table 38) water (6.1.11) and combine the wash with the contents of the dialysis bag (8.3.47).
8.3.49 Clip another clamp (6.10) on top of the filled dialysis tube (8.3.48) leaving a small air bubble between solution and clamp.
8.3.50 Repeat the steps 8.3.42 to 8.3.49 for the remaining centrifuge tubes.
8.3.51 Place the three filled dialysis tubes in pre-cooled water (8.3.5) at 4° C.
8.3.52 Incubate dialyse the contents of the tubes (8.3.51) for two days under continuous gentle stirring conditions at 4° C. on a magnetic stirrer.
8.3.53 Refresh the dialysate at least twice, by exchange the water with 71 fresh, precooled water (6.1.11).
8.3.54 Collect the contents of the dialysis tubes in a 50-ml container
8.3.55 Divide the collected volume (8.3.54) in centrifuge tubes
8.3.56 Centrifuge the tubes for 20,000×g for 30 min at 4° C.
8.3.57 Weigh an empty 50-ml container (7.8) on an analytic balance (without cap) and note its weight on the quality sheet (see FIG. 32)
8.3.58 Collect the supernatant (8.3.56) in pre-weighed container (8.3.57).
8.3.59 Freeze the supernatants in an −80° C. freezer (6.15).
8.3.60 Lyophilize (6.15) the frozen supernatants (8.3.59) until a dry white crystal structure is observed.

Weigh lyophilized LPS-holding container (8.3.60) on an analytical balance and note the resulting mass on the quality sheet (see FIG. 32)

8.3.62 Calculate the yield of LPS: (weight LPS (8.3.61)−weight container (8.3.57))/total mass wet cells (8.3.23)*100%
8.3.63 Store lyophilized LPS powder in a closed container at 4° C. until further use.

An overview of the procedure is depicted in FIG. 33.

TABLE 37
Check board for agglutination readings to disclose the identity of the
present bacteria in terms of Salmonella species and Salmonella serovar

| | Agglutination sera | | | | | | | |
|---|---|---|---|---|---|---|---|---|
| | | | | | | | [symbol] A-S | [symbol] y E |
| [symbol] ella strain | [symbol] .1) | [symbol] .2) | (7.2.3) | [symbol] .4) | [symbol] .5) | (7.2.6) | 2.7) | 2.8) |
| S. enteritidis | − | − | − | − | + | + | + | − |
| (6.3.1) | | | | | | | | |
| S. goldcoast | − | − | + | + | − | − | + | − |
| (6.3.2) | | | | | | | | |
| S. livingstone | − | − | + | − | − | − | + | − |
| (6.3.3) | | | | | | | | |
| S. melaegridis | − | − | − | − | − | − | + | + |
| (6.3.4) | | | | | | | | |
| S. typhimurium | + | + | − | − | − | + | + | − |
| (7.3.4) | | | | | | | | |

Legend:
+ = Aggregation formation, agglutination positive
− = No aggregation formation, agglutination negative [symbol]
indicates data missing or illegible when filed

TABLE 38
Conversion table to determine volumes of water (x), TCA (x), ethanol
(e) and TCA concentration (y) for extraction and precipitation, and
volume of water (z) for dialysis procedures to isolate and purify LPS
from Salmonella. Letters noted: m refers to mass (9.3.23),
whereas v refers to pH adjusted supernatant volume (9.3.34).

| | Extraction | | | |
|---|---|---|---|---|
| | x ml water | | | |
| | (6.1.11) and | | | |
| | x ml | | Precipitation | Dialysis |
| | TCA(6.4.9, | | e ml ethanol | z ml water |
| Strain | 6.4.10) | y M TCA | (6.4.5) | (6.1.11) |
| S. enteritidis | x = m * 5 | 0.25 (6.4.9) | e = 2 * v | z = x * |
| S. goldcoast | | 0.5 (6.4.10) | | 0.1 |
| S. livingstone | | 0.5 (6.4.10) | | |
| S. melaegridis | | n.e.y. | | |
| S. typhimurium | | 0.5 (6.4.10) | | |

n.e.y . . . : not established yet.

### Example 12

### Immobilisation of salmonella-Derived LPS onto a Biosensor Chip (Biacore) and Detection of Serum Antibodies Reporting a Current or Past Salmonella Infection

### 1. Introduction

After extraction and isolation of carefully chosen LPS (see SOP CHEMIE/A21 (Example 11)), antigen-containing LPS is coupled covalently to a biosensor chip surface to monitor serologically samples for the presence of anti-Salmonella antibodies through their binding to the immobilized antigen-containing LPS on the chip surface (see SOP CHEMIE/A23 (Example 13)). This SOP describes the method for oxidation, immobilization of LPS onto the biosensor chip (BIACORE) and the analysis of antibodies in sera.

### 2. Scope and Field of Application

To analyze serum samples from chicken for the presence of anti-Salmonella antibodies reacting with O4, 5, 6, 7, 8, 9 and 12 somatic antigens.

### 3. References

Concentration Analysis Handbook, Version AA, December 2001, Biacore AB, Uppsala, Sweden
BIAapplications Handbook, version AB (reprinted 1998), Biacore http://www.jp.amershambiosciences.com/tech_support/manual/pdf/dnapuri/52207400af.pdf
SOP Chemie/A21: Extraction of lipopolysaccharides from Salmonella spp. (Example 11)
SOP Chemie/A23: Optimalisation of protein addition to LPS for immobilization and detection of serum antibodies (Example 13).

### 4. Definitions

c=concentration in % (m/v), % (v/v), mol/l or mmol/l as indicated.

### 6. Principle

LPS is oxidized in the presence of a protein facilitated by sodium periodate. The LPS-protein solution is desalted using a NAP-5 column. The LPS-protein complex is immobilized on a CM5-chip after activation of the carboxymethyl dextran layer on a biosensor chip with the aid of 1-ethyl-3-(3-dimethylaminopropyl)carbodiimide hydrochloride (EDC) and N-hydroxysuccinimide (NHS) and carbohydrazide. Bound LPS is then stabilized with sodium cyanoborohydride. Prior to routinely use, the performance of biosensor chip-conjugated LPS to bind anti-Salmonella antibodies is assessed using a panel of reference polyclonal agglutination sera.

### 7 Reactions

### 7.1 Oxidation of Carbohydrate Moiety

See example 9.

### 7.2 Conjugation to Protein

See example 9

### 7.3 Immobilization to Sensor Surface

See example 9.

### 8. Reagents and Materials

In the complete procedure only reagents of recognized analytical grade and only distilled water or water of equivalent purity are used, unless stated otherwise. Reference to a company is for information and identification only and does not imply a recommendation unless so stated.

### 8.1 Chemicals

8.1.1 Acetic acid (J.T. Baker, Deventer, The Netherlands)
8.1.2 Amine coupling kit (Biacore AB, Uppsala, Sweden) consisting of:
8.1.2.1 Vial containing 115 mg N-hydroxysuccinimide (NHS)
8.1.2.2 Vial containing 750 mg 1-ethyl-3-(3-dimethlylaminopropyl)carbodiimide hydrochloride (EDC)
8.1.2.3 Vial containing 10.5 ml, c=1 mol/l, ethanolamine hydrochloride—sodium hydroxide pH 8.5
8.1.3 CHAPS (Plus one, Pharmacia Biotech, Uppsala, Sweden)

### 8.1.4 Carbohydrazide, CN₄H₆O (Fluka Chemie GmbH, Buchs, Switzerland)

8.1.5 Carboxymethyl-dextran sodium salt (Fluka)
8.1.6 Glycine, c=10 mmol/l pH 1.5 (Biacore)
8.1.7 Guanidine hydrochloride (Calbiochem, San Diego, Calif., USA)
8.1.8 HBS-EP buffer (Biacore) containing HEPES buffer, c=10 mmol/l, pH 7.4, sodium hydrochloride, c=150 mmol/l, EDTA, c=3 mmol/l and surfactant P20, c=0.005% (v/v).
8.1.9 Salmonella anti group specific, monoclonal test reagents:
8.1.9.1 anti-Salmonella gr. B (SIFIN, Berlin, Germany), contains mAb Anti-O4, O5, O27
8.1.9.2 anti-Salmonella gr. C(SIFIN), contains mAb Anti-O7, O8
8.1.9.3 anti-Salmonella gr. D (SIFIN), contains mAb Anti-O9, Vi
8.1.9.4 anti-Salmonella gr. E (SIFIN), contains mAb Anti-O3, O19
8.1.10 Salmonella monovalent ‘O’ somatic anti sera:
8.1.10.1 anti-O4 (Pro-Lab diagnostics, Salmonella reference section of the Central Veterinary Laboratory, Weybridge, Great Britain)
8.1.10.2 anti-O5 (Pro-Lab diagnostics)
8.1.10.3 anti-O6,7 (Pro-Lab diagnostics)
8.1.10.4 anti-O8 (Pro-Lab diagnostics)
8.1.10.5 anti-O9 (Pro-Lab diagnostics)
8:1.10.6 anti-O10 (Pro-Lab diagnostics)
8.1.10.7 anti-O12 (Pro-Lab diagnostics)
8.1.10:8 anti-O19 (Pro-Lab diagnostics)
8.1.10.9 anti-O Poly E (O3, O10, O15, O19, O34; Pro-Lab diagnostics)
8.1.11 Salmonella polyvalent ‘O’ somatic anti sera:
8.1.11.1 anti-O Poly A-S (O2, O3, O4, O5, O6, 7, O8, O9, O10, O11, O12, O13, O15, O16, O17, O18, O19, O20, O21, O22, O23, O28, O30, O34, O35, O38, O40, O41; Pro-Lab. diagnostics)
8.1.12 Salmonella LPS, in-house isolated LPS by TCA extraction (SOP CHEMIE/A21 (Example 11)) prepared from the Salmonella bacteria serovars enteriditis (Se), goldcoast (Sg), livingstone (Sl), meleagridis (Sm) and typhimurium (St) with protein (SOP CHEMIE/A23, example 13)
8.1.12.1 Aliquots of 0.5 mg LPS are stored at +4° C. or at lower temperature.
8.1.13 Avian reference sera
8.1.13.1 SPF-CH, SPF serum referred to as negative control serum (Animal Health Service Ltd. (GD), Deventer, The Netherlands)
8.1.13.2 EIA-SE, Salmonella enteritidis positive control serum from chicken for use in ELISA (GD)
8.1.13.3 EIA-ST, Salmonella typhimurium positive control serum from chicken for use in ELISA (GD)
8.1.13.4 SPA-PG, Salmonella pullorum positive control serum from chicken for use in ELISA (GD)
8.1.13.5 CH-SI, Salmonella infantis positive control serum from chicken for use in ELISA (GD)
8.1.14 Sodium acetate trihydrate (J.T. Baker, Phillipsburgh, N.J., USA)
8.1.15 Sodium chloride (Merck, Darmstadt, Germany)
8.1.16 Sodium cyanoborohydride (NaCNBH₃) (Fluka)
8.1.17 Sodium hydroxide, c=50 mmol/l (Biacore)
8.1.18 Sodium periodate (Sigma Chemical Comp., St. Louis, Mo., USA)

### 8.1.19 Triton X-100 (Sigma)

### 8.1.20 Tween 20 (Sigma)

### 8.1.21 Tween 80 (Sigma)

8.1.22 Water is obtained from a Milli Q water purification system (MilliQplus)

### 8.2 Solutions

8.2.1 Acetic acid solution, c=0.1 g/ml
8.2.2 Acetate buffer solution, c=10 mmol/l, pH 4.0
8.2.3 Acetate buffer solution, c=1.0 mol/l, pH 5.5
8.2.4 Acetate buffer solution, c=100 mmol/l, pH 5.5
8.2.5 Carbohydrazide solution, c=100 mmol/l
8.2.6 Carbohydrazide solution, c=5 mmol/l
8.2.7 CHAPS solution, c=0.05% (m/v): Dissolve 0.02 g (8.2.3) in 40 ml HBS-EP (8.2.8).
8.2.8 Detergents solution, c=0.3% (m/v): Dissolve 0.3 g of CHAPS (8.2.3), 0.3 g Tween 20 (8.2.21), 0.3 g Tween 80 (8.2.22) and 0.3 g Triton X-100 (8.2.20) in 100 ml water.
8.2.9 EDC-solution: reconstitute EDC (8.1.2.2) in 10.0 ml water.
8.2.9.1 Fractions of 100 μl of this solution (8.3.9) are stored in polypropylene tube (9.12) at −18° C. or at lower temperature. The aliquots are stable for two months.
8.2.9.2 Before use: Thaw frozen aliquots and agitate them gently to ensure homogeneous solutions.
8.2.10 Ethanolamine solution: Pipette 200 μl c=1 mol/l ehthanolamine solution (8.1.2.3) in a polypropylene tube
8.2.11 Guanidine solution, c=6 mol/l: Dissolve 17.18 g guanidine hydrochloride (8.2.7) in 10 ml detergents solution (8.3.8) and adjust volume to 30 ml with glycine buffer solution (8.2.6)
8.2.12 NHS-solution: reconstitute NHS (8.1.2.1) in 10.0 ml water.
8.2.12.1 Fractionate 100-μl aliquots of this solution (8.3.12) in polypropylene tube (9.12). Store at −18° C. or at lower temperature. The aliquots are stable for two months.
8.2.12.2 Before use: Thaw frozen aliquots and agitate them gently to ensure homogeneous solutions.
8.2.13 Sample dilution buffer: Dissolve 2 g carboxymethyl-dextran sodium salt (8.2.5), 9.97 g sodium chloride (8.2.15) and 0.1 g Tween 80 (8.2.22) in 200 ml HBS-EP (8.2.8).
8.2.14 Sodium cyanoborohydride, c=1.00 mol/l
8.2.15 Sodium cyanoborohydride, c=100 mmol/l
8.2.16 Sodium hydroxide, c=5 mmol/l
8.2.17 Sodium periodate solution, c=100 mmol/l
8.2.18 Sodium periodate ‘ready to use’: Pipette 100 μl of sodium periodate solution, c=100 mmol/l (8.3.17) in a 1.4 ml polypropylene tube and dry with a centrifugal evaporator.
8.2.19 Sodium periodate solution, c=50 mmol/l: Dissolve sodium periodate ‘ready to use’ (8.3.18) in 200 μl acetate solution, c=100 mmol/l pH 5.5 (8.3.4).

### 8.3 Standard Reference Solution

8.3.1 Salmonella anti-O sera: Dilute 20 μl of each serum (8.2.10 and 8.2.11) in 380 μl sample dilution buffer (8.3.13) in a micro titerplate with the exception of anti-O5 serum: 2 μl of this serum (8.2.10.2) is diluted in 400 μl sample dilution buffer (8.3.13).

8.3.2 Salmonella anti-group specific test reagents: Dilute 4 μl of each serum (8.2.9.1, 8.2.9.2, 8.2.9.3 and 8.2.9.4) in 395 μl sample dilution buffer (8.3.13)
8.3.3 Avian reference sera: Dilute 6 μl sera (8.2.13.1 and 8.2.13.3) in 295 μl sample dilution buffer (8.3.13) in a micro titerplate and dilute 3 μl sera (8.2.13.2 and 8.2.13.4) in 295 μl sample dilution buffer (8.3.13)
8.3.4 Shake. Prepare just before use.

### 8.4 Auxiliary Materials

8.4.1 NAP-5 column (0.5 ml, Sephadex G-25, Amersham Biosciences).
8.4.2 CM5 chips (Biacore).

### 10. Software

The biosensor apparatus is operated with Biacore 3000 control software 4.1 (1999-2003).

### 11. Procedure

### 11.1 Oxidation and Desalting of LPS Solution

### 11.1.1 OXIDATION

11.1.1.1 Add 500 μl acetate buffer pH 5.5 (8.3.4) to the LPS (8.2.12)
11.1.1.2 Vortex thoroughly until the pellet is solved.
11.1.1.3 Sonicate the solution for 10 minutes and judge the solution for its clearance.
11.1.1.4 When clearance is not satisfactory continue sonication until a clear (convalescent) solution is obtained.
11.1.1.5 Add 20 periodate solution (8.3.19) to the LPS solution (11.1.2.4)
11.1.1.6 Vortex the solution (11.1.2.5)
11.1.1.7 Incubate on ice for 40 min protected from light.
11.1.1.8 Quench oxidation by desalting the solution (11.1.2.6) as described in 11.1.3

### 11.1.2 Desalting

11.1.2.1 Place NAP-5 column(s) (8.5.1) on manifold.
11.1.2.2 Condition the column(s) (11.1.3.1) by passing three 3-ml portions of acetate buffer (8.3.2) over the column bed on a flow generated by gravity only. Allow the buffer to enter the gel bed completely.
11.1.2.3 Pipette 0.5 ml oxidized LPS solution (11.1.2.8) on the column. Allow the sample to enter the gel bed completely.
11.1.2.4 Elute oxidized LPS with 1 ml of acetate buffer (8.3.2). Collect eluate in a 5-ml glass tube.
11.1.2.5 Vortex the solution (11.1.3.4) for 10 s.
11.1.2.6 When not immediately used (11.1.3.5) store samples at 4° C. to 7° C.
11.1.2.7 Prior to immobilization, the LPS-containing solution is diluted as indicated in the following Table 39.

TABLE 39

| | | End volume (μl) make with |
|---|---|---|
| LPS spp | μl stock solution | acetate buffer, |
| (8.2.12) | (11.1.3.6) | pH 4.0 (8.3.2) |
| Se | 25 | 200 |
| Sg | 100 | 200 |
| Sl | 100 | 200 |
| St | 100 | 200 |
| Sm | 12.5 | 200 |

### 11.2 Immobilization

### 11.2.1 Preparation

11.2.1.1 Thaw a portion of EDC (8.3.9.1)
11.2.1.2 Thaw a portion of NHS solution (8.3.12.1).
11.2.1.3 Place the rack Thermo A in the right rack position (R2) and the Reagent rack in the middle (RR)
11.2.1.4 Command: Dock a CM5 chip (8.5.2) and prime with HBS-EP buffer (8.2.8).
11.2.1.5 Place EDC solution (11.2.1.1) in position R2A1
11.2.1.6 Place the NHS solution (11.2.1.2) in position R2A2.
11.2.1.7 Place an empty tube (9.12) in position R2A3.
11.2.1.8 Place the carbohydrazide solution (8.2.6) in position R2A4.
11.2.1.9 Place the ethanolamine solution (8.2.10) in position R2A5.
11.2.1.10 Place the solution with oxidized LPS (11.1.2.7) in position R2A6.
11.2.1.11 Place the cyanoborohydride solution (8.2.15) in position R2A7.
11.2.1.12 Place the 6 M guanidine solution (8.2.11) in glass vial (9.10) in position RR2
11.2.1.13 Place the CHAPS solution (8.2.7) in glass vial (9.10) in position RR4
11.2.2 Immobilization of the CM5 chip
11.2.3 File (see FIG. 35.)→New application wizard
11.2.3.1 Open Template→Search for file: Wizard immobilisation see FIG. 36)

### 11.2.3.2 Fill in: Notebook (see FIG. 38)

11.2.3.3 Run, next en start
11.2.3.4 Note: To see which instruction are in the wizard do Edit in stead off Run
11.2.4 Save the sensorgram. The result files are saved with the default extension ‘.blr’
11.2.5 The chip is ready for testing Salmonella antibodies in sera.

TABLE 40
Typical immobilization levels of lps

| LPS | Immobilization level in |
|---|---|
| spp | RU (Standard deviation)¹ |
| Se | 2365 (959) |
| Sg | 8609 (1969) |
| Sl | 10953 (2135) |
| St | 4836 (1023) |

### 11.3 Detection of Anti-Salmonella Antibodies

11.3.1 Make the Biacore operational with an appropriate CM5 chip.
11.3.2 File (see FIG. 36)→New application wizard
11.3.2 Open Template→Search for file: Wizard control chip immobilization (see FIG. 37)

### 11.3.2.2 Fill in: Notebook (see FIG. 38)

11.3.2.3 Run, next en start
11.3.2.4 Note: To see which instruction are in the wizard do. Edit in stead off Run
11.3.3 Save the sensorgram. The result files are saved with the default extension ‘.blr’

Typical sensorgram for immobilization of LPS is depicted in FIG. 39, while a typical sensorgram for to analysis of an antiserum is depicted in FIG. 40. Typical responses are listed in Table 41.

TABLE 41
Typical responses of salmonella antibodies sera

| | Anti Salmonella sera | | | | | | | | |
|---|---|---|---|---|---|---|---|---|---|
| LPS spp | O4 | O5 | O6, 7 | O8 | O9 | O12 | O poly E | O poly A-S | Sample dilution buffer |
| Se | 9 | −1 | 10 | 4 | 240 | 243 | 2 | 296 | −8 |
| Sg | 4 | −7 | 606 | 453 | −1 | 23 | 4 | 194 | −3 |
| Sl | 4 | −3 | 263 | −1 | 2 | 2 | 5 | 105 | −8 |
| St | 460 | 526 | 16 | 5 | 5 | 224 | 6 | 287 | −5 |

### Example 13

### Optimalisation of Protein Addition to LPS for Immobilization and Detection of Serum Antibodies

### 0. Introduction

Recent studies indicate that when protein is added to LPS before oxidation, the immobilization to a carboxymethylated dextrane gold layer is made possible and in some cases is improved. Following, serological responses are also made possible and are improved. The optimum of serological responses depends on the percentage of protein added to LPS.

This protein effect can be obtained by addition of hemoglobin. Hemoglobin is a naturally occurring protein, which can be found in all warm-blooded vertebrates. Therefore cross-reacting anti hemoglobin antibodies in sera are not expected.

### 1. Scope and Field of Application

This method describes the addition of an amount of hemoglobin to lipolysaccharides (LPS) produced through trichloric acid extraction (see SOP Chemie/A21: Extraction and isolation of Lipopolysaccharides (Example 11)). The optimal hemoglobin percentage is defined as a reaction mixture giving high immobilization levels in combination with maximum serological reaction of positive control sera. Furthermore, the production and storage of LPS reaction mixtures, ready to be used, for immobilization on sensor chips is described.

### 2. References

SOP Chemie/A21: Extraction and isolation of Lipopolysaccharides (version 3; Example 11)
SOP CHEMIE/A22: Immobilization of salmonella derived LPS onto a biosensor chip (BIACORE) and detection of serum antibodies reporting a current or past salmonella infection (version 3; Example 12)

### 4. Principle

In SOP Chemie/A21 (Example 11), the production of Salmonella spp. lipopolysaccharides (LPS) is described. Extracted LPS used as a ligand in an analytical analysis performed on a Biacore 3000 system to detect anti Salmonella antibodies in sera derived from pigs and chicken (see SOP CHEMIE/A22 (Example 12)). To improve immobilization of LPS, hemoglobin is added before oxidation. To produce a large stock of material to give reproducible immobilization levels, serological data and method performance, LPS is fortified with hemoglobin, divided in aliquots and dried before storage at 4° C. To immobilize a chip, one of the aliquots is batch-wise oxidized and immobilized.

### 5. Reagents and Materials

### 5.1 Chemicals

5.1.1 Acetic acid (J.T. Baker, Deventer, The Netherlands)
5.1.2 Hemoglobin, porcine (Sigma-Aldrich, Zwijndrecht, The Netherlands)
5.1.3 MilliQ water
5.1.4 Sodium acetate trihydrate (J.T. Baker, Phillipsburgh, N.J., USA)

### 5.2 Salmonella Agglutination Sera

5.2.1 anti-O4 (Pro-Lab diagnostics, Salmonella reference section of the Central Veterinary Laboratory, Weybridge, United Kingdom)
5.2.2 anti-O5 (Pro-Lab diagnostics)
5.2.3 anti-O6,7 (Pro-Lab diagnostics)
5.2.4 anti-O8 (Pro-Lab diagnostics)
5.2.5 anti-O9 (Pro-Lab diagnostics)
5.2.6 anti-O10 (Pro-Lab diagnostics)
5.2.7 anti-O12 (Pro-Lab diagnostics)
5.2.8 anti-O19 (Pro-Lab diagnostics)
5.2.9 anti-O Poly A-S (antisera to groups A through S) (Pro-Lab diagnostics)
5.2.10 anti-O Poly E (antisera to factors O3, O10, O15, O19, O34) (Pro-Lab diagnostics)

### 5.3 Group Specific Salmonella Antisera

5.3.1 Enteroclon anti-Salmonella group B (Sifin, Berlin, Germany)
5.3.2 Enteroclon anti-Salmonella group C (Sifin)
5.3.3 Enteroclon anti-Salmonella group D (Sifin)
5.3.4 Enteroclon anti-Salmonella group E (Sifin

### 5.4 Avian Reference Sera

5.4.1 SPF-CH, specific pathogen free (SPF) negative control serum (Animal Health Service Ltd. (GD), Deventer, The Netherlands)
5.4.2 EIA-SE, chicken Salmonella enteritidis positive control for use in ELISA (GD)
5.4.3 EIA-ST, chicken Salmonella typhimurium positive control for use in ELISA (GD)
5.4.4 SPA-PG, chicken Salmonella pullorum positive control for use in ELISA (GD)
5.4.5 CH-SI, chicken Salmonella infantis positive control for use in ELISA (GD)

### 5.5 Swine Reference Sera

5.5.1 Sw-Liv, swine Salmonella livingstone positive control serum in ELISA (GD)
5.5.2 Sw-Typ, swine Salmonella typhimurium positive control serum in ELISA (GD)
5.5.3 Sw-APP, swine Actinobaccilus Pleuropneumoniae positive control serum in ELISA (GD)

### 5.6 Lipopolysaccharides

Lipopolysaccharides (LPS) are extracted, lyophilized and stored as described in SOP Chemie/A21 (Example 11).

5.6.1 Salmonella enteritidis LPS
5.6.2 Salmonella goldcoast LPS
5.6.3 Salmonella livingstone LPS
5.6.4 Salmonella meleagridis LPS
5.6.5 Salmonella typhimurium LPS

### 5.7 Reagents

5.7.1 Acetate buffer solution, c=10 mmol/l, pH 4.0 5.7.2 Acetate buffer solution, c=1.0 mol/l, pH 5.5 5.7.3 Hemoglobin stock solution, 5 mg/ml

### 5.8 Auxiliary Materials

5.8.1 CM5 chips (Biacore AB, Uppsala, Sweden).

### 7. Procedure

### 7.1 Production of Stock Solution of Lipopolysaccharides

7.1.1 Collect the produced LPS (see SOP Chemie/A21 (Example 11)) from the refrigerator and let it acclimatize to room temperature.
7.1.2 Retrieve the weight of produced LPS (7.1.1) in the tube from the quality data sheet (see SOP Chemie/A21 (Example 11)).
7.1.3 Calculate the volume of mQ to be added to LPS using Formulae 1 (9.1).
7.1.4 Add the calculated volume of mQ (7.1.3) to the LPS tube (7.1.2) (end-concentration LPS: 5 mg/ml).
7.1.5 Vortex thoroughly until all powder is solved.
7.1.6 Sonicate the solution for 10 min and judge the solution for its clearance.
7.1.7 When clearance (7.1.6) is not satisfactory continue sonication (6.6) until a clear (convalescent) solution is obtained.

### 7.2 Addition of Hemoglobin

7.2.1 Prepare four (one for each flowchannel) LPS solution (7.17) dilutions in sodium acetate buffer with variable hemoglobin contents as described in Table 42 (8) in a glass tube (6.4).
7.2.2 The choice of relative hemoglobin starting amounts added to each newly prepared LPS extraction batch are given in Table 43 (8).
7.2.3 Fill up to a total volume of 500 μl with mQ (5.1.3) as described in Table 42 (8).
7.3 Oxidation and Desalting (see SOP Chemie/A22; chapter 10.1 (Example 12)
7.3.1 Start Oxidation from Point 10.1.1.2.
7.4 Immobilization (see SOP Chemie/A22, chapter 10.2 (Example 12)

NOTE: Immobilize oxidized LPS fortified with four different relative amounts of hemoglobin (7.3) on one a CM5 chip (5.8.1) so each flow channel represents a different relative amount.

### 7.5 Detection of Anti-Salmonella Antibodies (See SOP Chemie/A22, Chapter 10.3 (Example 12))

### 7.6 Determination of Optimal Hemoglobin Percentage

7.6.1 Calculate the mean and standard deviation of the 5 relative responses of each of the agglutination sera listed in (5.2) and the group specific Salmonella anti-sera listed in (5.3) per flow channel.
7.6.2 Create a clustered column graph with on the x-axis the names of the agglutination and group specific Salmonella anti-sera, and on the y-axis the mean of the relative response units for all the different relative amounts of hemoglobin (7.6.1) (see for an example: FIG. 41).
7.6.3 Calculate the mean and standard deviation of the 4 relative responses of each of the avian reference control sera listed in (5.4) and the swine reference sera listed in (5.5) per flow channel.
7.6.4 Create a clustered column graph with on the x-axis the names of the avian control and the swine control sera, and on the y-axis the mean of the relative response units for all the different percentages of hemoglobin (7.6.3) (see for an example: FIG. 42).
7.6.5 Add Y-error bars to both clustered graphs (7.6.2, 7.6.4) by using the standard deviation values for each x-axis column (see for an example: FIG. 41 or 42).
7.6.6 Copy the calculated means of the selected positive expected sera (see Tables 44 to 46 (8)) and the negative SPF chicken sera of all measured relative hemoglobin amounts in a new table (see for an example Table 47 (8).
7.6.7 Subtract the responses of SPF chicken sera (7.6.6) from the responses of the expected positive sera (7.6.6) (see for an example: Table 48 (8)).
7.6.8 Determine the highest response per positive serum per relative hemoglobin amount and give this a value of 10 (see for an example Table 49 (8)).
7.6.9 Calculate for the rest of the flow channels the relative values by using formulae 2a (9.2) (see for an example Table 49 (8)).
7.6.10 Calculate the sum of all relative values per hemoglobin percentage (see for an example Table 49 (8)).
7.6.11 The optimal hemoglobin percentage (for the four percentages compared) is determined by the highest sum score in the four flow channel/hemoglobin percentages.
7.6.12 When the optimal relative hemoglobin amounts (7.6.11) is the highest or lowest hemoglobin amounts compared, steps 7.2 to 7.6.11 have to be repeated with the following conditions.
7.6.12.1 In case of the lowest hemoglobin amounts for Sg, Sm and Sg (20%) is the most optimal, the amounts of 20% and 30% are repeated in addition to 0% and 10% hemoglobin.
7.6.12.2 In case of the highest relative hemoglobin amounts for Se and St is most optimal, the amounts 20 and 30% are repeated in addition to 40 and 50% hemoglobin.
7.6.12.3 In case the highest relative hemoglobin amounts for Sg, Sm and Sl is most optimal, the percentages 40 and 50% are repeated in addition to 60 and 70% hemoglobin.
7.6.13 An optimum of hemoglobin percentage is reached:
7.6.13.1 When the sum of relative values (7.6.8), per four compared relative hemoglobin amounts, has the highest value.
7.6.13.2 In the range of compared hemoglobin where the highest value is detected, a higher and a lower level of hemoglobin addition is also determined.

### 7.7 Preparation of Hemoglobin Added Vacuum, Dried LPS Stock

7.7.1 The volume of the remaining 5 mg/ml LPS solution after determination of optimal hemoglobin amount is calculated by subtracting tube weight plus LPS solution (7.1.7) by the initial empty tube weight read from the quality data sheet (see SOP Chemie/A21 (Example 11).
7.7.2 The amount of remaining LPS in calculated using Formulae 3 (9.3).
7.7.3 Calculate the amount of hemoglobin to be added to LPS using Formulae 4 (9.4).
7.7.4 Add the calculated amount of hemoglobin (7.7.1) to the remaining LPS solution to reach an end concentration, which was determined in 7.6.13
7.7.5 Invert, vortex and/or sonicate the solution (7.7.4) until the hemoglobin is fully solved.
7.7.6 Dispense 100 μl in glass tubes.
7.7.7 Dry the dispensed solution (7.7.6) in a rotating vacuum dryer (6.1) (heating point 1, 15 min., total run time: 60 min.)
7.7.8 Stopper the tubes and store at 4-7° C. until further use.

Typical baseline responses of S goldcoast LPS-hemoglobin complexes immobilized CM5 chip are given in FIG. 43.

### 8. Tables

TABLE 42
Addition of hemoglobin (5.7.3), sodium acetate buffer (5.7.2) and mQ
(5.1.3) to LPS (7.2.1) prior to oxidation.

| | Percentage hemoglobin | | | | | | | | | |
|---|---|---|---|---|---|---|---|---|---|---|
| | 0% | 10% | 20% | 30% | 40% | 50% | 60% | 70% | 80% | 90% |
| LPS (0) | 100 μl | 100 μl | 100 μl | 100 μl | 100 μl | 100 μl | 100 μl | 100 μl | 100 μl | 100 μl |
| NaAc, 1M pH | 50 μl | 50 μl | 50 μl | 50 μl | 50 μl | 50 μl | 50 μl | 50 μl | 50 μl | 50 μl |
| 5.5 (0) | | | | | | | | | | |
| Hb (5 mg/ml) | 0 μl | 10 μl | 20 μl | 30 μl | 40 μl | 50 μl | 60 μl | 70 μl | 80 μl | 90 μl |
| (0) | | | | | | | | | | |
| mQ (0) | 350 μl | 340 μl | 330 μl | 320 μl | 310 μl | 300 μl | 290 μl | 280 μl | 270 μl | 260 μl |

TABLE 43
Relative hemoglobin starting amounts added to LPS.

| | |
|---|---|
| PS providing | |
| Salmonella strain | hemoglobin |

| | | | | |
|---|---|---|---|---|
| S. enteritidis (0) | 0% | 10% | 20% | 30% |
| S. goldcoast (0) | 20% | 30% | 40% | 50% |
| S. livingstone (0) | 20% | 30% | 40% | 50% |
| S. meleogridis (0) | 20% | 30% | 40% | 50% |
| S. typhimurium (0) | 0% | 10% | 20% | 30% |

TABLE 44
Expected results of (diluted) agglutination serum binding to immobilized Salmonella LPS

| | Agglutination sera | | | | | | | | | |
|---|---|---|---|---|---|---|---|---|---|---|
| | | | | | | | | | | O |
| LPS | | O5 | | | | | | | | poly |
| providing | O4 | 1:20 | O6, 7 | O8 | O9 | O10 | O12 | O19 | O poly | E |
| Salmonella | 1:20 | 0 | 1:20 | 1:20 | 1:20 | 1:20 | 1:20 | 1:20 | A-S 1:20 | 1:100 |
| strain | (5.2.1) | (5.2.2) | (5.2.3) | (5.2.4) | (5.2.5) | (5.2.6) | (5.2.7) | (5.2.8) | (5.2.9) | (5.2.10) |
| S. enteritidis (5.6.1) | − | − | − | − | 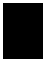 | − | 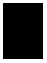 | − | 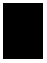 | − |
| S. goldcoast (5.6.2) | − | − | 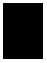 | 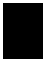 | − | − | − | − | 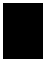 | − |
| S. livingstone (5.6.3) | − | − | 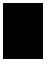 | − | − | − | − | − | 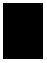 | − |
| S. melaegridis (5.6.4) | − | − | − | − | − | 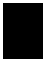 | − | − | 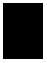 | 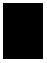 |
| S. typhimurium (5.6.5) | 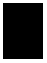 | 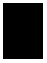 | − | − | − | − | 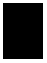 | − | 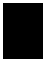 | − |

Legend:
+ = positive binding of serum to immobilized LPS
− = no binding of serum to immobilized LPS

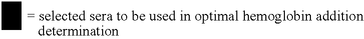

TABLE 45
Expected results of (diluted) avian reference serum binding to
immobilized Salmonella LPS

| | Avian reference sera | | | | |
|---|---|---|---|---|---|
| LPS providing | CH-SPF | EIA-St | EIA-Se | Spg | Si |
| Salmonella | 1:20 | 1:20 | 1:200 | 1:200 | 1:200 |
| strain | (5.4.1) | (5.4.3) | (5.4.2) | (5.4.4) | (5.4.5) |
| S. enteritidis (5.6.1) | 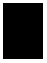 | + | 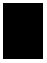 | 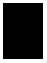 | − |
| S. goldcoast (5.6.2) | 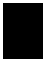 | − | − | − | + |
| S. livingstone (5.6.3) | 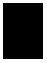 | − | − | − | 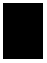 |
| S. melaegridis (5.6.4) | 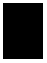 | − | − | − | − |
| S. typhimurium (5.6.5) | 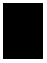 | 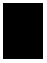 | + | + | − |

Legend:
+ = positive binding of serum to immobilized LPS
− = no binding of serum to immobilized LPS

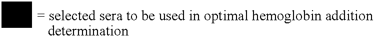

TABLE 46
Expected results of (diluted) swine reference serum binding to
immobilized Salmonella LPS

| | Avian reference sera | | |
|---|---|---|---|
| LPS providing | Sw-Liv | Sw-Typ | Sw-APP |
| Salmonella strain | (1:20) (5.5.1) | 1:20 (5.5.2) | 1:20 (5.5.3) |
| S. enteritidis (5.6.1) | − | + | − |
| S. goldcoast (5.6.2) | 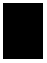 | − | − |
| S. livingstone (5.6.3) | 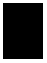 | − | − |
| S. melaegridis (5.6.4) | − | − | − |
| S. typhimurium (5.6.5) | − | 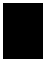 | − |

Legend:
+ = positive binding of serum to immobilized LPS
− = no binding of serum to immobilized LPS

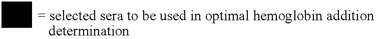

TABLE 47
Typical serological responses on Salmonella goldcoast immobilized LPS
with variable hemoglobin additions (data of two prepared CM5 chips)

| | | | | | Anti-Salm | | |
|---|---|---|---|---|---|---|---|
| | | | | O poly A-S | Group C | CH-SPF | Sw-Liv |
| Hemoglobin | Immobilization | O6, 7 1:20 | O8 1:20 | 1:20 | (1:100) | 1:20 | (1:20) |
| (%) | levels (RU) | (5.2.3) | (5.2.4) | (5.2.9) | (5.3.2) | (5.4.1) | (5.5.1) |
| 30 | 4596 | 273.2 | 257.1 | 66.8 | 76.8 | −9.0 | 53.7 |
| 40 | 4837 | 262.4 | 238.3 | 65.0 | 67.1 | −9.1 | 51.9 |
| 50 | 6112 | 271.9 | 237.5 | 60.9 | 71.6 | −13.1 | 47.1 |
| 60 | 9764 | 221.3 | 177.8 | 14.2 | 41.5 | −52.0 | 10.6 |
| 10 | 2177 | 179.4 | 140.0 | 39.0 | 55.0 | 0.5 | 28.0 |
| 20 | 3469 | 186.5 | 143.4 | 37.4 | 55.1 | −5.3 | 26.5 |
| 30 | 3690 | 228.3 | 186.0 | 50.1 | 80.1 | −4.7 | 35.1 |
| 40 | 5922 | 247.5 | 198.3 | 49.7 | 110.3 | −11.2 | 40.6 |

TABLE 48
Typical table of subtraction of CH-SPF from the selected positive
serological responses (data of Table 47) on Salmonella goldcoast immobilized LPS
with variable hemoglobin additions (data of two prepared CM5 chips).

| | | | | | Anti-Salm | |
|---|---|---|---|---|---|---|
| | | | | O poly A-S | Group C | Sw-Liv |
| Hemoglobin | Immobilization | O6, 7 1:20 | O8 1:20 | 1:20 | (1:100) | (1:20) |
| (%) | levels (RU) | (5.2.3) | (5.2.4) | (5.2.9) | (5.3.2) | (5.5.1) |
| 30 | 4596 | 282.2 | 266.0 | 75.8 | 85.7 | 62.6 |
| 40 | 4837 | 271.6 | 247.4 | 74.1 | 76.2 | 61.0 |
| 50 | 6112 | 284.9 | 250.6 | 74.0 | 84.6 | 60.2 |
| 60 | 9764 | 273.3 | 229.8 | 66.2 | 93.5 | 62.6 |
| 10 | 2177 | 178.9 | 139.5 | 38.5 | 54.6 | 27.5 |
| 20 | 3469 | 191.8 | 148.7 | 42.7 | 60.4 | 31.8 |
| 30 | 3690 | 233.0 | 190.7 | 54.8 | 84.8 | 39.8 |
| 40 | 5922 | 258.7 | 209.5 | 60.9 | 121.5 | 51.8 |

TABLE 49
Typical table of relative values score of Table 48 (data of two prepared
CM5 chips).

| | | | | | Anti- | | |
|---|---|---|---|---|---|---|---|
| | | | | | Salm | | |
| | | O6, 7 | | O poly A-S | Group C | Sw-Liv | |
| Hemoglobin | Immobilization | 1:20 | O8 1:20 | 1:20 | (1:100) | (1:20) | |
| (%) | levels (RU) | (5.3.2) | (5.2.4) | (5.2.9) | (5.3.2) | (5.5.1) | sum |
| 30 | 4596 | 10 | 10 | 10 | 9 | 10 | 49 |
| 40 | 4837 | 10 | 9 | 10 | 8 | 10 | 47 |
| 50 | 6112 | 10 | 9 | 10 | 9 | 10 | 48 |
| 60 | 9764 | 10 | 9 | 9 | 10 | 10 | 47 |
| 10 | 2177 | 7 | 7 | 6 | 4 | 5 | 30 |
| 20 | 3469 | 7 | 7 | 7 | 5 | 6 | 33 |
| 30 | 3690 | 9 | 9 | 9 | 7 | 8 | 42 |
| 40 | 5922 | 10 | 10 | 10 | 10 | 10 | 50 |

### 9. Formulas

### 9.1 Formulae 1

Calculation of Volume of mQ

v=w/5
v=volume of mQ (5.1.3) (in ml)
w=weight of LPS (7.1.2) (mg) (see SOP ChemieA21 (Example 11))

### 9.2 Formulae 2

Calculation of Relative Value of Positive Sera

Rv=Mv/Mhv*10
Rv=relative value
Mv=mean value (7.6.1)
Mhv=mean highest value (7.6.8)

### 9.3 Formulae 3

### Calculation of Amount Remaining LPS

z=w*0.005
z=amount of remaining LPS (in mg)
w=weight of remaining LPS solution (7.7.1) (in mg)

### 9.4 Formulae 4

### Calculation of Amount of Hemoglobin

h=y*z*0.01
h=mass of hemoglobin (in mg)
y=optimal hemoglobin percentage (%) (7.6.13)
z=amount of remaining LPS (9.3) (mg)

### Example 14

### Polysaccharides (PS) Isolated from LPS Coupled to Microspheres to Test Anti-Salmonella Serum Antibodies

### Materials and Methods:

Batches of LPS were obtained as described in Example 11 (“Extraction of lipopolysaccharides from salmonella spp.”).
mild acid hydrolysis:
   Stock solutions:
   2% (v/v) acetic acid
   Milli Q quality water
   LPS solutions of S.t., S.e., S.g., S.m., S.l:
   S.t. 2005.1, not oxidated, without Hb, 5 mg/ml
   S.l. 2005.1, not oxidated, without Hb, 5 mg/ml
   S.g., not oxidated, without Hb, 5 mg/ml
   S.e. 2005.1, not oxidated, without Hb, 5 mg/ml
   S.m. 15-01-2007S.Bokn/BG, batch 8-01-03, 5 mg/ml

### Procedure of Mild Hydrolysis:

1. Pipette 1500 μl 5 mg/ml LPS solutions into 4-ml glass tube.
2. Add 1500 μl 2% (v/v) acetic acid stock solution.
3. Make a small punch hole in the glass tube's cap
4. Keep at 100° C. in a heating block for 3 h.

### 5. Chill on ice.

### 6. Centrifuge 10 min at 14,000 g.

7. Weigh 2-ml eppendorf vials, on an analytical balance.
8. Transfer the supernatant into eppendorf vials with known weight.
9. Determine weight on an analytical balance.

### 10. Centrifuge 10 min at 14,000 g.

11. Transfer the supernatant into fresh eppendorf vial with known weight.
12. Weigh the remaining eppendorf vial with the pellet (lipid A).
13. Lyophilize lipid A and PS in either vial for 48 h (or until dryness is obtained).
14. Following lyophilisation, weigh eppendorf vials again and determine lipid A and PS dry weights.
15. Dissolve PS in water to a final concentration of 5 mg/ml.

### Oxidation

Oxidize PS according to the protocol given in Example 9 (“Immobilisation of salmonella-derived LPS onto fluorescent beads and detection of antibodies reporting a current or past salmonella infection in various biological samples”).

### Coupling of Oxidized Polysaccharides to Microspheres

Coupling of oxidized PS was performed according to the protocol given in Example 9 (“Immobilisation of salmonella-derived LPS onto fluorescent beads and detection of antibodies reporting a current or past salmonella infection in various biological samples”).

### Results

Negative and positive porcine sera were used to assess the
activity of the microspheres coated with either the usual
lipopolysaccharide (LPS) or isolated polysaccharide (PS).

| | Pig 1 | Pig 3 | Pig 4 | Pig 5 | Pig 6 | Pig 7 | Pig 8 | Pig 9 | Pig 1 [symbol] |
|---|---|---|---|---|---|---|---|---|---|
| | S/N (average to DL), B group | | | | | | | | |
| LPS | 2.97 | 0.26 | 0.47 | 0.42 | 0.86 | 0.42 | 2.64 | 0.53 | 0.35 |
| PS | 0.49 | 0.29 | 0.32 | 0.31 | 0.45 | 0.28 | 0.35 | 0.49 | 0.20 |
| | S/N (average to DL), C1 group | | | | | | | | |
| LPS | 0.30 | 8.46 | 0.38 | 0.80 | 0.39 | 0.21 | 0.16 | 7.20 | 0.62 |
| PS | 0.27 | 1.54 | 0.13 | 0.25 | 0.19 | 0.14 | 0.11 | 1.10 | 0.23 |
| | S/N (average to DL), C2 group | | | | | | | | |
| LPS | 0.68 | 0.59 | 3.85 | 0.44 | 0.47 | 0.17 | 0.59 | 0.67 | 6.65 |
| PS | 0.44 | 0.32 | 0.74 | 0.33 | 0.28 | 0.18 | 0.16 | 0.31 | 1.13 |
| | S/N (average to DL), D group | | | | | | | | |
| LPS | 3.33 | 0.30 | 0.17 | 5.17 | 0.24 | 0.60 | 1.18 | 0.75 | 0.18 |
| PS | 0.63 | 0.30 | 0.26 | 0.80 | 0.46 | 0.39 | 0.29 | 0.68 | 0.19 |
| | S/N (average to DL), E group | | | | | | | | |
| LPS | 0.53 | 0.39 | 0.25 | 0.53 | 6.28 | 0.60 | 0.35 | 0.38 | 0.40 |
| PS | 0.47 | 0.42 | 0.29 | 0.42 | 1.10 | 0.38 | 0.27 | 0.46 | 0.28 |

[symbol]

indicates data missing or illegible when filed

Positive sera for which binding was expected, are underlined. Reference sera from immunized animals were reactive as follows:

Pigs 1 and 8: Salmonella serogroup B
Pigs 3 and 9: Salmonella serogroup C₁
Pigs 4 and 10: Salmonella serogroup C₂
Pigs 5 and 11: Salmonella serogroup D
Pigs 6 and 12: Salmonella serogroup E
Pig 7: negative reference

Results were expressed as a signal to noise ratio, in which the noise was defined as the average response from negative sera plus three times the standard deviation. The results in the Table show improved specific responses of reference sera with coupled LPS compared to coupled PS.

### Example 15

### Coupling of LPS Using DMTMM

Three procedures for this alternative coupling of LPS using 4-(4,6-dimethoxy[1,3,5]triazin-2-yl)-4-methyl-morpholinium (DMTMM) were followed. These three procedures are listed here below:

### Procedure 1 (Using COOH-Beads)

### COOH-DMTMM:

1. Dissolve 2.5 mg of each LPS serotype B, C1, C2, D or E in 2.5 mL water.
2. Add 200 μL 200 mg/mL DMTMM in water.
3. Incubate 1 h on a gyrorocker at ambient temperature.
4. Equilibrate Sephadex G-25M PD10 columns with 5 mL PBS.
5. Transfer modified LPS to the PD10 column.
6. Elute LPS with 3:5 mL PBS.
7. Pipette 100 μL microspheres (BioPlex COOH-beads, 1.25×10⁶ beads/mL) in a vial (treated to prevent static electricity).
8. Spin beads down for 5 min at 13,000 g.
9. Add 50 μL DMTMM modified LPS to the microspheres and vortex-mix.
10. Incubate overnight on a Gyrorocker protected from light and at ambient temperature.
11. Centrifuge suspension at 13,000 g for 5 min.
12. Discard supernatant, add 100 μL PBS, repeat centrifugation and remove liquid.
13. Store the microspheres in 100 μL PBS (pH 7.2) containing 1% (v/v) equine serum and 0.01% (m/v) Proclin 150.

### Procedure 2 (Using Hydrazide-Containing Beads)

As an alternative, microspheres with a surface containing hydrazide functions can be used to couple LPS covalently to the surface using DMTMM. In this case, the original COOH surface has to be modified using EDC/NHS and carbohydrazide. Following the succinimide conjugation through the EDC/NHS combination, carbohydrazide is coupled to the microsphere, which is then the target for attachment of DMTMM-modified LPS.
1. Dissolve 2.5 mg of each LPS serotype B, C1, C2, D or E in 2.5 mL water.
2. Add 200 μL 200 mg/mL DMTMM in water.
3. Incubate 1 h on a gyrorocker at ambient temperature.
4. Equilibrate Sephadex G-25M PD10 columns with 5 mL PBS.
5. Transfer modified LPS to the column.
6. Elute LPS with 3.5 mL PBS.
7. Pipet 100 μL microspheres (BioPlex COOH-beads, 1.25×10⁶ beads/mL) in a vial (treated to prevent static electricity).
8. Spin beads down for 5 min at 13,000 g.
9. Remove supernatant and vortex-mix.
10. Add 100 μL EDC/NHS solution (Biacore) and incubate 20 min.
11. Spin particles down at 13,000 g for 5 min
12. Remove supernatant and vortex left suspension.
13. Add 100 μL, carbohydrazide and vortex-mix.
14. Spin beads down at 13,000 g for 5 min, remove supernatant and vortex-mix remaining suspension.
15. Add 5.0 μL DMTMM-modified LPS to activated microspheres and vortex-mix.
16. Incubate overnight on a gyrorocker protected from light and at ambient temperature.
17. Centrifuge suspension at 13,000 g for 5 min.
18. Discard supernatant, add 100 μL PBS, repeat centrifugation and remove liquid.
19. Store the microspheres in 100 μL PBS (pH 7.2) containing 1% (v/v) equine serum and 0.01% (m/v) Proclin 150.

### Procedure 3 (Using Hydrazide-Containing Beads and Ethanolamine)

In order to prevent the involvement of non-used (re)active succinimide sites on the microsphere in serum diagnostics, ethanolamine was added after carbohydrazide incubation and before addition of DMTMM-modified LPS.
1. Dissolve 2.5 mg of each LPS serotype B, C1, C2, D or E in 2.5 mL water.
2. Add 200 μL 200 mg/mL DMTMM in water.
3. Incubate 1 h on a gyrorocker at ambient temperature.
4. Equilibrate Sephadex G-25M PD10 columns with 5 mL PBS.
5. Transfer modified LPS to the column.
6. Elute LPS with 3.5 mL PBS.
7. Pipet 100 μL microspheres (BioPlex COOH-beads, 1.25×10⁶ beads/mL) in a vial (treated to prevent static electricity).
8. Spin beads down for 5 min at 13,000 g.
9. Add 100 μL EDC/NHS solution (Biacore) and incubate 20 min.
10. Spin down at 13,000 g for 5 min, remove supernatant
11. Vortex-mix remaining suspension.
12. Add 100 μL carbohydrazide and spin beads down at 13,000 g for 5 min.
13. Remove supernatant and vortex-mix remaining suspension.
14. Add 100 μL ethanolamine
15. After 20 min incubation, spin beads down at 13,000 g for 5 min.
16. Remove supernatant and vortex.
17. Wash pelleted beads with 100 μL PBS and spin beads down at 13,000 g for 5 min.
18. Remove supernatant and vortex-mix.
19. Add 50 μL DMTMM-modified LPS to activated microspheres.
20. Incubate overnight.
21. Centrifuge at 13,000 g for 5 min, remove supernatant and vortex-mix.
22. Add 100 μL sodium cyanoborohydrid
23. Incubate for 60 min.
24. Centrifuge suspension at 13,000 g for 5 min.
25. Discard supernatant, add 100 μL PBS, repeat centrifugation and remove liquid.
26. Store the microspheres in 100 μL PBS (pH 7.2) containing 1% (v/v) equine serum and 0.01% (m/v) Proclin 150.

### Results

The responses of reference sera towards alternatively coupled LPS to either carboxylic (C/beads) or amino (A/beads) microspheres are summarized in FIG. 47.

Negative and positive sera were used to assess the activity of the carboxylic and amino microspheres coated with DMTMM-modified LPS (DMTMM/LPS). Reference sera used for this purpose were from immunized animals and were reactive as follows:

Animal 1 and 8: Salmonella serogroup B;
Animal 3 and 9: Salmonella serogroup C₁;
Animal 4 and 10: Salmonella serogroup C₂;
Animal 5 and 11: Salmonella serogroup D;
Animal 6 and 12: Salmonella serogroup E;
Animal 7: negative reference.

Results were expressed as a signal to noise ratio, in which the noise was defined as the average response from negative sera plus three times the standard deviation. The results of the DMTMM/LPS-C/beads show improved specific responses for C₁ and C₂ compared to the ‘default’ coupling, which is the method as described herein in all the other examples. The responses on the other serogroups are of minor quality. In case of DMTMM/LPS-A/beads, improved specific responses for C₁ and C₂ and a comparable result for serogroup E were observed compared to the ‘default’ coupling. When DMTMM/LPS-A/beads were prepared in the presence of ethanolamine, the result was improved over that without the ethanolamine addition. The DMTMM modification, however, dramatically affects (the antigenic structures) of serogroups B and D, which are considered the most important serogroups in Salmonella serology. These serogroups are most important as of all food-borne Salmonella infections, involved Salmonella serovars belong for the greatest part to these serogroups B and D.

It is clear from these results that a method according to the invention is particularly useful for obtaining a robust and/or sensitive carrier comprising serogroup B and/or D antigens.

### Example 16

### Coupling of LPS to Amino-Containing Microspheres

While investigating the suitability of alternative microspheres, i.e. from different suppliers it was observed that carboxylic microspheres from Duke Scientific Corporation were difficult to operate when testing porcine sera. As an alternative for the carboxy acid-containing microspheres, amino-containing particles can be used applying almost identical chemistry as described herein in examples 1-14, i.e. LPS oxidized in the presence of protein.

### Procedure:

One of the differences is that LPS is 8 fold more diluted, as described in examples 1-14. The final concentration of oxidized, protein-fortified LPS is 0.06 mg/mL. For this purpose, stocks of modified LPS were diluted in 10 mM NaAc (pH 4.0).
1. Transfer 200 μL 7.3×10⁷ microspheres in an anti-static treated vial (1.5 mL; anti-static treated vials).
2. Pellet microspheres at 13,000 g for 5 min.
3. Remove supernatant and leave approximately 10 μL liquid.
4. Vortex-mix suspension.
5. Add 200 μL 0.06 mg/mL LPS in 10 mM NaAc (pH=4.0)
6. Incubate while attached to a gyrorocker for 1.5 h.
7. Centrifugate suspension at 13,000 g for 5 min.
8. Remove supernatant and leave approximately 10 μL liquid.
9. Vortex-mix remaining particulate material.
10. Add 200 μL 100 mM sodium cyanoborohydride in 10 mM NaAc (pH 4.0).
11. Incubate mixture on a gyrorocker for 1 h.
12. Spin beads down at 13,000 g for 5 min.
13. Remove supernatant and leave approx. 10 μL
14. Vortex-mix remaining material.
15. Add 200 μL PBS (pH 7.2).
16. Centrifugate suspension at 13,000 g for 5 min.
17. Remove supernatant and leave approximately 10 μL liquid.
18. Vortex-mix the pelleted material.
19. Add and store in 200 μL PBS (pH 7.2) containing 1% (v/v) equine serum and 0.01% (m/v) Proclin 150.
20. Vortex-mix vigorously and store at 4° C. protected from light.

In FIG. 48 the responses of specific serum anti-Salmonella antibodies using COOH-containing or NH₂-containing beads are compared. This figure demonstrates the out-performance of the NH₂-beads over the COOH-beads in the case of porcine sera. In this Figure the key of identification is:

Animal 1 and 8: Salmonella Serogroup B immunized pigs;
Animal 3 and 9: Salmonella serogroup C₁ immunized pigs;
Animal 4 and 10: Salmonella serogroup C₂ immunized pigs;
Animal 5 and 11: Salmonella serogroup D immunized pigs;
Animal 6 and 12: Salmonella serogroup E immunized pigs;

The same kind of beads (i.e. LPS coupled to amino-containing microspheres) also provides good results in respect of chicken sera (not shown).

### DESCRIPTION OF FIGURES

FIG. 1. Schematic representation of one embodiment of the method according to the invention

FIG. 2. Schematic outline of the BIA for detection of bacteria using bacteriophages as indicator organisms. Indicator organisms may be cultured overnight or shorter.

FIG. 3. Reactivity of Hb-fortified, oxidized LPS isolated from S. typhimurium (batch St2003.2) with agglutination sera in relative arbitrary biosensor responses (RU). The Hb fortification level of LPS during oxidation is depicted in the figure. The expected binding of the agglutination sera is listed in Table 3.

FIG. 4. ROC curves from Example 2, Experiment 1. TPF: True-positive fraction; FPF: false-positive fraction.

FIG. 5. ROC curves from Example 2, Experiment 2. TPF: True-positive fraction; FPF: false-positive fraction.

FIG. 6. Analysis of prepared beads coated with LPS from S. enteritidis (reflecting serogroup D), S. goldcoast (reflecting serogroup C₂), S. livingstone (reflecting serogroup C₁), S. meleagridis (reflecting serogroup E) and S. typhimurium (reflecting serogroup B). The success of the coating and specificity of the LPS were tested with commercially available monoclonal antisera against O4 (serogroup B), O5 (serogroup B), O7 (serogroup C₁), O8 (serogroup C₂) and O9 (serogroup D). Responese are expressed in arbitrary units as median fluorescence index (MFI) at the Y-axis, whereas the X-axis indicates the type of LPS conjugation of the individual beads.

FIG. 7. Analysis of prepared beads coated with LPS reflecting serogroups B, C₁, C₂ and D. The activity of the coating was tested with monoclonal antisera against O4 (serogroup B), O5 (serogroup B), O7 (serogroup C₁), O8 (serogroup C₂) and O9 (serogroup D). Similar to FIG. 6, except zoomed in on the lower responses. Notice that response of anti-O5 is under broken. See for details legend of FIG. 6.

FIG. 8. Comparison of beads coated with two different oxidation batches of oxidized LPS from S. enteritidis (reflecting serogroup D) and S. goldcoast (reflecting serogroup C₂), S. livingstone (reflecting serogroup C₁) and S. typhimurium (reflecting serogroup B). The coating was tested with commercially available monoclonal antisera against O5 (serogroup B), O7 (serogroup C₁), O8 (serogroup C₂) and O9 (serogroup D).

FIG. 9. Analysis of meat drip and serum from chickens. Commercially available antisera were used to spike meat drip and serum. Drip, liquid extract collected from muscle tissue from a chicken, which was tested as Salmonella-free using standard ISO methods; Drip+CH-SPF, drip that was spiked with serum collected from specific pathogen free (SPF) chickens; CH-SPF, serum obtained from specific pathogen free (SPF) chickens; DripSPA-PG, drip that was spiked with antiserum reactive with S. pullorum and S. gallinarum; SPA-PG, anti-S. pullorum and anti-S. gallinarum antiserum; DripCHSi, drip that was spiked with chicken serum which was serologically positive for a S. infantis infection; CH-Si, chicken serum serologically positive for S. infantis. The X-axis indicate the type of LPS conjugation of the individual beads. See FIGS. 6, 7 and 8 for more details.

FIG. 10. Analysis of swine sera spiked with commercially available anti-S. typhimurium (yellow coloured bars) and anti-S. livingstone (cyan coloured bars). In addition, beads in buffer solution (blue coloured bars) and negative swine serum (purple coloured bars) were analysed on beads which were coated with LPS representing serogroups B, C₁, C₂ and D.

FIG. 11. Binding of bacteriophage FO1 to immobilized LPS from S. typhimurium, S. enteritidis, S. goldcoast and S. livingstone on a Biacore SPR biosensor. PFU, plaque forming units.

FIG. 12. Binding of bacteriophage FO1 to an SPR biosensor chip coated with S. typhimurium LPS following incubation of S. typhimurium, S. enteritidis, S. goldocoast, S. livingstone with 1.2×10⁹ PFU bacteriophage FO1. Dotted line indicates the cut off value.

FIG. 13. Incubation of different food pathogens and spoilage bacteria in the presence of Salmonella spp.-specific bacteriophage FO1. During growth the optical density at λ 600 nm as a measure of bacterial growth was monitored. bl-FO1, blank medium devoid of bacteria supplemented with bacteriophages exclusively.

FIG. 14. Incubation of different. Salmonella serovars in the presence of Salmonella spp.-specific bacteriophage FO1. See for more details legend of FIG. 13.

FIG. 15. Incubation of different food pathogens and spoilage bacteria in the presence of Salmonella spp.-specific bacteriophage FO1. The number of plaque forming units (PFU) was determined following an incubation of 5 h. bl+FO1, blank medium devoid of bacteria supplemented with bacteriophages exclusively.

FIG. 16. Incubation of different Salmonella serovars in the presence of Salmonella spp.-specific bacteriophage FO1. See for more details legend of FIG. 13. FO1 stock was not incubated.

FIG. 17. SPR biosensor analysis of bacteriophage FO1 propagated in different Salmonella serovars after concentration and dialysis of the viruses. The suspensions were serial diluted and analysed; the final concentrations of concentrated/diluted bacteriophages is indicated at the X-axis.

FIG. 18. Oxidation of carbohydrate moiety. R′ and R indicate the distal and the proximal positions, respectively, in the carbohydrate chain.

FIG. 19. Conjugation to a polyamine containing molecule (R″), such as a protein.

FIG. 20. Immobilization to fluorescent beads and stabilization of chemical bonds.

FIG. 21. Schematic representation of the procedure of LPS coupling to beads and analysis of serum.

FIG. 22a. Examples of Campylobacter-infecting bacteriophages.

FIG. 22b. Examples of Listeria-infecting bacteriophages.

FIG. 22c. Examples of Salmonella-infecting bacteriophages.

FIG. 23. Counting chamber and technique.

FIG. 24. Total area of a Bürker-Türk counting chamber (A) of 1 mm², in which B represents the area of 1/16^{th} of the total area.

FIG. 25. Effect of periodate concentration on the immobilization of LPS of S. enteritidis on a SPR biosensor chip.

FIG. 26. Effect of periodate concentration on the antigenic activity of immobilized LPS of S. enteritidis (batch Se2002.1). LPS was immobilized to a biosensor chip and analyzed in an SPR biosensor (FIG. 25). Range tested was 0.2 mM to 1.8 mM sodium periodate. O9, O12, O poly A-S, S. typh, SE, biosensor response from anti-O9 antisera; anti-O12 antisera, polyclonal antybody against serogroups A to S, chicken serum positive for S. typhimurium and chicken serum positive for S. enteridis, respectively.

FIG. 27. Effect of periodate concentration on the antigenic activity of immobilized LPS of S. enteritidis (batch Se2002.1). Range tested was 1.8 mM to 48.6 mM sodium periodate. See for more details FIG. 26.

FIG. 28. Effect of periodate concentration on the immobilization of LPS of S. goldcoast on a SPR biosensor chip.

FIG. 29. Effect of periodate concentration on the antigenic activity of immobilized LPS of S. goldcoast (batch Sg2002.1). LPS was immobilized to a biosensor chip and analyzed in an SPR biosensor (FIG. 28). Range tested was 0.2 mM to 5.4 mM sodium periodate. O6, O7, O8, O poly A-S, S. livingstone, S. infantis, biosensor response from anti-O6/7 antisera; anti-O8 antisera, polyclonal antybody against serogroups A to S, porcine serum positive for S. livingstone and chicken serum positive for S. infantis, respectively.

FIG. 30. Effect of periodate concentration on the immobilization of LPS of S. livingstone on a SPR biosensor chip.

FIG. 31. Effect of periodate concentration on the antigenic activity of immobilized LPS of S. goldcoast (batch Sg2002.1). LPS was immobilized to a biosensor chip and analyzed in an SPR biosensor (FIG. 30). Range tested was 0.2 mM to 5.4 mM sodium periodate. O6, 7, O8, O poly A-S, S. livingstone, S. infantis, biosensor response from anti-O6/7 antisera; anti-O8 antisera, polyclonal antybody against serogroups A to S, porcine serum positive for S. livingstone and chicken serum positive for S. infantis, respectively.

FIG. 32. Schematic presentation of the procedure.

FIG. 33. Quality sheet for LPS extraction process.

FIG. 34. Immobilization of LPS to biosensor surface and stabilization of chemical bonds.

FIG. 35. Biacore 3000 control software on COM 1.

FIG. 36. Immobilization wizard.

FIG. 37. Immobilization test wizard.

FIG. 38. Logging.

FIG. 39. Typical sensorgram of immobilization of oxidized LPS. Report points: 1 baseline; 2 activating EDC/NHS; 3 carbohydrazide; 4 ethanolamine; 5 Immobilization LPS Se.

FIG. 40. Sensorgram of anti Salmonella O Poly A-S analysed on a LPS-containing CM5 chip

FIG. 41. Typical serological responses of agglutination sera and group specific Salmonella anti sera on a S goldcoast LPS-hemoglobin immobilized CM5 biosensor chip.

FIG. 42. Typical serological responses of avian reference sera (SPF-CH, EIA St, EIA Se, SPA-PG and CH-Si sera) and swine reference sera (SW-sera) on a S. goldcoast LPS-hemoglobin immobilized CM5 biosensor chip.

FIG. 43. Typical baseline responses of a S goldcoast LPS-hemoglobin immobilized CM5 biosensor chip.

FIG. 44. ImmuSpeed™ analysis of meat drip, which was derived from an experiment in which chickens were experimentally infected with Salmonella enteritidis. Negative samples were from control chickens that were not infected. Meat drip samples were diluted 1:100 (v/v) in Tris 100 mM at pH 7. The secondary antibody was donkey-anti-chicken, diluted 1:150 (v/v) in Tris 100 mM pH 7 containing 1% (v/v) FCS.

FIG. 45 Repetitive Octet™ analysis of Salmonella negative SPF Chicken serum (yellow line) and of chiken serum positive for S. pullorum-galinarum. Time in seconds of the first analysis cycle is indicated. Steps in analysis are 1) contacting PBS pH 7 (baseline), 2) sampling serum, 3) regeneration. A cycle starts with PBS pH 7 again.

FIG. 46 Octet™ analysis of anti-O5 MAB on three biosensors (green, red and purple curves), which were all coated with serogroup B antigen. Negative chicken serum was tested on two other biosensor surfaces (yellow an light purple curves), while one biosensor was not coated with LPS at all (blue line) and one biosensor was not contacted with liquid. At time is 600 s, biosensors were submerged in samples and replaced after 180 s.

FIG. 47 DMTMM-modified LPS coupled to carboxylic beads (purple bars; first in each set), to amino-beads (wine-red bars; second in each set), to amino-beads treated with ethanolamine (yellow bars; third in each set) and to carboxylic beads using coupling chemistry described herein (‘Default’, cyan colored bars; last column in each set)

Norm_MFI/DL=normalized mean fluorescence intensity/decision limit

FIG. 48 Signal to noise (S/N) ratios of sera from pigs immunized with specific Salmonella serovars. S/N ratios of COOH-beads are indicated in the blue coloured bars (left bar in each set), whereas those of the NH2-beads are indicated with the wine-red coloured bars (right bar in each set).

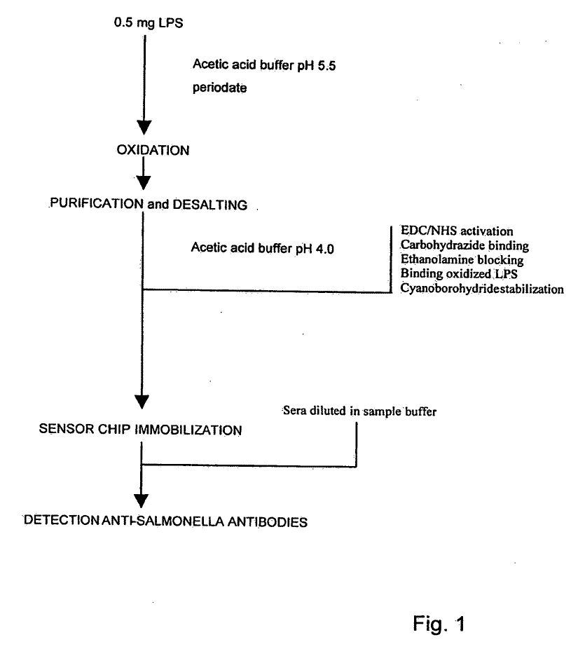

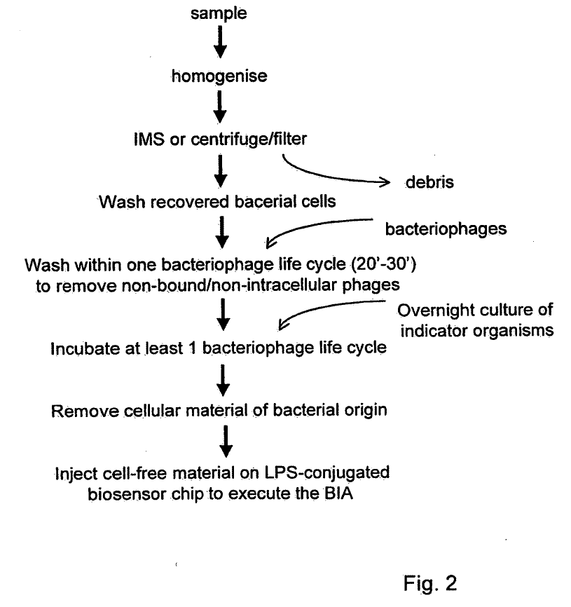

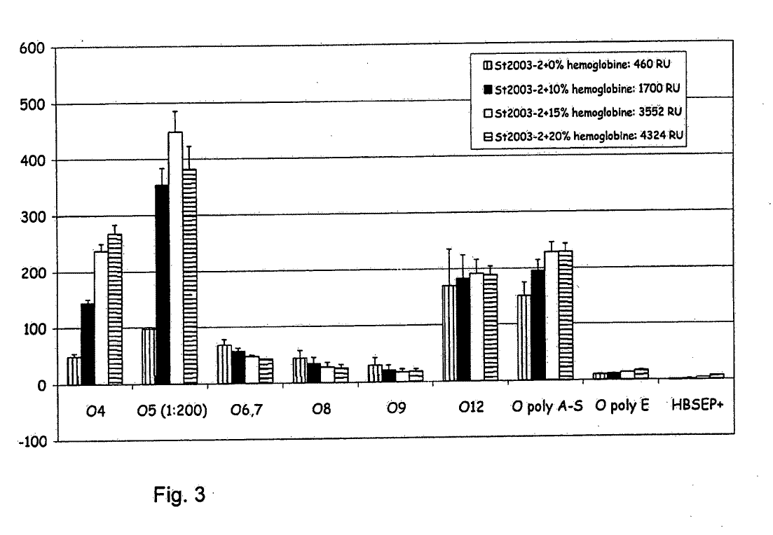

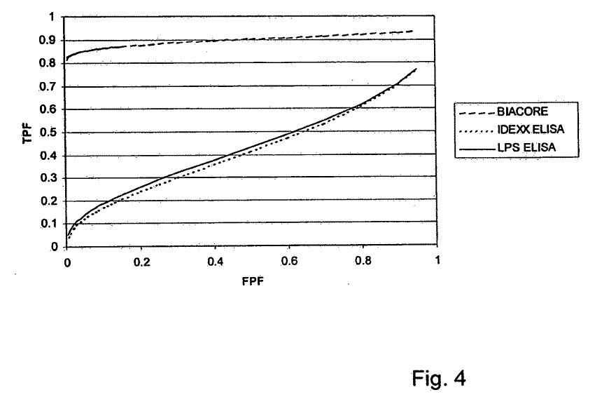

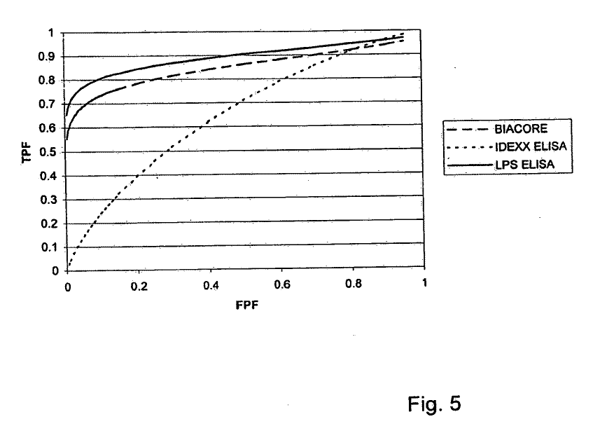

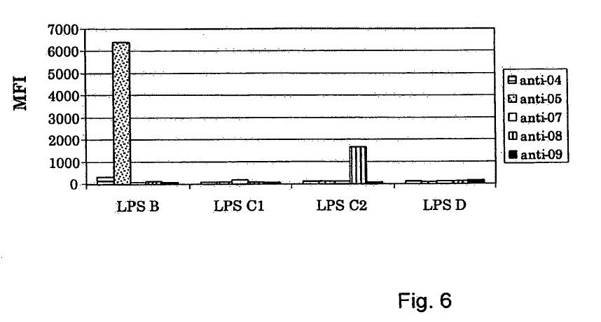

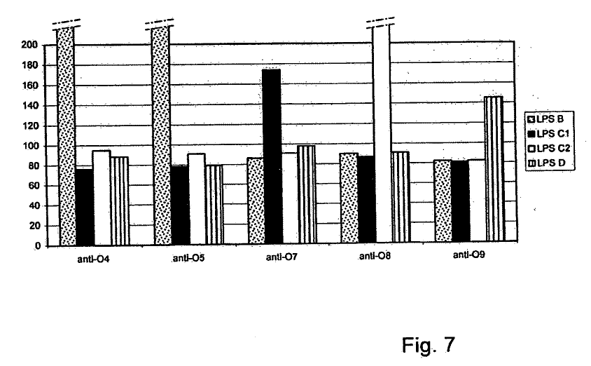

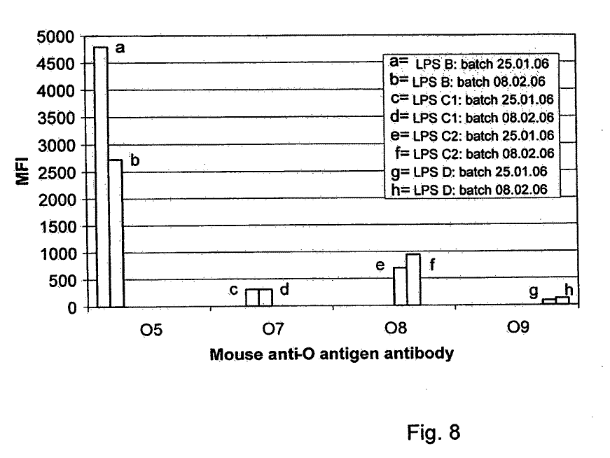

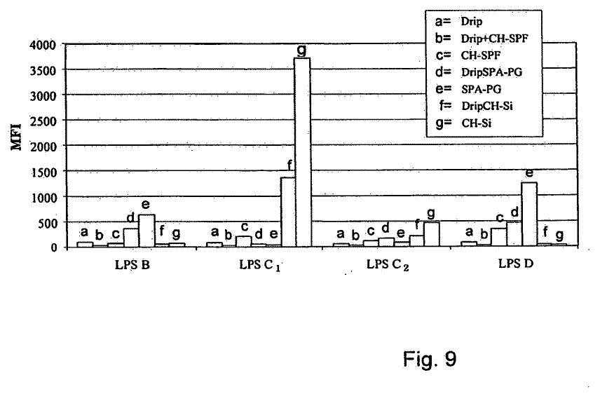

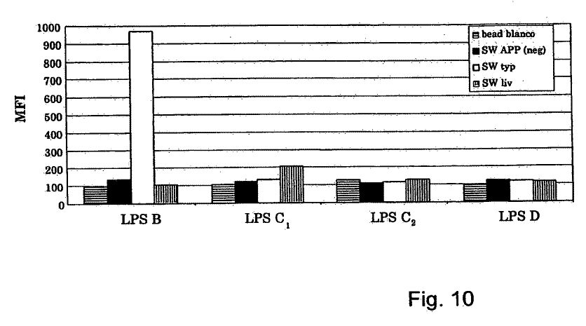

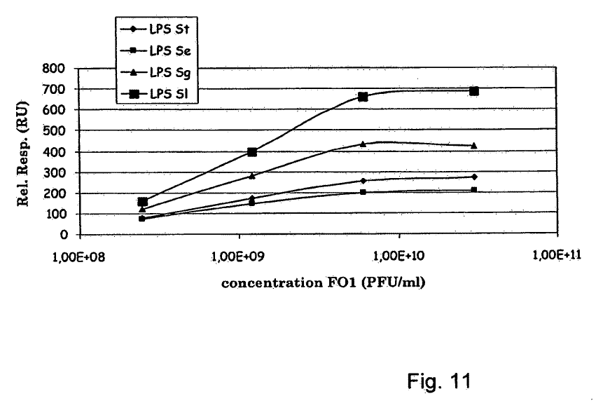

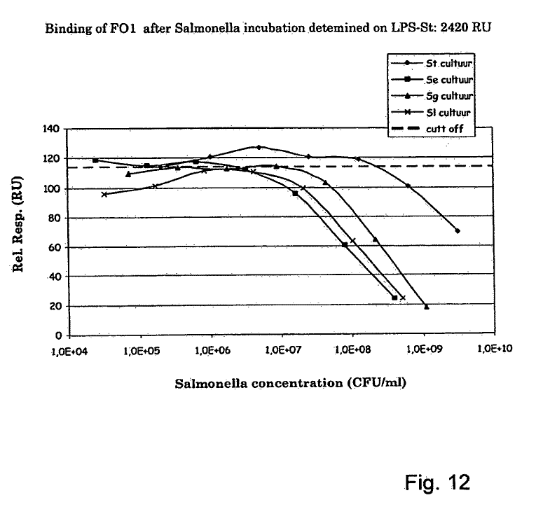

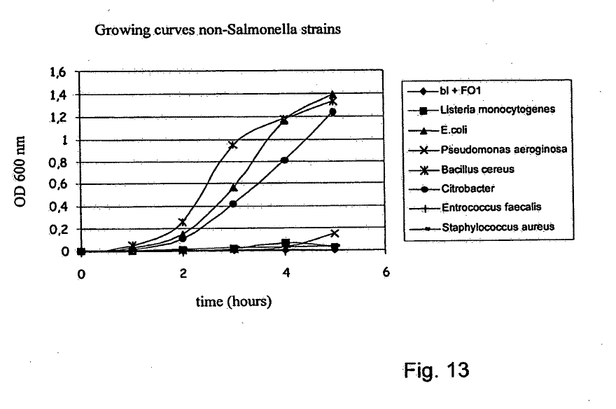

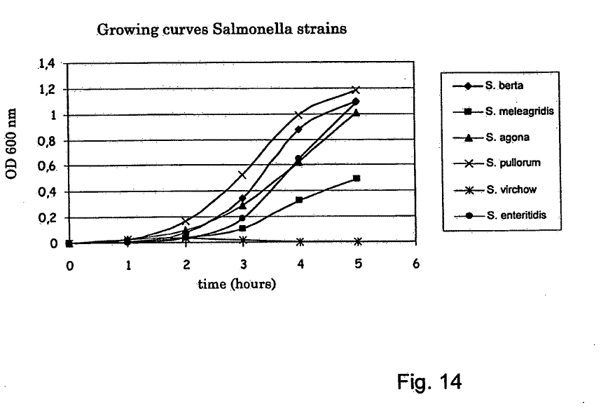

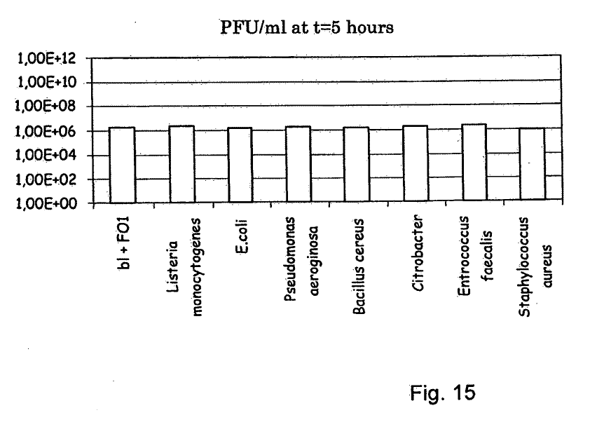

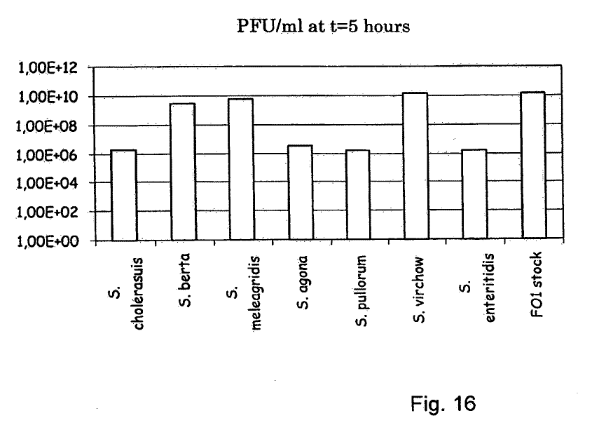

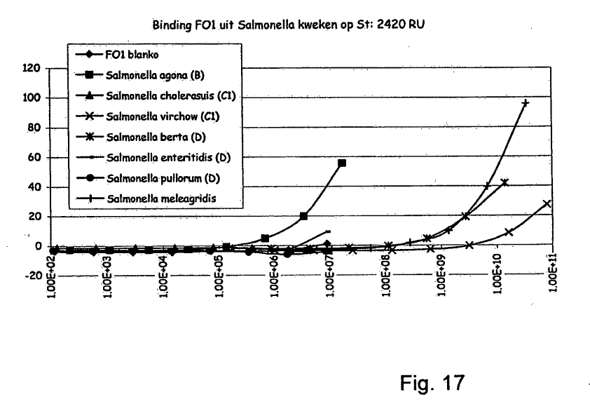

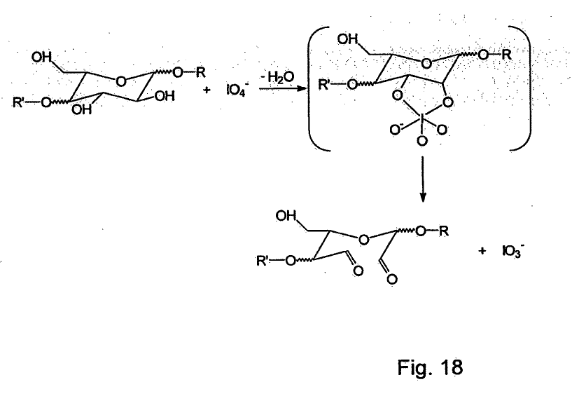

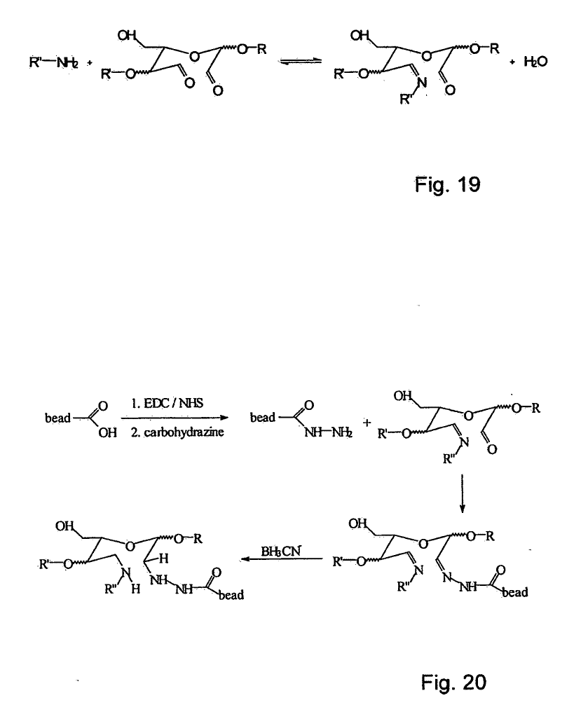

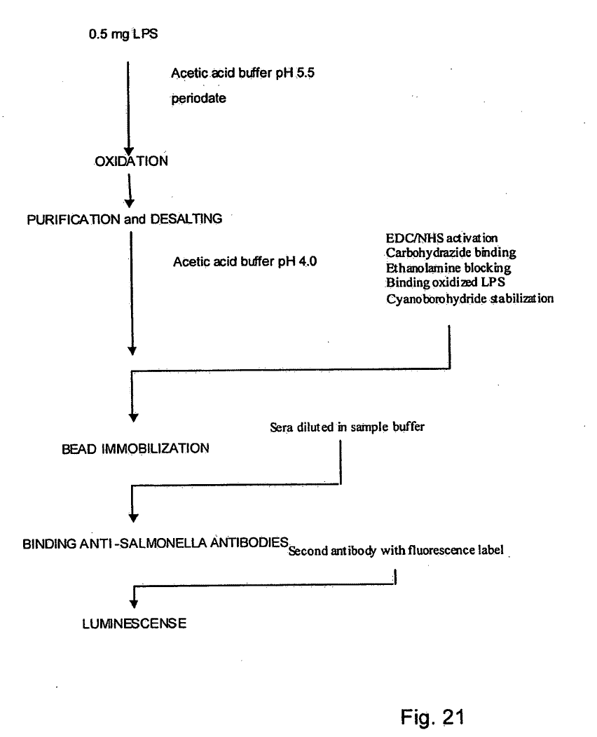

Norm_MFI/DL=normalized mean fluorescence intensity/decision limit

### REFERENCES

Barrow, P., 2000. Serological diagnosis of Salmonella by ELISA and other tests. In: Wray, C., Wray, A. (Eds.), Salmonella in Domestic. Animals. CAB International, Oxon, p. 407.
Barrow, P A, Desmidt, M., Ducatelle, R., Guittet, M., van der Heijden, H. M., Holt, P. S., Huis in't Velt, J. H., McDonough, P., Nagaraja, K. V., Porter, R. E., Proux, K., Sisak, F., Staak, C., Steinbach, G., Thorns, C. J., Wray, C., van Zijderveld, F., 1996. World Health Organisation-supervised interlaboratory comparison of ELISAs for the serological detection of Salmonella enterica serotype enteritidis in chickens. Epidemiol. Infect. 117, 69.
Berends, B. R., van Knapen, F., Mossel, D. A., Burt, S. A., Snijders, J. M., 1998. Impact on human health of Salmonella spp. on pork in The Netherlands and the anticipated effects of some currently proposed control strategies. Int. J. Food Microbiol. 44, 219.
de Vries, N., Zwaagstra, K. A., Huis in't Veld, J. H., van Knapen, F., van Zijderveld, F. G., Kusters, J. G., 1998. Production of monoclonal antibodies specific for the i and 1,2 flagellar antigens of Salmonella typhimurium and characterization of their respective epitopes. Appl. Environ. Microbiol. 64, 5033.
Edel, W., van Leeuwen, W. J., Hoogenboom Verdegaal, A. M., 1993. The annual incidence of salmonellosis in humans in The Netherlands. Tijdschr. Diergeneeskd. 118, 306.
Fratamico, P. M., Strobaugh, T. P., Medina, M. B., Gehring, A. G., 1997. Real-time detection of Escherichia coli O157:H7 using a surface plasmon resonance biosensor. Book of Abstracts, 214th ACS National Meeting, Las Vegas, Nev., September 7-11, AGFD.
Grimont, P., Grimont, F., Bouvet, P., 2000. Taxonomy of the genus Salmonella. In Wray, c., Wray, A. (Eds.), Salmonella in Domestic Animals. CAB International, Oxon, p. 1.
Holt, P., 2000. Host susceptibility, resistance and immunity to Salmonella in animals. In: Wray, C., Wray, A. (Eds.), Salmonella in Domestic Animals. CAB International, Oxon, p. 73.
Hoogenboom Verdegaal, A. M., de Jong, J. C., During, M., Hoogenveen, R., Hoekstra, J. A., 1994. Community-based study of the incidence of gastrointestinal diseases in The Netherlands. Epidemiol. Infect. 112, 481.
Ivnitski, D., Abdel-Hamid, I., Atanasov, P., Wilkins, E., 1999. Biosensors for detection of pathogenic bacteria. Biosens. Bioelectron. 14, 599.
Jongerius-Gortemaker, B. G. M., B. G., Goverde, R. L., van Knapen, F., Bergwerff, A. A., 2002. Surface plasmon resonance (BIACORE) detection of serum antibodies against Salmonella enteritidis and Salmonella typhimurium, J. Immunol. Meth. 266, 33-44
Kamerling J P, Vliegenthart J F G, Carbohydrates. In Clinical Biochemistry, Principles, Methods, Applications. Mass Spectrometry, edited by Lawson A M (Walter de Gruyter, Berlin, 1989), vol. 1, pp. 175-263.
Kretschmann, E., Raether, H., 1968. Radiative decay of nonradiative surface plasmons excited by light. Z. Naturforsch. A 23, 2135.
Liedberg, B., Nylander, C., Lundstroem, I., 1983. Surface plasmon resonance for gas detection and biosensing. Sens. Actuators 4, 299.
Medina, M. B., 1997. SPR biosensor: food science applications. Food Test. Anal. 3, 14.
Medina, M. B., Van Houten, L., Cooke, P. H., Tu, S. I., 1997. Realtime analysis of antibody binding interactions with immobilized E. coli O157:H7 cells using the BIAcore. Biotechnol. Technol. 11, 173.
Popoff, M. Y. (2001) In: Antigenic formulas of the Salmonella serovars, 8th revision. WHO Collaborating Centre for Reference and Research on Salmonella. Institut Pasteur, Paris, France, pp. 150.
Staub, A. M. (1965) Bacterial lipido-proteino-polysaccharides (‘O’ somatic antigens): extraction with trichloroacetic acid. In Whistler, R. L. (ed.), Methods in Carbohydrate Chemistry, Volume V. Academic. Press, New York, pp. 92-93
Thorns, C. J., Bell, M. M., Sojka, M. G., Nicholas, R. A., 1996. Development and application of enzyme-linked immunosorbent assay for specific detection of Salmonella enteritidis infections in chickens based on antibodies to SEF14 fimbrial antigen. J. Clin. Microbiol. 34, 792.
van Asten, A. J., Zwaagstra, K. A., Baay, M. F., Kusters, J. G., Huis in't Veld, J. H., van der Zeijst, B. A., 1995. Identification of the domain which determines the g,m serotype of the flagellin of Salmonella enteritidis. J. Bacteriol. 177, 1610.
Van Pelt, W., Van de Giessen, A., Van Leeuwen, W., Wannet, W., Henken, A., Evers, E., De Wit, M., Van Duynhoven, Y., 1999. Oorsprong, omvang en kosten van humane salmonellose. Deel 1. Oorsprong van humane salmonellose met betrekking tot varken, rung, kip, ei en overige bronnen. Infectieziekten Bull. 10, 240.
W. van Pelt, W. J. B. Wannet, A. W. van de Giessen, Y. T. H. P. van Duynhoven, 2003. Trends in gastroenteritis in the Netherlands Lowest incidences in 2002 ever; the lull before the storm? Infectieziekten bull. 14, 424.
Wilkons S. G., 1996, Bacterial lipopolysaccharides—Themes and variations. Progress in Lipid Research, volume 35, issue 3, September, p. 283-343
Yamane, Y., Awamura, N., Fujii, H., Ohta, H., Toyota, Y., Otsuki, K., Inoue, T., 2000. Establishment of an enzyme-linked immunosorbent assay with a coated deflagellated Salmonella enteritidis antigen for detection of a specific chicken antibody. Avian Dis. 44, 291.

### References Example 2

Baay, M. F., Huis in 't Veld, J. H. (1993) Alternative antigens reduce cross-reactions in an ELISA for the detection of Salmonella enteritidis in poultry. J. Appl. Bacteriol. 74, 243.
Bergwerff, A. A., van Knapen, F. (2006) Surface Plasmon Resonance Biosensors for Detection of Pathogenic Micro-organisms: Strategies to Secure Food and Environmental Safety. J. A.O.A.C. Int. 89, 826-831.
Bergwerff, A. A., van Knapen, F. (2003) Sensing pathogens: Screening strategies in food and environmental safety. Biacore Journal 2, 10-15.
Bokkers, E. G. M. (2002). The Action Plan Salmonella Poultry Layer Sector, 2001+. Dutch Product Board for Livestock, Meat and Eggs. Available, from http://bedrijfsnet.pve.agro.nl/pls/pbs/docs/folder/BEDRIJFSNET_US_CA/HOOFDTHEMAS/KWALITEIT_VOEDSELVEILIGHEID/SALMONELLA_CAMPYLOBACTER/ACTIEPLANNEN/EIERSECTOR/ARTMEL_ACTIEPLANSALMONELLA_%20EIERSECTOR2001.PDF. In Dutch. Last visited: December 2005
Desmidt, M., Ducatelle, R., Haesebrouck, F., de Groot, P. A., Verlinden, M., Wijffels, R., Hinton, M., Bale, J. A., Allen, V. M. (1996) Detection of antibodies to Salmonella enteritidis in sera and yolks from experimentally and naturally infected chickens. Vet. Rec. 138, 223-6.
Van Duijkeren E., Wannet, W. J., Houwers, D. J., van Pelt, W. (2002). Serotype and phage type distribution of salmonella strains isolated from humans, cattle, pigs, and chickens in the Netherlands from 1984 to 2001. J. Clin. Microbiol. 40, 3980-5.
Van Duynhoven, Y. T., de Jager, C. M., Kortbeek, L. M., Vennema, H., Koopmans, M. P., van Leusden, F., van der Poel, W. H., van den Broek, M. J. (2005) Explosie Project Team. A one-year intensified study of outbreaks of gastroenteritis in The Netherlands. Epidemiol. Infect. 133, 9-21.
Fischer, I. S. T. (2004) International trends in Salmonella serotypes 1998-2003—a surveillance report from the Enter-net international surveillance network. Euroroundup 9, 9-10.
Gast, R. K., Nasir, M. S., Jolley, M. E., Holt, P. S., Stone, H. D. (2002) Detection of experimental Salmonella enteritidis and S. typhimurium infections in laying hens by fluorescence polarization assay for egg yolk antibodies. Poult. Sci. 81, 1128-31.
Gast, R. K., Porter, R. E. Jr., Holt P. S. (1997) Applying tests for specific yolk antibodies to predict contamination by Salmonella enteritidis in eggs from experimentally infected laying hens. Avian Dis. 41, 195-202.
Gast, R. K., Beard, C. W. (1991) Detection of Salmonella serogroup D-specific antibodies in the yolks of eggs laid by hens infected with Salmonella enteritidis. Poult. Sci. 70, 1273-6.
Guerin, M. T., Martin, S. W., Darlington, G. A., Rajic, A. (2005). A temporal study of Salmonella serovars in animals in Alberta between 1990 and 2001. Can. J. Vet. Res. 69, 88-99.
Hassan, J. O., Barrow, P. A., Mockett, A. P., McLeod, S. (1990) Antibody response to experimental Salmonella typhimurium infection in chickens measured by ELISA. Vet. Rec. 126, 519.
Holt, P. S., Stone, H. D., Gast, R. K., Greene, C. R. (2000) Application of the agar gel precipitin test to detect antibodies to Salmonella enterica serovar enteritidis in serum and egg yolks from infected hens. Poult. Sci. 79, 1246-50.
Jongerius-Gortemaker, B. G., Goverde, R. L., van Knapen, F., Bergwerff, A. A. (2002) Surface plasmon resonance (biacore) detection of serum antibodies against Salmonella enteritidis and Salmonella typhimurium. J. Immunol. Meth. 266, 33-44.
Li, Z. Z., Gong, F. C., Shen, G. L., Yu, R. Q. (2002) Bacteria-modified amperometric immunosensor for a Brucella melitensis antibody assay. Anal. Sci. 18, 625-30.
Liu, G. D., Wu, Z. Y., Wang, S. P., Shen, G. L., Yu, R. Q. (2001) Renewable amperometric immunosensor for Schistosoma japonium antibody assay. Anal. Chem. 73, 3219-26.
Metz, C. E., Herman, B. A., Roe, C. A. (1998) Statistical comparison of two ROC curve estimates obtained from partially-paired datasets. Med. Decis. Making 18, 110-121
Van Pelt, W., van de Giessen, A., van Leeuwen, W., Wannet, W., Henken, A., Evers, E., de Wit, M., van Duynhoven, Y. (1999) Oorsprong, omvang en kosten van humane salmonellose. Deel 1. Oorsprong van humane salmonellose met betrekking tot varken, rund, kip, ei en overige bronnen. Infectieziekten Bull. 10, 240-243. In Dutch.
Proux, K., Jouy, E., Houdayer, C., Protais, J., Dibb-Fuller, M., Boscher, E., Gillard, A., Gracieux, P., Gilbert, F., Beaumont, C., Duchet-Suchaux, M. (2002) Reliable ELISAs showing differences between resistant and susceptible lines in hens orally inoculated with Salmonella enteritidis. Vet. Res. 33, 23-33.
Pyrohova, L. V., Starodub, M. F., Artiukh, V. P., Nahaieva, L. I., Dobrosol, H. I. (2002) Express diagnostics of bovine leucosis by immune sensor based on surface plasmon resonance. Ukr. Biokhim. Zh. 74, 88-92. In Ukrainian.
Sachsenweger, O., Lohr, J. E., Kosters, J. (1994) Evaluation of three commercial ELISA test kits for the detection of antibodies against Salmonella enteritidis. Tierarztl. Prax. 22, 350-7. In German.
Skov, M. N., Feld, N. C., Carstensen, B., Madsen, M. (2002) The serologic response to Salmonella enteritidis and Salmonella typhimurium in experimentally infected chickens, followed by an indirect lipopolysaccharide enzyme-linked immunosorbent assay and bacteriologic examinations through a one-year period. Avian Dis. 46, 265-73.
Su, X., Low, S., Kwang, J., Chew, V. H. T., Li, S. F. Y. (2001). Piezoelectric quartz crystal based veterinary diagnosis for Salmonella enteritidis infection in chicken and egg. Sensors and Actuators B: Chemical 75, 29-35.
Sunwoo, H. H., Nakano, T., Dixon, W. T., Sim, J. S. (1996) Immune responses in chickens against lipopolysaccharide of Escherichia coli and Salmonella typhimurium. Poult. Sci. 75, 342-5.
Uttenthaler, E., Kosslinger, C. and Drost, S. (1998) Characterization of immobilization methods for African swine fever virus protein and antibodies with a piezoelectric immunosensor. Biosens. Bioelectron. 13, 1279-86.
Vetcha, S., Wilkins, E., Yates, T. (2002) Detection of hantavirus infection in hemolyzed mouse blood using alkaline phosphatase conjugate. Biosens. Bioelectron. 17, 901-9.
De Vries, N., Zwaagstra, K. A., Huis in't Veld, J. H., van Knapen, F., van Zijderveld, F. G. and Kusters J. G. (1998) Production of monoclonal antibodies specific for the i and 1,2 flagellar antigens of Salmonella typhimurium and characterization of their respective epitopes. Appl. Environ. Microbiol. 64, 5033.
Van Zijderveld, F. G., van Zijderveld-van Bemmel, A. M. and Anakotta, J. (1992) Comparison of four different enzyme-linked immunosorbent assays for serological diagnosis of Salmonella enteritidis infections in experimentally infected chickens. J. Clin. Microbiol. 30, 2560-6.
Zweig, N. H. and Campbell, G. (1993) Receiver-operating characteristic (ROC) plots: a fundamental evaluation tool in clinical medicine. Clin. Chem. 39, 561-77. Erratum in: Clin Chem 1993, 39, 1589.

### Reference Example 3

Benoit P. W. and D. W. Donahue. 2003. Methods for rapid separation an concentration of bacteria in food that bypass time-consuming cultural enrichment. J. Food Prot. 66(10):1935-1948.
Che Y., Y. Li, and M. Slavik. 2001. Detection of Campylobacter jejuni in poultry samples using an enzyme-linked immunoassay coupled with an enzyme electrode. Biosens. Bioelectron 16(9-12):791-797.
Christensen B., H. Sommer, H. Rosenquist and N. Neilsen. 2001. Risk assessment on Campylobacter jejuni in chicken product. Available Source: http://www.foodrisk.org/risk_assessments.cfm?item1=Campylobacter&item2=All+Commodities&Submit=Submit, Jun. 15, 2004.
Coker A. O. 2000. Incidence, trends and sources of Campylobacteriosis in developing countries—An overview, pp. 44-48. In Proceedings of a WHO Consultation of Experts, Copenhagen, Denmark, 21-25 Nov. 2000. Available Source: http://www.who.int/emcdocuments/zoonoses/whocdscsraph20017c.html, Jun. 15, 2004.
FSAI (Food Safety Authority of Ireland). 2002. Control of Campylobacter species in the food chain. Available source: http://www.fsailefoublications/report/Campylobacter_report.pdf, Jun. 15, 2004.
Lake, R., A. Hudson, P. Cressey and G. Nortje. 2003. Risk Profile: Campylobacter jejuni/coli in poultry (whole and pieces). Client. Report FW0109. Christchurch: ESR. (Institute of Environmental Science & Research Limited). Available source: http://www.nzfsa.govt.nz/science-technology/riskprofiles/campylobacter.pdf, Jun. 15, 2004.
Stern, N. J. and J. E. Line. 2000. Campylobacter, pp. 1040-1056. In Lund B. M., T. C. Baird-Parker and G. W. Gould (eds.). The Microbiological Safety and Quality of Food Volume II. Aspen Publishers Inc., Gaithersburg, Md.
Tauxe R. V. 2000. Major risk factors for human Campylobacteriosis—An overview, pp. 65-66. In Proceedings of a WHO Consultation of Experts, Copenhagen, Denmark, 21-25 Nov. 2000. Available Source: http://www.whoint/emc-documents/zoonoses/whocdscsraph20017c.html, Jun. 15, 2004.
Waller D. F. and S. A. Ogata. 2000. Quantitative immunocapture PCR assay for detection of Campylobacter jejuni in foods. Appl. Environ. Microbiol. 66(9): 4115-4118.
Yu L. S., J. Uknalis, and S. I. Tu. 2001. Immunomagnetic separation methods for the isolation of Campylobacter jejuni from ground poultry meats. J. Immunol. Methods. 256(1-2): 11-18.

### References Example 4

Salinpork: Final Report of Salmonella in Pork (SALINPORK) Preharvest and harvest control options based on epidemiology, diagnostic and economic research (2000) EU project FAIR1 CT95-0400, (Lo Fo Wong, D. M. A. & Hald, T, eds), Copenhagen, Denmark, pp. 251.
European Commission—Health & Consumer Protection. Directorate General (2004) Trends and sources of zoonotic agents in animals, feedingstuffs, food and man in the European Union and Norwayin 2002, Part 1, SANCO/29/2004, pp. 32

### References Examples 6, 7 and 8

Lindberg, A A. Bacterial surface carbohydrates and bacteriophage adsorption. In: Sutherland I E., editor; Sutherland I E., editor. Surface carbohydrates of the prokaryotic cells. London, United Kingdom: Academic Press; 1977. pp. 289-356.
Lindberg A A, Holme T. 1969. Influence of O side chains on the attachment of the Felix O-1 bacteriophage to Salmonella bacteria. J. Bacteriol. 99(2):513-9.
Hirsh, Dwight C. and Martin, Lori D. 1983. Detection of Salmonella spp. in milk by using Felix-O1 bacteriophage and high-pressure liquid chromatography. Appl Environ. Microbiol. 46(5):1243-5.

1.-36. (canceled)

37. A method for immobilizing a polysaccharide on a carrier, the method comprising:
contacting the polysaccharide with an oxidizing agent and a polymer comprising at least two amine groups and/or amide groups to obtain a polysaccharide-polymer complex, and
coupling the polysaccharide-polymer complex to the carrier.

38. The method according to claim 37, wherein the polysaccharide is derived from a gram-negative bacterium, an enterobacteriaceae, a salmonella species or subspecies, or a lipopolysaccharide.

39. The method according to claim 37, wherein the polymer is protein.

40. The method according to claim 39, wherein the protein is hemoglobin or myoglobin.

41. The method according to claim 37, wherein the oxidizing agent is m-periodate or sodium m-periodate.

42. The method according to claim 37, further comprising:
activating the surface of the carrier.

43. The method according to claim 37, wherein the carrier comprises a glass surface coated with gold.

44. The method according to claim 37, wherein the carrier is modified with a coating comprising a carboxyl group donor, a carboxymethylated dextran layer, a carboxymethylated dextran layer activated with 1-ethyl-3-(3-dimethylaminopropyl)carbodiimide hydrochloride, N-hydroxysuccinimide, or carbohydrazide.

45. The method according to claim 37, wherein the carrier is a biosensor chip.

46. A carrier having a surface, the carrier comprising:
an immobilized polysaccharide-protein complex on the surface.

47. The carrier of claim 46 obtained by a method comprising:
contacting the polysaccharide with an oxidizing agent and a polymer comprising at least two amine and/or amide groups to obtain a polysaccharide-polymer complex, and
coupling the polysaccharide-polymer complex to the carrier.

48. The carrier of claim 47, wherein the carrier comprises a coating comprising a carboxyl group donor, a carboxymethylated dextran, linked to a polysaccharide comprising an antigen, wherein the carboxyl group donor and the polysaccharide are linked to each other via a polymer comprising at least two amine and/or amide groups, wherein at least the polysaccharide is linked to the polymer via a periodate oxidized vincinal diol on the polysaccharide and an amine and/or amide group on the polymer.

49. The carrier of claim 48, which is a microsphere a bead, a polystyrene microsphere, or polystyrene bead.

50. The carrier of claim 47, which is coded.

51. The carrier of claim 50, wherein the carrier is coded with a label comprising a color, fluorescent color, or phosphorescent color.

52. A collection of microspheres or beads comprising at least two differently coded carriers of claim 50.

53. The collection of microspheres or beads of claim 52, wherein each of the differently encoded microsphere or beads comprises a polysaccharide comprising a different antigen.

54. The carrier of claim 53 wherein the biosensor is a Surface Plasmon Resonance detection system.

55. The carrier of claim 47 incorporated in a biosensor.

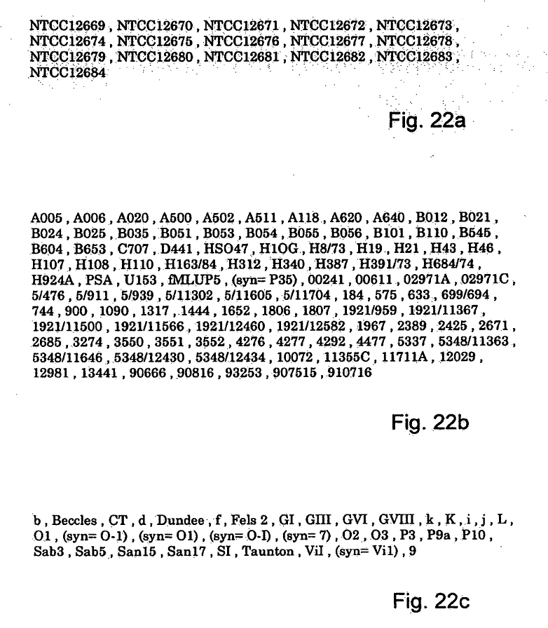

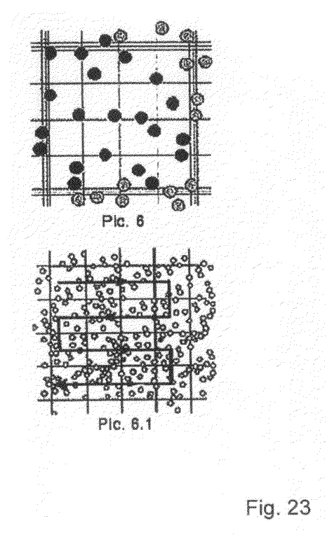

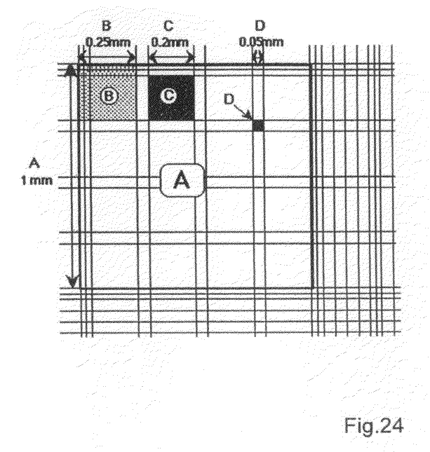

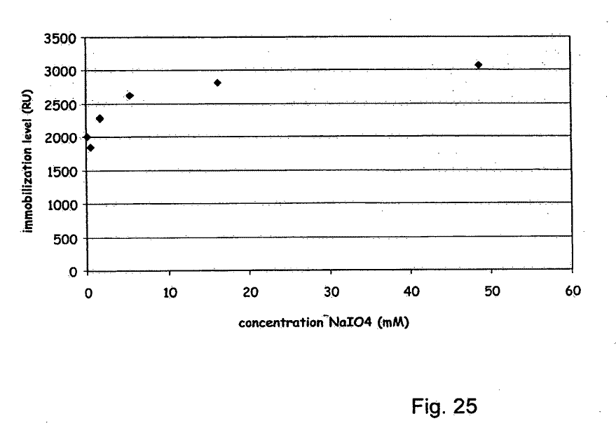

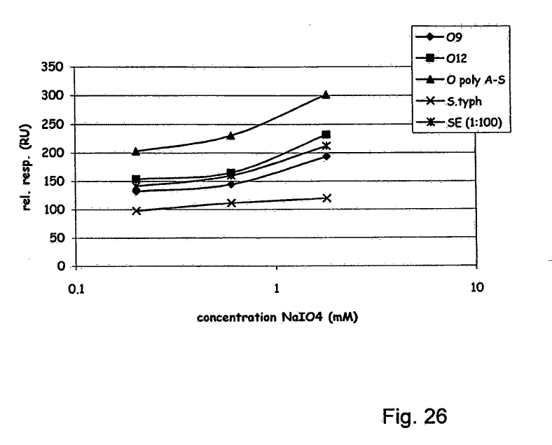

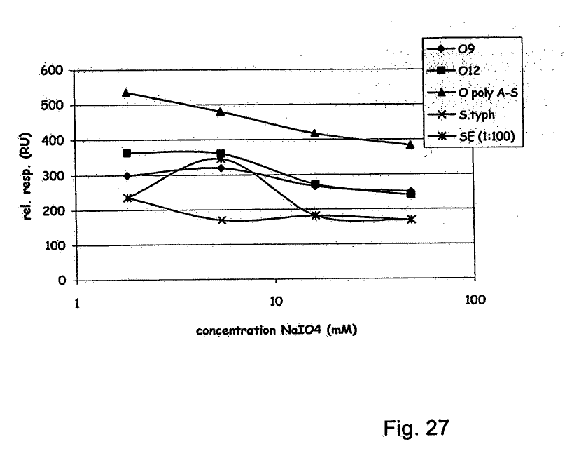

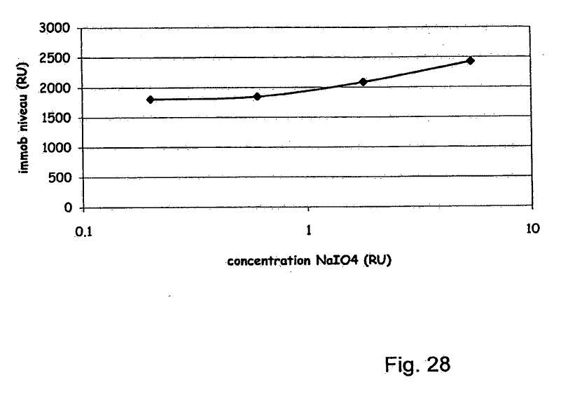

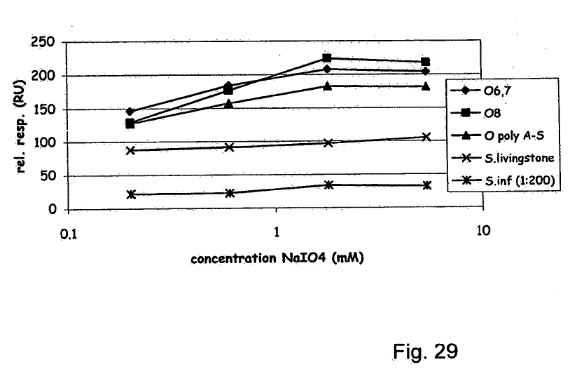

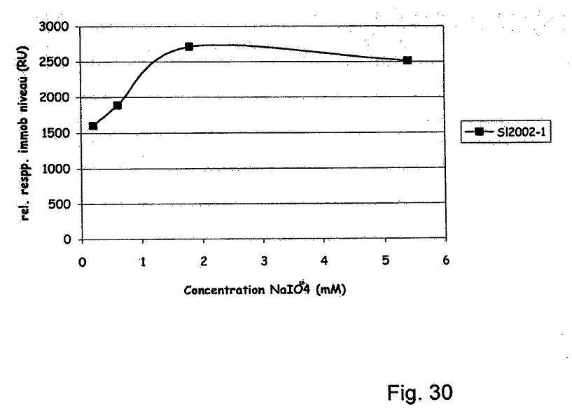

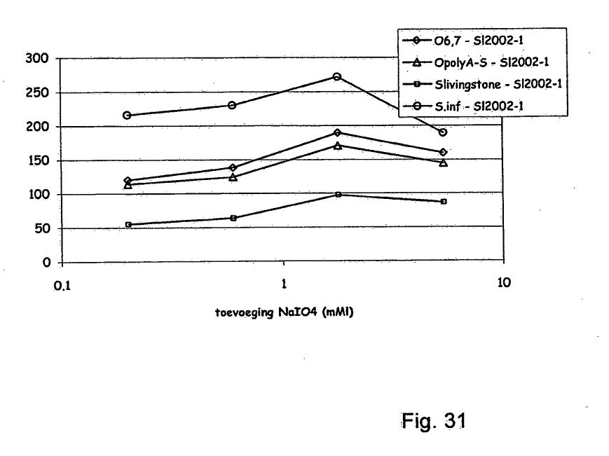

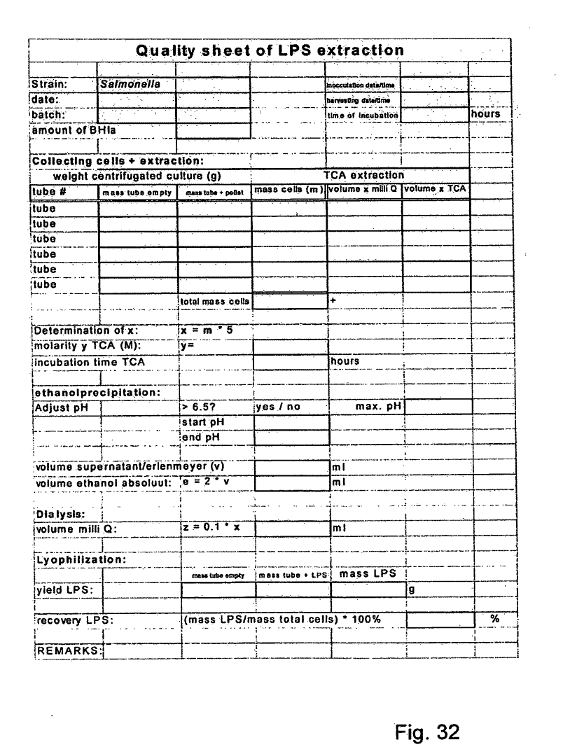

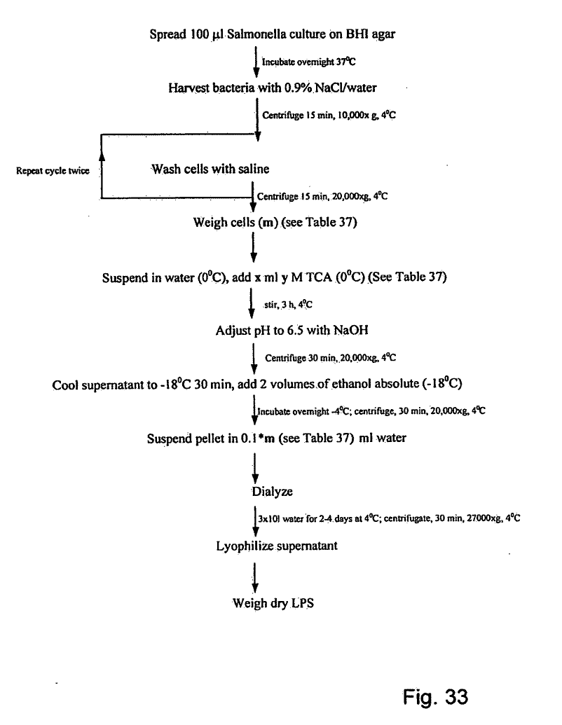

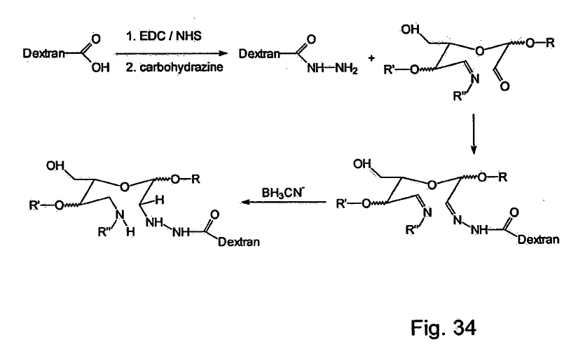

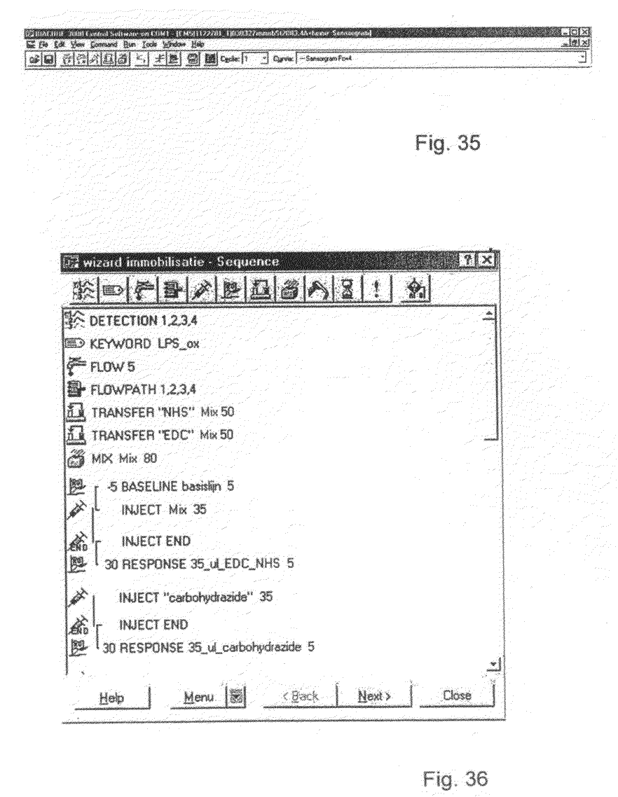

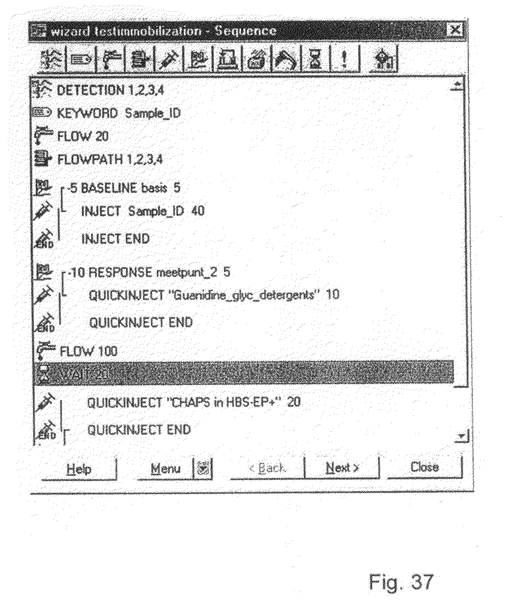

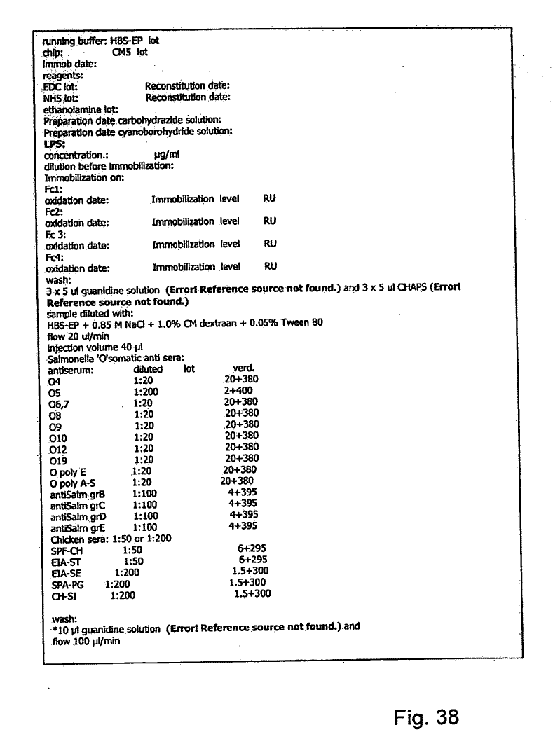

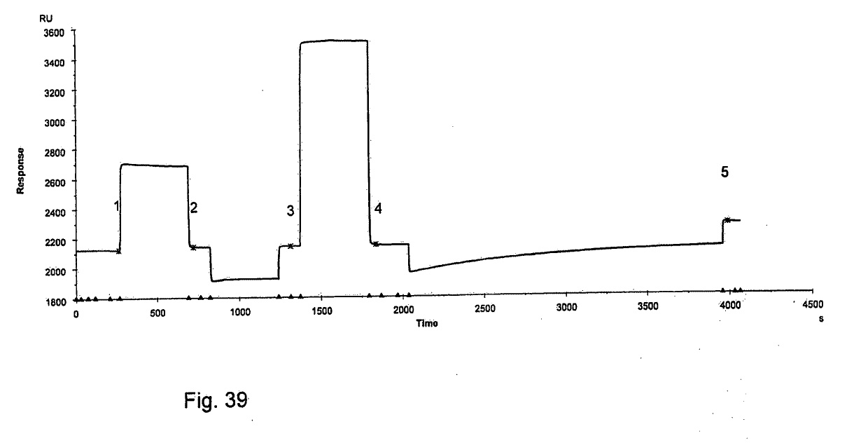

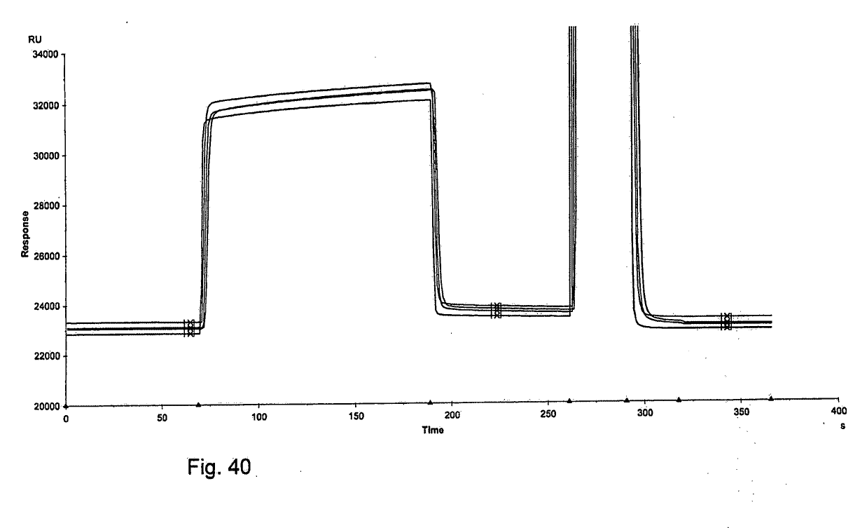

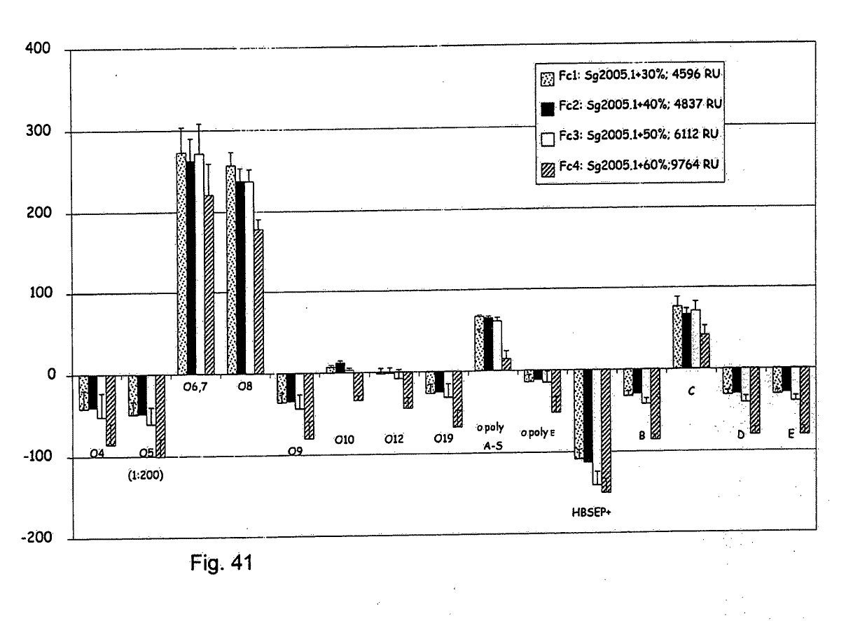

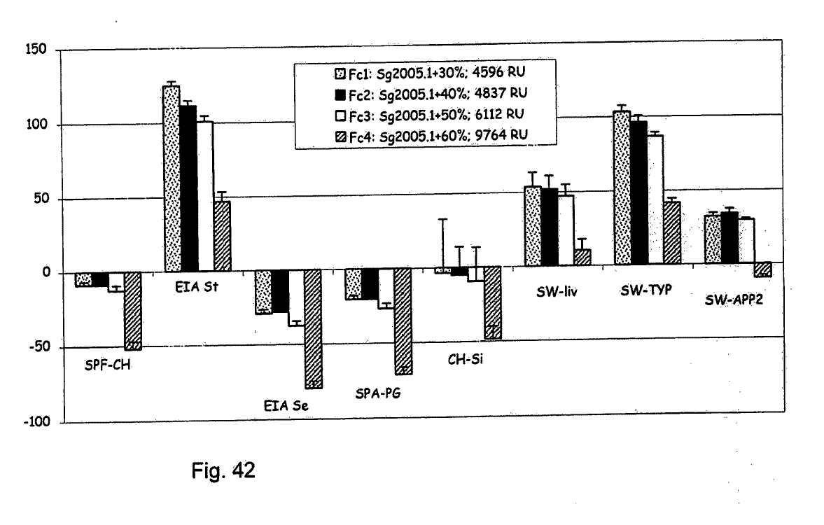

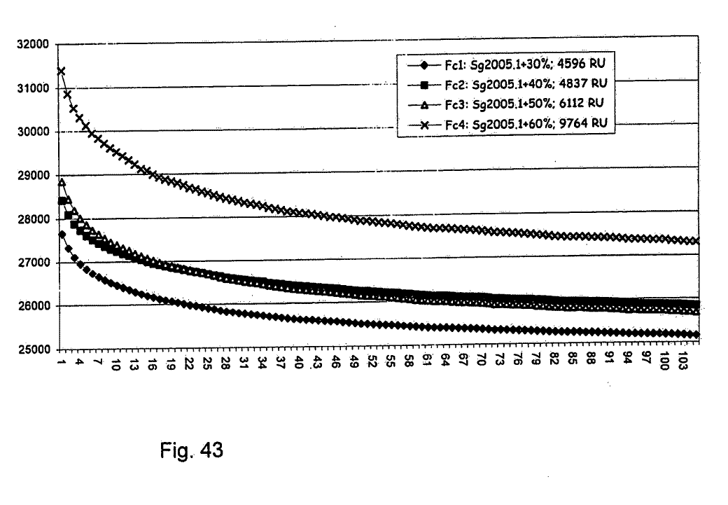

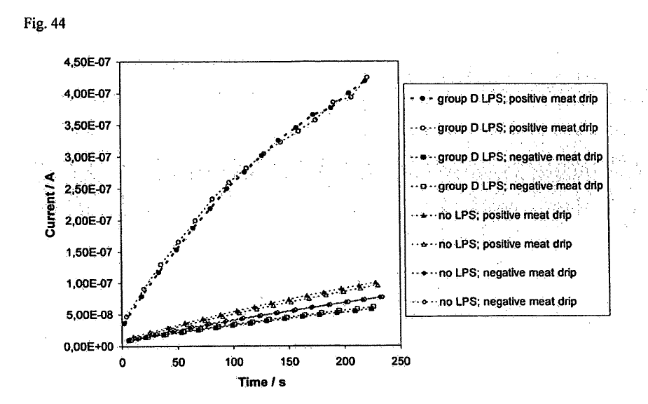

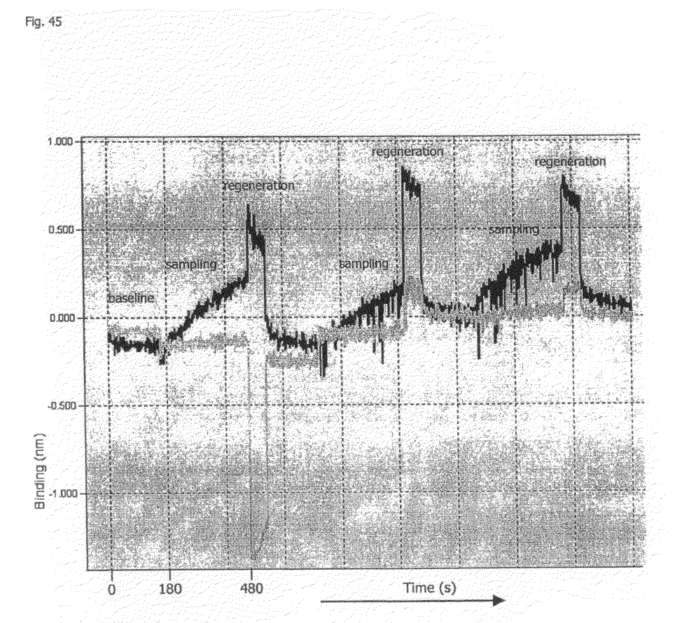

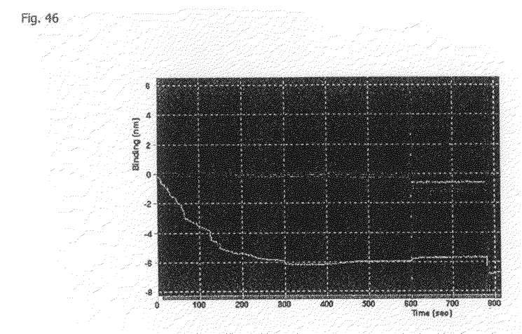

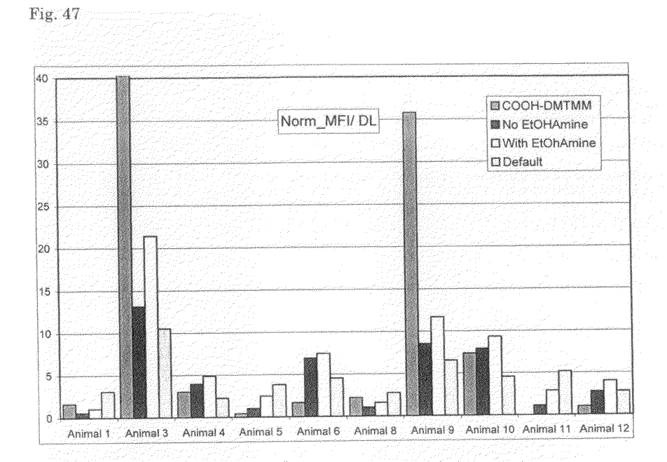

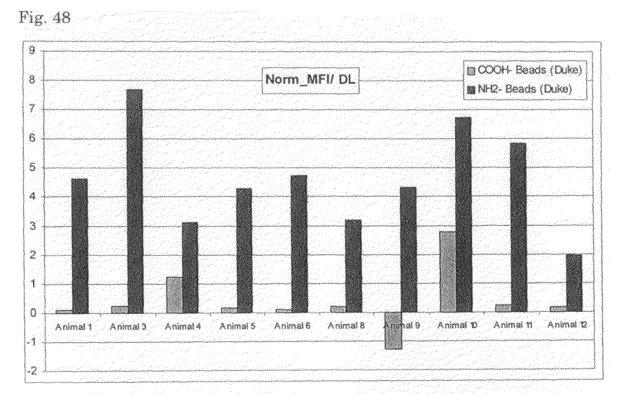

56. The carrier of claim 47 comprising a bacteriophage of FIG. 22a, 22b, and/or 22c.

57. A method for determining the presence of an antibody directed to an antigen of a gram-negative bacteria in a sample, the method comprising:
contacting the sample with the carrier of claim 47, and
determining whether the carrier has bound any antibody.

58. The method according to claim 57, wherein the sample is blood, blood-derived liquid material, tissue-derived fluids, meat drip, milk, egg, fluids from an eye, fluids from saliva, or fluids from feces.

59. The method according to claim 57, wherein binding to the carrier is determined by Surface Plasmon Resonance.

60. A method for determining the presence of a gram-negative bacterium in a sample, the method comprising:
(a) (i) contacting the sample with a predetermined amount of antibodies directed against an antigen of the gram-negative bacterium and
(a) (ii) determining the amount of antibodies not bound to the bacterium with the carrier of claim 47, or
(b) (i) contacting the sample with target bacteria-specific bacteriophages;
(b) (ii) allowing the bacteriophages to infect the sample;
(b) (iii) removing non-bound and/or non-invading bacteriophages resulting in a bacteriophage infected sample;
(b) (iv) bringing the bacteriophage infected sample into contact with an indicator organism susceptible for the used bacteriophages;
(b) (v) incubating during at least one bacteriophage multiplication cycle;
(b) (vi) recovering the bacteriophages to obtain a bacteriophage-containing sample, and
(b) (vii) analyzing the bacteriophage-containing sample with the carrier.

61. The method according to claim 60, wherein binding to the carrier is determined by Surface Plasmon Resonance.

62. The method according to claim 60, wherein the sample is obtained from a human, a plant, or an animal.

63. The method according to claim 60, wherein the bacteriophage comprises a bacteriophage of FIG. 22a, 22b and/or 22c.
